SEQUENCE LISTING (34 sequences)

SEQ ID NO: 1 - protein, 9 aa, synthetic construct
  REGION 1..9: note = synthetic polypeptide
  VARIANT 4: note = UV-cleavable amino acid residue
FMYXDFHFI

SEQ ID NO: 2 - protein, 10 aa, synthetic construct
  REGION 1..10: note = synthetic polypeptide
  VARIANT 4: note = UV-cleavable amino acid residue
FLPXDFFPSV

SEQ ID NO: 3 - protein, 10 aa, synthetic construct
  REGION 1..10: note = synthetic polypeptide
  VARIANT 6: note = UV-cleavable amino acid residue
FLPSDXFPSV

SEQ ID NO: 4 - protein, 9 aa, synthetic construct
  REGION 1..9: note = synthetic polypeptide
  VARIANT 6: note = UV-cleavable amino acid residue
FYIQMXTEL

SEQ ID NO: 5 - protein, 9 aa, synthetic construct
  REGION 1..9: note = synthetic polypeptide
  VARIANT 4: note = UV-cleavable amino acid residue
YVIXDLAAM

SEQ ID NO: 6 - protein, 9 aa, synthetic construct
  REGION 1..9: note = synthetic polypeptide
  VARIANT 4: note = UV-cleavable amino acid residue
HFFXWGTMF

SEQ ID NO: 7 - protein, 10 aa, synthetic construct
  REGION 1..10: note = synthetic polypeptide
  VARIANT 6: note = UV-cleavable amino acid residue
AVVSLXRLLK

SEQ ID NO: 8 - protein, 9 aa, synthetic construct
  REGION 1..9: note = synthetic polypeptide
  VARIANT 4: note = UV-cleavable amino acid residue
GTHXLLPFY

SEQ ID NO: 9 - protein, 9 aa, synthetic construct
  REGION 1..9: note = synthetic polypeptide
  VARIANT 6: note = UV-cleavable amino acid residue
AMLTAXFLR

SEQ ID NO: 10 - protein, 9 aa, synthetic construct
  REGION 1..9: note = synthetic polypeptide
  VARIANT 6: note = UV-cleavable amino acid residue
HLMFYXLPI

SEQ ID NO: 11 - protein, 9 aa, synthetic construct
  REGION 1..9: note = synthetic polypeptide
  VARIANT 4: note = UV-cleavable amino acid residue
QLFXFSPRR

SEQ ID NO: 12 - protein, 10 aa, synthetic construct
  REGION 1..10: note = synthetic polypeptide
  VARIANT 2: note = UV-cleavable amino acid residue
TXFFYRYGFV

SEQ ID NO: 13 - protein, 9 aa, synthetic construct
  REGION 1..9: note = synthetic polypeptide
  VARIANT 4: note = UV-cleavable amino acid residue
DEFXPIVQY

SEQ ID NO: 14 - protein, 9 aa, synthetic construct
  REGION 1..9: note = synthetic polypeptide
  VARIANT 6: note = UV-cleavable amino acid residue
RESFGXESF

SEQ ID NO: 15 - protein, 9 aa, synthetic construct
  REGION 1..9: note = synthetic polypeptide
  VARIANT 4: note = UV-cleavable amino acid residue
TPAXYFHVL

SEQ ID NO: 16 - protein, 9 aa, synthetic construct
  REGION 1..9: note = synthetic polypeptide
  VARIANT 4: note = UV-cleavable amino acid residue
AENXYVTVF

SEQ ID NO: 17 - protein, 9 aa, synthetic construct
  REGION 1..9: note = synthetic polypeptide
  VARIANT 8: note = UV-cleavable amino acid residue
KEVLVLWXI

SEQ ID NO: 18 - protein, 9 aa, synthetic construct
  REGION 1..9: note = synthetic polypeptide
  VARIANT 6: note = UV-cleavable amino acid residue
FMYEGXTPL

SEQ ID NO: 19 - protein, 9 aa, synthetic construct
  REGION 1..9: note = synthetic polypeptide
  VARIANT 4: note = UV-cleavable amino acid residue
FPFXLAAII

SEQ ID NO: 20 - protein, 9 aa, synthetic construct
  REGION 1..9: note = synthetic polypeptide
  VARIANT 6: note = UV-cleavable amino acid residue
FPIPSXWAF

SEQ ID NO: 21 - protein, 9 aa, synthetic construct
  REGION 1..9: note = synthetic polypeptide
  VARIANT 8: note = UV-cleavable amino acid residue
ITAAAWYXW

SEQ ID NO: 22 - protein, 9 aa, synthetic construct
  REGION 1..9: note = synthetic polypeptide
  VARIANT 6: note = UV-cleavable amino acid residue
LAVMGXAAW

SEQ ID NO: 23 - protein, 9 aa, synthetic construct
  REGION 1..9: note = synthetic polypeptide
  VARIANT 4: note = UV-cleavable amino acid residue
HLPXGVKSL

SEQ ID NO: 24 - protein, 8 aa, synthetic construct
  REGION 1..8: note = synthetic polypeptide
  VARIANT 6: note = UV-cleavable amino acid residue
FAAEAXKL

SEQ ID NO: 25 - protein, 9 aa, synthetic construct
  REGION 1..9: note = synthetic polypeptide
  VARIANT 6: note = UV-cleavable amino acid residue
GAINSXLPY

SEQ ID NO: 26 - protein, 9 aa, synthetic construct
  REGION 1..9: note = synthetic polypeptide
  VARIANT 6: note = UV-cleavable amino acid residue
FAIVPXLQI

SEQ ID NO: 27 - protein, 9 aa, synthetic construct
  REGION 1..9: note = synthetic polypeptide
  VARIANT 4: note = UV-cleavable amino acid residue
FAMXVPLLI

SEQ ID NO: 28 - protein, 9 aa, synthetic construct
  REGION 1..9: note = synthetic polypeptide
  VARIANT 4: note = UV-cleavable amino acid residue
ARFXDLRFV

SEQ ID NO: 29 - protein, 9 aa, synthetic construct
  REGION 1..9: note = synthetic polypeptide
  VARIANT 4: note = UV-cleavable amino acid residue
ANNXRLWVY

SEQ ID NO: 30 - protein, 9 aa, synthetic construct
  REGION 1..9: note = synthetic polypeptide
  VARIANT 4: note = UV-cleavable amino acid residue
YAAXTNFLL

SEQ ID NO: 31 - protein, 9 aa, synthetic construct
  REGION 1..9: note = synthetic polypeptide
  VARIANT 6: note = UV-cleavable amino acid residue
ISDSAXNMM

SEQ ID NO: 32 - protein, 9 aa, synthetic construct
  REGION 1..9: note = synthetic polypeptide
  VARIANT 4: note = UV-cleavable amino acid residue
WAWXFAAVL

SEQ ID NO: 33 - protein, 9 aa, synthetic construct
  REGION 1..9: note = synthetic polypeptide
  VARIANT 4: note = UV-cleavable amino acid residue
MMHXSTSPF

SEQ ID NO: 34 - protein, 9 aa, synthetic construct
  REGION 1..9: note = synthetic polypeptide
  VARIANT 6: note = UV-cleavable amino acid residue
RTFGQXLFF

(19) United States
(12) Patent Application Publication
(10) Pub. No.: US 2023/0349903 A1
(43) Pub. Date: Nov. 2, 2023

(54) ASSAYS AND REAGENTS FOR CHARACTERIZATION OF MHCI PEPTIDE BINDING

(71) Applicant: GENENTECH, INC., South San Francisco, CA (US)

(72) Inventors: Juan LI, South San Francisco, CA (US); Wilson PHUNG, South San Francisco, CA (US); Wendy Noel SANDOVAL, South San Francisco, CA (US); Whitney Lauren BINDER, South San Francisco, CA (US); Craig BLANCHETTE, South San Francisco, CA (US); Pamela Pui Fung CHAN, South San Francisco, CA (US); Martine Abraham DARWISH, South San Francisco, CA (US)

(21) Appl. No.: 18/173,002

(22) Filed: Feb. 22, 2023

Related U.S. Application Data
(63) Continuation of application No. PCT/US2021/047537, filed on Aug. 25, 2021.
(60) Provisional application No. 63/218,073, filed on Jul. 2, 2021.
(60) Provisional application No. 63/085,113, filed on Sep. 29, 2020.
(60) Provisional application No. 63/070,211, filed on Aug. 25, 2020.

(51) Int. Cl. G01N 33/569; C07K 14/74; G01N 33/68

(52) U.S. Cl. CPC G01N 33/56977; C07K 14/70539; G01N 33/6848; G01N 2333/70539; G01N 2800/24

(57) ABSTRACT

The present disclosure relates to reagents and methods of making and for detecting MHCI/ligand peptide complexes.

### CROSS-REFERENCES TO RELATED APPLICATIONS

This application is a bypass continuation application claiming priority to International Application No. PCT/US2021/047537, filed on Aug. 25, 2021, which claims priority to U.S. Provisional Application Nos. 63/070,211, filed Aug. 25, 2020; 63/085,113, filed Sep. 29, 2020; and 63/218,073, filed Jul. 2, 2021, each of which are incorporated herein by reference in their entirety for all purposes.

### SEQUENCE LISTING

The instant application contains a Sequence Listing which has been submitted electronically in ASCII format and is hereby incorporated by reference in its entirety. Said ASCII copy, created on Feb. 22, 2023, is named 048893-533C01US_ST26.xml and is 56,318 bytes in size.

### FIELD

The present application relates to systems and methods for analyzing major histocompatibility class I (MHCI) complexes. Analysis may be performed using native mass spectrometry, enzyme-linked immunosorbent assay (ELISA), time-resolved fluorescence resonance energy transfer (TR-FRET) spectroscopy, optionally in combination with MHCI complex isolation via size exclusion chromatography or capillary electrophoresis, such as capillary zone electrophoresis. In some embodiments, the methods are applied to peptide-exchanged MHCI complexes, for example, using peptides predicted to be present in patient samples.

### BACKGROUND

The major histocompatibility complex-I (MHCI) is an almost ubiquitously expressed protein complex that is responsible for presenting self- and foreign-derived display peptides on the surfaces of antigen presenting cells to lymphocytes. Display peptide presentation by MHCI is one of the first steps of an adaptive immune response toward destruction of diseased cells or for preservation of healthy cells. The MHCI complex is a non-covalently linked protein heterodimer consisting of a heavy chain (α) and light chain (β₂ microglobulin, B2M); in general, the MHCI complex is unstable without an 8-11 residue display peptide ligand. The conical display peptide generation pathway enlists the proteasome, which degrades ubiquitinated cytosolic proteins into potential display peptides. These display peptides are subsequently imported into the endoplasmic reticulum where they are further refined, and the active MHCI/display peptide complex is formed via a protein chaperone-assisted process before transport to the cell surface for presentation to cytotoxic, or CD8(+) T-cells, to recognize and determine the cellular fate.

The MHCI proteins are encoded by the major histocompatibility complex gene complex, and are also known as members of the human leukocyte antigen (HLA) system. The most common MHCI family members are encoded by the HLA-A, HLA-B, and HLA-C loci, although there are approximately 24 total known for the MHCI family. Each HLA group contains at least a dozen or more alleles, and differential expression of these alleles leads to a rich diversity of protein outputs. Indeed, there are >20,000 possible different HLA-A, HLA-B, and HLA-C proteins complexes, each with their own stability and canonical ligand specificity. The high diversity of the MHCI system of proteins enable the system as a whole to recognize a large number of possible antigens, including peptides derived from non-human sources, post-translationally modified self-peptides, and peptides synthesized ex-vivo.

An evolving area of interest for treating elusive immune targets involves generation of previously uncharacterized, unknown, or designed antigens, including neoantigen peptide sequences, for characterizing CD8(+) T-cell-dependent responses in patients. However, there is a lack of robust, high-throughput methods available for production, selection, and identification of optimal MHCI complex:antigen binding in vitro, including for neoantigens.

### SUMMARY

The instant application provides compositions and methods for assaying MHCI peptides for use in a wide range of immunotherapies.

Described herein is a rapid, high-throughput, multiplexed monitoring assay to look for potential neoantigen peptides that are likely to be presented on a patient's MHCI molecules, and that may bind to T-cells may be useful in developing appropriate treatments for cancer patients. Assays such as ELISA (enzyme-linked immunosorbent assay), TR-FRET (time-resolved fluorescence resonance energy transfer), and 2D-LC-MS (two-dimensional liquid chromatography mass spectrometry) may be used for analyzing MHCI-peptide complexes. Chromatography such as 2D-LC may be combined with mass spectrometry (MS) for detection.

Methods and systems described herein utilize native mass spectrometry to characterize MHCI complexes bound to peptides. In some embodiments, native mass spectrometry is preceded by size exclusion chromatography (SEC) or in other cases by capillary electrophoresis (CE), such as capillary zone electrophoresis (CZE). In some cases, native mass spectrometry allows for the characterization of peptides bound to an MHCI complex and confirmation that particular peptides are non-covalently bound to an MHCI complex. CE, in some cases, may also allow for detection of bound peptides present at lower concentrations than chromatography methods such as SEC. In some cases, only a single chromatography separation by SEC, CE or CZE is performed followed by the native mass spectrometry, as opposed to a two-dimensional separation in 2D-LC-MS methods for example. In some embodiments, methods herein may also provide increased throughput compared to other analytical techniques.

In an aspect, provided herein, is a major histocompatibility complex class I (MHCI) protein complex including an alpha chain, a beta chain, and a ligand, the ligand comprising a non-natural UV-cleavable amino acid.

In an aspect, provided herein, is a peptide exchange assay for determining binding of a MHCI allele to a test peptide by providing a first composition comprising a test peptide and a MHCI/ligand complex including (i) a MHCI molecule comprising an alpha chain, a beta chain, and (ii) a ligand, wherein the ligand is a peptide comprising a non-natural ultraviolet (UV)-cleavable amino acid; exposing the first composition to UV light to cleave the ligand at the UV-cleavable amino acid; and incubating the first composition for a period of time to form a second composition comprising free test peptide, the alpha chain, the beta chain, and/or a MHCI/-second peptide complex; and determining whether the MHCI allele is bound to the second peptide.

In an aspect, provided herein, is a method for determining optimal MHCI allele-ligand combinations, the method involving: providing a plurality of MHCI alpha chain monomers that were purified under denaturing conditions, forming a reaction mixture by combining the plurality of MHCI alpha chain monomers, a plurality of beta chain monomers, and a ligand containing a non-natural UV-cleavable amino acid, incubating the reaction mixture under conditions to allow formation of a MHCI-ligand complex, and determining whether the MHCI-ligand complex was formed.

In an aspect, provided herein, is a method for detecting the binding of a MHCI allele to a test peptide, the method involving: providing a first complex including a test peptide and a MHCI/ligand complex, including a MHCI molecule that includes an alpha chain, a beta chain, and a ligand, where the ligand is a peptide that includes an non-natural, ultraviolet (UV)-cleavable amino acid, then exposing the first complex to UV light to cleave the ligand at the UV-cleavable amino acid, and detecting a MHCI/test peptide complex in the second complex, thereby detecting the binding of the MHCI molecule to the test peptide.

In an aspect, provided herein, is a method of identifying a MHCI binding ligand, the method including contacting a plurality of MHCI allele chain monomers with a plurality of beta chain monomers and a ligand under conditions that allow for the formation of a MHCI/ligand complex, wherein the ligand is a peptide containing a non-natural UV-cleavable amino acid, and detecting the MHCI/ligand complex, thereby identifying a MHCI binding ligand.

In an aspect, provided herein, is a method for determining optimal major histocompatibility complex class I (MHCI) allele-ligand combinations, the method including providing a plurality of MHCI alpha chain monomers purified under denaturing conditions; forming a reaction mixture by combining the plurality of MHCI alpha chain monomers, a plurality of beta chain monomers, and a ligand comprising a peptide comprising a non-natural UV-cleavable amino acid; incubating the mixture under conditions to allow formation of a MHCI/ligand complex; and determining whether the MHCI/ligand complex was formed.

In an aspect, provided herein, is a kit containing a peptide comprising a non-natural UV-cleavable amino acid, MHCI alpha chain monomers, and MHCI beta chain monomers.

In an aspect, provided herein, is a system containing: a peptide containing a non-natural UV-cleavable amino acid; a plurality of MHCI alpha chain monomers; a plurality of MHCI beta chain monomers; and a first reagent capable of allowing formation of a MHCI/ligand complex.

The present disclosure further relates to methods for monitoring peptide-exchanged MHCI complexes using either size exclusion chromatography or capillary electrophoresis coupled with native mass spectroscopy.

In an aspect, provided herein, is a method of monitoring peptide-exchanged major histocompatibility class I (MHCI) complexes in a sample, including: (a) obtaining peptide-exchanged MHCI complexes comprising a peptide of interest; (b) performing size exclusion chromatography (SEC), capillary electrophoresis (CE), or capillary zone electrophoresis (CZE) on the peptide exchanged MHCI complexes; and (c) following the chromatography or capillary electrophoresis of (b), performing native mass spectrometry (MS) on the MHCI complexes to identify MHCI complexes that comprise peptides of interest.

In an aspect, provided herein, is a method of monitoring peptide-exchanged major histocompatibility class I (MHCI) complexes in a sample, including: (a) obtaining MHCI complexes comprising an exchangeable peptide and exposing the complexes to one or more peptides of interest under conditions which allow for peptide exchange between the exchangeable peptide and the peptide of interest; (b) performing size exclusion chromatography (SEC), capillary electrophoresis (CE), or capillary zone electrophoresis (CZE) on the peptide exchanged MHCI complexes; and (c) following the chromatography or capillary electrophoresis of (b), performing native mass spectrometry (MS) on the MHCI complexes to identify MHCI complexes that comprise peptides of interest.

In an aspect, provided herein, is a method of monitoring T-cell recognition of MHCI-complexed peptides, including: (a) obtaining peptide-exchanged MHCI complexes comprising a peptide of interest; (b) contacting the peptide-exchanged MHCI complexes with a sample comprising T-cells; (c) separating T-cell bound MHCI complexes from unbound MHCI complexes; (d) performing size exclusion chromatography (SEC), capillary electrophoresis (CE), or capillary zone electrophoresis (CZE) on the peptide exchanged MHCI complexes; and (e) following the chromatography or capillary electrophoresis of (d), performing native mass spectrometry (MS) on the MHCI complexes to identify MHCI complexes comprising peptides recognized by T-cells from the sample.

In an aspect, provided herein, is a method A method of monitoring T-cell recognition of MHCI-complexed peptides, including: (a) obtaining major histocompatibility class I (MHCI) complexes comprising an exchangeable peptide and exposing the complexes to one or more peptides of interest under conditions which allow for peptide exchange; (b) contacting the peptide-exchanged MHCI complexes with a sample comprising T-cells; (c) separating T-cell bound MHCI complexes from unbound MHCI complexes; (d) performing size exclusion chromatography (SEC), capillary electrophoresis (CE), or capillary zone electrophoresis (CZE) on the peptide exchanged MHCI complexes; and (e) following the chromatography or capillary electrophoresis of (d), performing native mass spectrometry (MS) on the MHCI complexes to identify MHCI complexes comprising peptides recognized by T-cells from the sample.

### BRIEF DESCRIPTION OF THE DRAWINGS

FIG. 1 is a cartoon depiction of a high-throughput assay to screen for peptide binders of MHCI. A pan-HLA capture antibody is attached to an ELISA plate. Unfolded HLA, denatured/renatured B2M, and peptides are mixed in a one-pot refolding reaction in the presence of the ELISA plate. Stable MHCI/B2M/peptide complexes are captured by the pan-HLA capture antibody. The relative number of MHCI/B2M/peptide complexes is determined by adding an anti-B2M biotinylated secondary antibody and subsequently adding a streptavidin-HRP complex for detection, via HRP-HRP substrate bioluminescence reaction.

FIGS. 2A-2B show bar graph representations of the normalized (vs. no peptide present, sample signal/negative control signal) ELISA signals of captured MHCI/B2M/peptide complexes (via secondary antibody reporter) averaged across binders. FIG. 2A shows the normalized signal within the range of 1-40 response units for 38 different HLA, HLB, and HLC alleles, and FIG. 2B shows the normalized signal within the range of 1-5 response units for the 38 HLA, HLB, and HLC alleles, refolded in the presence of B2M and peptide.

FIGS. 3A and 3B show bar graph representations of averaged ELISA signal over MHCI/B2M/peptide binders. The bar graph in FIG. 3A shows that some alleles have low affinity for the pan-HLA antibody used to label the ELISA plate. The bar graph in FIG. 3B shows some MHCI/B2M complexes are stable without the presence of peptide. The dashed line indicates the same signal level in A vs. B. Arrows indicate specific MHCI alleles that are low capture antibody binders (A), and specific MHCI/B2M complexes that are stable without the presence of peptide (B).

FIGS. 4A and 4B show bar graph representations of averaged ELISA signal over MHCI/B2M/UV peptide binders, in the presence of UV peptide (a peptide containing a UV-cleavable amino acid). FIG. 4A shows the overall normalized signal of MHCI/B2M/UV peptide complexes in the range of 1-40 response units. FIG. 4B shows the normalized signal at the range of 1-5 response units, indicating the specific alleles that form stable complexes without the presence of peptide (S) and alleles for which the capture antibody has low affinity (L).

FIG. 5 is a bar graph comparing normalized ELISA results and yield from a scaled up refolding purification of 18 different HLA/HLB alleles. The black bars indicate the relative signal generated by a formed MHCI/B2M/peptide complex as detected by ELISA. The grey bars indicate the % yield of producing 1 L of each MHCI/B2M/peptide complex. The horizontal line indicates a 1% cut-off for MHCI/B2M/peptide complex quality control. The designations (S), (L), refer to MHCI/B2M complexes stable without peptide, and MHC/B2M/peptide that the capture antibody has low affinity for, respectively.

FIG. 6 shows representative size-exclusion and reversed-phase chromatograms and mass spectra from a 2-D LC/MS characterization of a MHC/B2M/peptide exchange assay for three peptides.

FIG. 7 is a list of MHCI alleles and a peptide sequence that can be associated with the allele in embodiments herein. Each amino acid is represented by its standard single letter abbreviation. J represents a non-natural, UV cleavable amino acid.

FIGS. 8A-8C. FIG. 8A is a cartoon representation of a time-resolved fluorescence resonance transfer assay (TR-FRET) for detecting assembled MHCI/B2M/peptide complexes. FIG. 8B is a schematic representing a differential scanning fluorimetry (DSF) assay to measure thermal shift of melting temperature, thus binding, of MHCI complexes. FIG. 8C are representative spectra for HLA*03:01 peptide binders and non-binders, where the melting temperature (Tm) is shifted to indicate a change in enthalpy of complex formation.

FIGS. 9A and 9B show bar graph representations of TR-FRET assays to discover high-affinity peptides for different alleles. FIG. 9A plots the change in fluorescence at 665 nm at 37° C. and 4° C. for each complex tested. FIG. 9B is a bar graph of the relative accuracy of the TR-FRET assay vs. MHCI allele, varying from 85-100% accuracy.

FIGS. 10A-10D. FIG. 10A provides comparative DSF spectra comparing peptide binders and non-binders of MHCI HLA*03:01 containing complexes. At low temperatures (20° C.), relative fluorescence (RFU) is low for complexes where there is a peptide bound, and high for complexes without peptide binding. The peptide binder DSF spectra show a similar range of temperatures for Tm, whereas the non-binders have lower Tm. FIG. 10B is a bar graph of total number vs. RFU values for peptide binders (black) and non-binders (grey). FIG. 10C is a bar graph of total number vs. RFU values for peptide binders (black) and non-binders (grey). FIG. 10D is a bar graph of the % accuracy of the assay for four different MHCI alleles (all >90%).

FIGS. 11A-11D. FIG. 11A provides comparative DSF spectra comparing peptide binders and non-binders of MHCI HLA*08:01 containing complexes. At low temperatures (20° C.), relative fluorescence (RFU) is similar when there are peptide binders and non-binders. Both the peptide binder/non-binder DSF spectra show a similar range of temperatures for Tm. FIG. 11B is a bar graph of total number vs. Tm temperature for peptide binders (black) and non-binders (grey). FIG. 11C is a bar graph of total number vs. RFU values for peptide binders (black) and non-binders (grey). FIG. 11D is a bar graph of the % accuracy of the assay for four different MHCI alleles (60-85% accuracy)

FIG. 12 is a bar graph representation of TR-FRET percent “true binders” vs. predicted percentile rank. The percentile rank is a value calculated by running the peptide sequence through a prediction algorithm for peptide/MHCI binding (Nielsen M, et al. Protein Sci. (2003) 12:1007-1017, Andreatta M, and Nielsen, M. Bioinformatics (2016) Feb. 15; 32(4):511-517). Peptides with a percentile rank of less than 2.00 (left of the dashed line) are classified as MHCI binders by the algorithm. The methods and assays described herein identified a number of peptide-HLA combinations that were true binders but would have been classified as non-binders by the prediction algorithm.

FIG. 13 is a schematic for a 2-D LC/MS assay to determine MHCI/B2M/peptide exchange over time. MHCI/B2M/UV-cleavable peptide complexes are exposed to UV light, cleaving the peptide bond of the UV-cleavable peptide, in the presence of a second, exchange peptide. The cleaved fragment of the first peptide is exchanged for the full-length second peptide in the presence of the MHCI/B2M complex. At certain time points, the exchange mixture is analyzed by 1) “first dimension”: size-exclusion chromatography (SEC), which separates MHCI/B2M/peptide complexes from free MHCI, B2M, and peptide, 2) “second dimension”: reversed-phase HPLC of SEC peaks to separate peak components, and 3) mass spec analysis of the individual reversed-phase peaks for identification and quantification of peak components.

FIG. 14 shows a validation panel of 10 peptides as exchangers or non-exchangers for each allele, shown as a plot of % exchange over time for a range of peptides with the MHCI/B2M/UV-peptide complex as measured by 2-D LC/MS. The top data points indicate MHCI/peptide complexes where the peptide exchanged (observed in second HPLC peak) and no loss of MHCI peak (observed in first SEC peak) occurred. The middle data points indicate some loss of MHCI SEC peak area but retention of exchange peptide, and bottom data points indicate large loss of MHCI SEC peak and no exchange peptide.

FIG. 15 is a schematic for a 2-D LC/MS assay to determine MHCI/B2M/peptide exchange over time for a pool of 40 peptides. MHCI/B2M/UV-cleavable peptide complexes are exposed to UV light, cleaving the peptide bond of the UV-cleavable peptide, in the presence of a pool exchange peptides. The cleaved fragment of the first peptide is exchanged for the full-length peptides in the presence of the MHCI/B2M complex. At certain time points, the exchange mixture is analyzed by 1) size-exclusion chromatography (SEC), which separates MHCI/B2M/peptide complexes from free MHCI, B2M, and peptide, 2) reversed-phase HPLC of SEC peaks to separate peak components, and 3) mass spec analysis of the individual reversed-phase peaks for identification and quantification of peak components, including quantifying the number of different peptides from the exchange pool. The graph here shows the intensities of 10 known peptide binders in a pool of 40 peptides included in an exchange reaction with MHCI HLA-A*01:01 over time. This method allows for the identification of multiple peptide binders in a single run.

FIG. 16 shows a schematic diagram of an MHCI peptide exchange process using a UV-cleavable first peptide (light and dark colored circles), which, after UV cleavage, has reduced affinity for the MHCI binding pocket, and thus is replaced by a peptide of interest (darker solid circle) that may be a patient predicted epitope.

FIGS. 17A and 17B show examples of SEC-MS quantitation of a free HLA and MHCI complexes with no bound peptide, with an exchangeable peptide (with two-color circle), and with a peptide of interest after peptide exchange (with light colored circle). FIG. 17A shows quantitation of HLA and intact MHCI complexes, while FIG. 17B shows signals from MHCI complexes before and after peptide exchange.

FIGS. 18A-18D show MS analyses after SEC and CZE separations. FIGS. 18A and 18B show resolution of peptide complexes at different concentrations by MS following SEC separation. The injection volume was 4 μL. FIG. 18C shows resolution of an exemplary peptide complex by MS following CZE separation on a ZipChip™ CZE device. Injection was at 100 μg/mL in a 3 nL injection volume. FIG. 18D shows MS analysis of an exemplary exchangeable peptide before UV exposure (top; two color circle) and an exemplary exchanged peptide of interest after UV exposure (bottom, solid circle) following CZE separation.

FIGS. 19A and 19B show exemplary data in which percentage peptide exchange was assessed for a number of different exemplary peptides from SEC-native MS analysis. As shown in FIG. 19A, the exchange was first assessed to ensure saturation. The fraction of exchangeable peptide exchanged for peptide of interest is shown in FIG. 19B.

FIG. 20 shows a workflow schematic of the production and assembly of MHCI tetramers for use in immune monitoring of patient T-cells.

FIG. 21 shows a cartoon schematic of methods for assessing the exchange profile of a UV-cleavable peptide for a second peptide of interest for recombinant MHCI complexes, including ELISA, TR-FRET, and 2D-LC/MS.

FIG. 22 shows a workflow schematic of measuring intact MHCI complexes using SEC-MS, and two example mass spectra.

FIG. 23 shows an example native mass spectrum, a plot of relative abundance vs. m/z (mass-to-charge) of various native MHCI species with and without peptide bound. Each set of charge states in the spectrum is labeled with a cartoon representation of the corresponding MHCI complex, with or without a peptide bound.

FIG. 24 shows a magnified view of a single charge state of an example native mass spectrum plot of a MHCI complex with a peptide bound. Multiple peaks indicate different charge states, which correspond to different MHCI complex with and without peptide, or with and without buffer adducts, or with or without a starting methionine.

FIG. 25 shows example mass spectra of two different time points of the same peptide exchange reaction. The corresponding MHCI complex with or without peptide bound is labeled above each peak.

FIG. 26 shows two example mass spectra used for quantitation of intact MHCI complex post peptide exchange time course. The peaks in each spectrum are labeled with a cartoon representation of the MHCI species present. The deconvoluted spectrum on the right shows MHCI complex bound with either the UV-cleavable peptide or the exchanged peptide post-exchange time course.

FIG. 27 is a schematic showing three mass spectrometry (MS) based methods for monitoring a MHCI complex and peptide exchange reaction, initiated by UV-light exposure, by SEC-MS. The components of each sample are determined by high resolution MS (HR-MS), native MHCI complex composition analysis is determined by native MS, and binding affinities between peptides and MHCI complexes are determined by measuring % exchange over time by native MS.

FIG. 28 is a cartoon representation for high-throughput screening schematic of both different MHCI HLA alleles and different peptides at once. After patient-derived T-cells have been sorted from PBMC samples, they can be further tested in a high-throughput format for responsiveness to 1000s of different combinations of MHCI HLA alleles/predicted peptide epitopes ex vivo.

FIG. 29 shows mass spectra over a concentration range of HLA-A*02:01 MHCI complex with and without UV-cleavable peptide bound. The range of concentrations injected onto the SEC-equipped uHPLC are from 2.5 mg/mL to 83 μg/mL, and the injection volume was 4 μL for each injection. The protein species of interest, the MHCI complex with or without peptide bound, co-elute as a single peak in the SEC chromatogram (in plots of intensity vs. time, highlighted). Each of the possible protein species are indicated as cartoons above the corresponding peaks in the mass spectra corresponding to the single SEC chromatogram peak.

FIG. 30 shows multiple electropherograms and the corresponding MS1 mass spectra of repeat 3 nL injections of 0.1 mg/mL HLA-A*02:01 MHCI complex on an HSN ZipChip-equipped mass spectrometer.

FIG. 31 shows multiple electropherograms and the corresponding MS1 mass spectra of repeat 3 nL injections of either 41.0 or 20.5 μg/mL HLA-A*02:01 MHCI complex on an HSBG ZipChip-equipped mass spectrometer.

FIG. 32 shows mass spectra collected at various cap temperatures and ESI sheath gas source settings to optimize instrument conditions for MHCI complex analysis.

FIG. 33 shows a comparison between SEC-MS and CZE-MS. The top row, from left to right, shows a representative chromatogram, MS1 spectrum, and deconvoluted peak spectrum for HLA-A*01:01 from SEC-MS analysis. The bottom row, from left to right, shows a representative electropherogram, MS1 spectrum, and deconvoluted peak spectrum for HLA-A*01:01 from CZE-MS analysis.

FIG. 34 shows repeat electropherograms and the corresponding deconvoluted mass spectra for a range of protein concentrations of HLA-A*01:01. For each run, 3 nL of protein was injected onto the HSBG chip at a voltage of 500 V/cm. The sample concentration range was 41-2.05 μg/mL.

FIG. 35 shows two methods for buffer exchanging a protein sample prior to analysis, by desalting spin column or by spin desalting plate.

FIG. 36 shows the resulting deconvoluted mass spectra of MHCI protein complexes before and after UV-light initiated peptide exchange reactions. The MHCI species composition in each peak is indicated by a cartoon label.

FIG. 37 shows two different bar graph representations of % completeness (y-axis) for MHCI complex formation and for MHCI/peptide exchange reaction for a number of different exchange peptides (indicated by the number labeled on the x-axis, one per bar) for four different MHCI alleles (indicated below each grouping of bars). The top row shows the relative amounts of free HLA vs. MHCI complex. The bottom row shows the relative amounts of MHCI complex with UV-cleavable peptide bound (UV-MHCI) or exchanged peptide bound (pMHCI).

FIG. 38 shows a cartoon schematic of the MHCI complex structure including the ct chain and B2M, as well as deconvoluted mass spectra of the MHCI complex with and without the starting methionine, and with purification adducts, analyzed on an Extended Mass Range (EMR) Exactive Orbitrap (grey spectrum), or on the Orbitrap Eclipse (black spectrum), demonstrating the gas phase dissociation of the peptide on only the EMR analysis. A cartoon representation of each MHCI complex species is shown next to the corresponding MS peak. A star label indicates the starting methionine is present. An asterisk label indicates additional mass observed for the purification adduct.

FIG. 39 shows MS1 spectra of MHCI complex (red charge labels) when analyzed at increasing voltages on the Orbitrap Eclipse. As source voltage is increased, the peptide can be dissociated from the complex and de novo sequenced independently, with the HLA/B2M complex remaining (green charge labels).

FIG. 40 shows MS1 spectra of the MHCI complex components after gas phase dissociation. Free peptide is shown in the insert.

FIGS. 41A-41C show ELISA assay development. FIG. 41A is the comparison of ELISA formats. CMV pp65 signal normalized to no peptide control at 0.03-6.67 μg/mL. The format with anti-B2M as coat and anti-HLA-biotin as detection showed less differential between CMV pp65 and no peptide control at 0.74, 2.22, and 6.67 μg/mL compared to the anti-HLA coat and anti-B2M detection. FIG. 41B shows the ELISA analysis of HLA-A*02:01 after small scale refold with CMV pp65, BMRF1 and no peptide. ELISA analysis was run at MHCI concentrations ranging from 0.005 to 3.33 μg/mL. FIG. 41C shows ELISA OD values for the CMV pp65 and BMFR1 peptides normalized to the no peptide control at 1 μg/mL.

FIGS. 42A-42H show the process of identification of candidate conditional MHCI ligands for A*02:03, B*35:03 and C*02:02. FIG. 42A shows OD values for the five peptides screen with A*02:03 at various concentrations (0.1-3 μg/mL). Normalized ELISA OD for FIG. 42B, A*02:03, FIG. 42C, B*35:03, and FIG. 42D, C*02:02, MHC complexes with selected peptides at 1 μg/mL. The peptide yielding the highest normalized OD value for each HLA was selected and variants were designed with UV-amino acid substitutions at positions 2, 4, 6 and 8 from the N-term. FIG. 42E shows the OD values for the four conditional MHCI ligands derived from peptide A02:03-02 screened with HLA-A*02:03 at various concentrations. Normalized ELISA OD for FIG. 42F, HLA A*02:03, FIG. 42G, B*35:03-05, and FIG. 42H, C*02:02-03, MHC complexes with the selected conditional MHCI ligands derived from the peptides identified in FIGS. 42B, 42C, and 42D. Peptides not containing the engineered J amino acids (parent peptide) were used as internal controls (gray bars). For all assays, the no peptide (NP) was used as a negative control.

FIGS. 43A-43E show the biotinylation analysis, purification, and characterization of scaled-up, refolded A0201 MHCI material. FIG. 43A is the LC/MS analysis of the HLA allele in the refolded MHCI reaction mixture before (black line) and after (gray line) biotinylation. The two peaks correspond to the full-length HLA and truncated HLA with N-terminal cleavage of the methionine. The shift in both peaks after biotinylation corresponds to the molecular weight (MW) of biotin. FIG. 43B shows an anion exchange chromatogram of the biotinylated MHCI complex after refold and SDS-PAGE analysis of the fractions collected in the highlighted box. The MW of the SDS-PAGE bands correspond to B2M and HLA. FIG. 43C shows an LC/MS TIC chromatogram of the purified MHCI complex. The peaks at 1.625 and 1.74 min correspond to the UV-peptide and the peaks at 1.8 and 2.2 min correspond to B2M and HLA, respectively. FIG. 43D shows SEC-MALS analysis of the purified MHCI complex. The black line corresponds to the A280 chromatogram (left y axis) and the dashed line corresponds to the MW analysis (right y axis). FIG. 43E shows the MHCI yields after purification at the 1, 5 and 15 L scale.

FIGS. 44A-44D show the scale up production, purification and characterization of conditional MHCI complexes. FIG. 44A is the SDS-PAGE analysis, FIG. 44B is the refold yield, FIG. 44C shows the B2M to HLA ratio and FIG. 44D is the SEC-MALS MW analysis of purified refolded MHCI complexes generated with the conditional MHCI ligands identified in the small scale screen.

FIGS. 45A-45J show the 2D LC/MS analysis of peptide exchange. FIG. 45A shows a schematic of the 2D LC/MS workflow. FIG. 45B shows an A280 nm SEC chromatogram of the 1^{st} dimension in the 2D LC/MS analysis of HLA-A*02:01 MHCI complex after exchange with CMV pp65. FIG. 45C shows an A280 nm SEC chromatogram of the 2^{nd} dimension in the 2D LC/MS analysis of HLA-A*02:01 MHCI complex after exchange with CMV pp65. FIG. 45D shows an EIC chromatogram of the exchange peptide (black line) and conditional MHCI ligand (dashed line) in the 2^{nd} dimension in the 2D LC/MS analysis of HLA-A*02:01 MHCI complex after exchange with CMV pp65. FIG. 45E shows an A280 nm SEC chromatogram of the first dimension in the 2D LC/MS analysis of A*02:03 MHCI complex after exchange with A0203-05 peptide. FIG. 45F shows an A280 nm SEC chromatogram of the 2^{nd} dimension in the 2D LC/MS analysis of A*02:03 MHCI complex after exchange with A0203-05 peptide. FIG. 45G shows an EIC chromatogram of the exchange peptide (black line) and conditional MHCI ligand (dashed line) in the 2^{nd} dimension in the 2D LC/MS analysis of HLA-A*02:03 MHCI complex after exchange with A0203-05 peptide. FIG. 45H shows an A280 nm SEC chromatogram of the 1^{st} dimension in the 2D LC/MS analysis of A0*02:03 MHCI complex after exchange with a known non-binding peptide. FIG. 45I shows an A280 nm SEC chromatogram of the 2^{nd} dimension in the 2D LC/MS analysis of A*02:03 MHCI complex after exchange with non-binding peptide. FIG. 45J shows an EIC chromatogram of the exchange peptide (black line) and conditional MHCI ligand (dashed line) in the 2nd dimension in the 2D LC/MS analysis of HLA-A*02:03 MHCI complex after exchange with irrelevant peptide.

FIGS. 46A-46F show the quantification of the 1^{st} dimension A280 MHCI peak after peptide exchange in the 2D LC/MS analysis. MHCI peak area after peptide exchange normalized by peak area before peptide exchange for A*02:03, FIG. 46A, A*26:01, FIG. 46B, B*18:01, FIG. 46C, B*35:03, FIG. 46D, C*02:02, FIG. 46E, and C*14:02, FIG. 46F, for positive control peptides (known binders, black bars) and non-binder peptide (gray bar). The positive symbol indicates the exchange peptide was observed in the EIC analysis of the second dimension and a negative symbol indicates the exchange peptide was not observed in the EIC analysis.

FIGS. 47A-47C shows ELISA assay development. FIG. 47A, comparison of ELISA formats. S/N values at MHCI concentration of 0.03-6.67 μg/mL for CMV pp65 and HLA-A*02:01. FIG. 47B, ELISA analysis of HLA-A*02:01 after small scale refold with CMV pp65, BMRF1 and no peptide for ELISA format 2. ELISA analysis was run at MHCI concentrations ranging from 0.005 to 3.33 μg/mL. FIG. 47C, ELISA S/N values for ELISA Format 2 with MHCI complexes assembled with CMV pp65 and BMFR1 peptides and HLA-A*02:01 at an MHCI concentration of 1 μg/mL.

FIGS. 48A-48H shows the identification of candidate conditional MHCI ligands for A*02:03, B*35:03 and C*02:02. FIG. 48A, OD values for the five peptides screened with A*02:03 at various concentrations (0.1-3 μg/mL). S/N ELISA values for FIG. 48B, A*02:03, FIG. 48C, B*35:03 and FIG. 48D, C*02:02 MHC complexes with selected peptides at 1 μg/mL. FIG. 48E, OD values for the four conditional MHCI ligands derived from peptide A02:03-02 screened with HLA-A*02:03 at various concentrations. S/N values for FIG. 48F, HLA A*02:03, FIG. 48G, B*35:03-05 and FIG. 48H, C*02:02-03 MHC complexes with the selected conditional MHCI ligands derived from the peptides identified in FIGS. 48B, 48C, and 48D. Peptides not containing the engineered J amino acids (parent peptide) were used as internal controls (gray bars). For all assays, the no peptide (NP) was used as a negative control.

FIG. 49 shows a schematic of scaled-up production of A*02:01 MHCI monomer. The first step in the protocol developed for scaled-up production of MHCI complexes is to mix all refold components and allow the refold to occur. The second step is in-process biotinylation, followed by anion exchange chromatography in the third step.

FIGS. 50A-50F Biotinylation analysis, purification, and characterization of scaled-up, refolded A*02:01 MHCI monomer. FIG. 50A, LC/MS analysis of the HLA allele in the refolded MHCI reaction mixture before (black line) and after (gray line) biotinylation. The two peaks correspond to the full-length HLA and truncated HLA with N-terminal cleavage of the methionine. The shift in both peaks after biotinylation corresponds to the MW of biotin. FIG. 50B, anion exchange chromatogram of the biotinylated MHCI complex after refold and SDS-PAGE analysis of the fractions collected in the highlighted box (FIG. 50C). The MW of the SDS-PAGE bands correspond to B2M (13 kDa) and HLA (37 kDa). FIG. 50D, LC/MS TIC chromatogram of the purified MHCI complex. The peaks at 1.625 and 1.74 min correspond to the UV-peptide and the peaks at 1.8 and 2.2 min correspond to B2M and HLA, respectively. FIG. 50E, SEC-MALS analysis of the purified MHCI complex. The black line corresponds to the A280 chromatogram (lefty axis) and the dashed line corresponds to the MW analysis (right y axis). FIG. 50F, MHCI % yields (mg purified MHCI/mg MHCI in refold*100)±standard deviation (N=3) after purification at the 1, 5, and 15 L scale.

FIGS. 51A-51D show a scale up production, purification and characterization of conditional MHCI complexes. FIG. 51A, SDS-PAGE analysis, FIG. 51B, average refold % yield (mg purified MHCI/mg MHCI in refold*100)±standard deviation (N=3), FIG. 51C, average B2M to HLA ratio±standard deviation (N=3) and FIG. 51D, average SEC-MALS MW analysis±standard deviation (N=3) of purified refolded MHCI complexes generated with the conditional MHCI ligands identified in the small scale screen.

FIGS. 52A-52J show 2D LC/MS analysis of peptide exchange. FIG. 52A, schematic of the 2D LC/MS workflow. FIG. 52B, A280 nm SEC chromatogram of the first dimension in the 2D LC/MS analysis of HLA-A*02:01 MHCI complex after exchange with CMV pp65. The dotted line defines the region that was collected and injected into the second column. FIG. 52C, A280 nm SEC chromatogram of the second dimension in the 2D LC/MS analysis of HLA-A*02:01 MHCI complex after exchange with CMV pp65. FIG. 52D, EIC chromatogram of the exchange peptide (black line) and conditional MHCI ligand (dashed line) in the second dimension in the 2D LC/MS analysis of HLA-A*02:01 MHCI complex after exchange with CMV pp65. FIG. 52E, A280 nm SEC chromatogram of the first dimension in the 2D LC/MS analysis of A*02:03 MHCI complex after exchange with A0203-05 peptide. The dotted line defines the region that was collected and injected into the second column. FIG. 52F, A280 nm SEC chromatogram of the second dimension in the 2D LC/MS analysis of A*02:03 MHCI complex after exchange with A0203-05 peptide. FIG. 52G, EIC chromatogram of the exchange peptide (black line) and conditional MHCI ligand (dashed line) in the second dimension in the 2D LC/MS analysis of HLA-A*02:03 MHCI complex after exchange with A0203-05 peptide. FIG. 52H, A280 nm SEC chromatogram of the first dimension in the 2D LC/MS analysis of A0*02:03 MHCI complex after exchange with a known nonbinding peptide. The dotted line defines the region that was collected and injected into the second column. FIG. 52I, A280 nm SEC chromatogram of the second dimension in the 2D LC/MS analysis of A*02:03 MHCI complex after exchange with nonbinding peptide. FIG. 52J, EIC chromatogram of the exchange peptide (black line) and conditional MHCI ligand (dashed line) in the second dimension in the 2D LC/MS analysis of HLA-A*02:03 MHCI complex after exchange with irrelevant peptide.

FIGS. 53A-53F show the quantification of the first dimension A280 MHCI peak after peptide exchange in the 2D LC/MS analysis. Fraction of MHCI peak area after peptide exchange relative to the peak area before peptide exchange for FIG. 53A, A*02:03, FIG. 53B, A*26:01, FIG. 53C, B*18:01, FIG. 53D, B*35:03, FIG. 53E, C*02:02, and FIG. 53F, C*14:02 for positive control peptides (known binders, black bars) and non-binder peptide (gray bar). The positive symbol indicates the exchange peptide was observed in the EIC analysis of the second dimension and a negative symbol indicates the exchange peptide was not observed in the EIC analysis.

FIGS. 54A-54F show the identification of conditional MHCI ligand for A*26:01, B*18:01 and C*14:02 HLA alleles. FIG. 54A, A*26:01, FIG. 54B, B*18:01 and FIG. MC, C*14:02 MHC complexes with selected peptides at 1 μg/mL. The peptide yielding the highest normalized OD value for each HLA was selected and variants were designed with UV-amino acid substitutions at positions 2, 4, 6 and 8 from the N-terminus. Normalized ELISA OD for A*26:01 (FIG. 54D), B*18:01 (FIG. 54E) and C*14:02 (FIG. 54F) MHC complexes with the selected conditional MHCI ligands derived from the peptides identified in FIGS. 54A, 54B, and 54C. Peptides not containing the engineered J amino acids (parent peptides) were used as internal controls (gray bar). For all assays, the no peptide (NP) was used as a negative control.

FIGS. 55A-55F show the anion exchange chromatogram of the biotinylated MHCI complex after refold and SDS-PAGE analysis of the fractions collected in the highlighted box for A*02:03 (FIG. 55A), A*26:01 (FIG. 55B), B*18:01 (FIG. 55C), B*35:03 (FIG. 55D), C*02:02 (FIG. 55E) and C*14:02 (FIG. 55F).

### DETAILED DESCRIPTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.

### Definitions

Unless defined otherwise, all technical and scientific terms used herein have the same meaning as commonly understood by one of ordinary skill in the art to which this disclosure belongs. In this specification and in the claims that follow, reference will be made to a number of terms that shall be defined to have the following meanings:

As described herein, any concentration range, percentage range, ratio range or integer range is to be understood to include the value of any integer within the recited range and, when appropriate, fractions thereof (such as one tenth and one hundredth of an integer), unless otherwise indicated.

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

“Selective” or “selectivity” or the like of a compound refers to the compound's ability to discriminate between molecular targets (e.g. a compound having selectivity toward HMT SUV39H1 and/or HMT G9a).

“Specific”, “specifically”, “specificity”, or the like of a compound refers to the compound's ability to cause a particular action, such as inhibition, to a particular molecular target with minimal or no action to other proteins in the cell (e.g. a compound having specificity towards HMT SUV39H1 and/or HMT G9a displays inhibition of the activity of those HMTs whereas the same compound displays little-to-no inhibition of other HMTs such as DOT1, EZH1, EZH2, GLP, MLL1, MLL2, MLL3, MLL4, NSD2, SET1b, SETT/9, SETS, SETMAR, SMYD2, SUV39H2).

A “sample” as used herein refers to any specimen intended for analysis. In some embodiments, a sample is taken from a patient. In some embodiments, the sample is a “biological fluid sample.” A “biological fluid sample” as used herein refers to any biological fluid from an organism or subject. Examples include whole blood, plasma, tears, saliva, lymph fluid, urine, serum, cerebral spinal fluid, pleural effusion, and ascites.

The terms “immune response” and the like refer, in the usual and customary sense, to a response by an organism that protects against disease. The response can be mounted by the innate immune system or by the adaptive immune system, as well known in the art.

The terms “modulating immune response” and the like refer to a change in the immune response of a subject as a consequence of administration of an agent, e.g., a compound as disclosed herein, including embodiments thereof. Accordingly, an immune response can be activated or deactivated as a consequence of administration of an agent, e.g., a compound as disclosed herein, including embodiments thereof.

“B Cells” or “B lymphocytes” refer to their standard use in the art. B cells are lymphocytes, a type of white blood cell (leukocyte), that develops into a plasma cell (a “mature B cell”), which produces antibodies. An “immature B cell” is a cell that can develop into a mature B cell. Generally, pro-B cells undergo immunoglobulin heavy chain rearrangement to become pro B pre B cells, and further undergo immunoglobulin light chain rearrangement to become an immature B cells. Immature B cells include T1 and T2 B cells.

“T-cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T-cells include, for example, natural killer T (NKT) cells, cytotoxic T lymphocytes (CTLs), regulatory T (Treg) cells, and T helper cells. Different types of T-cells can be distinguished by use of T-cell detection agents.

A “regulatory T-cell” or “suppressor T-cell” is a lymphocyte which modulates the immune system, maintains tolerance to self-antigens, and prevents autoimmune disease.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may, in embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain may be a non-natural amino acid side chain.

The term “non-natural amino acid side chain” refers to the functional substituent of compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid.

The term “UV-cleavable amino acid side chain” refers to the functional substituent of compounds that have the same basic chemical structure as a naturally occurring amino acid, i.e., an a carbon that is bound to a hydrogen, a carboxyl group, an amino group, and an R group. UV-cleavable amino acids are non-proteinogenic amino acids that either occur naturally or are chemically synthesized. Such analogs may have modified R groups or modified peptide backbones, but retain the same basic chemical structure as a naturally occurring amino acid. UV-cleavable amino acids include, without limitation, 2-nitrophenylglycine (NPG), expanded o-nitrobenzyl linker, o-nitrobenzylcaged phenol, o-nitrobenzyl caged thiol,32 nitroveratryloxycarbonyl (NVOC) caged aniline, o-nitrobenzyl caged selenides, bis-azobenzene, coumarin, cinnamyl, spiropyran, 2-nitrophenylalanine (2-nF), and 3-amino-3-(2-nitrophenyl)propionic acid (ANP) amino acid analogs.

The term “MHCI” or “major histocompatibility complex class I” or “major histocompatibility complex I” or “MHCI monomer” as provided herein includes any of the recombinant or naturally-occurring forms of the major histocompatibility complex-1 (MHCI) or variants or homologs thereof that maintain MHCI activity (e.g. within at least 50%, 80%, 90%, 95%, 96%, 97%, 98%, 99% or 100% activity compared to MHCI). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MHCI polypeptide. In embodiments, MHCI is a heterodimer of two non-covalently bound proteins, a heavy chain (α) and a light chain (β₂-microglobulin), homolog or functional fragment thereof. In embodiments, MHCI includes a peptide ligand.

The term “HLA” or “human leukocyte antigen” refers to a group of proteins encoded by the MHC gene complex. Specifically, but not limited to, the MHCI gene complex encodes for the HLA-A, HLA-B, and HLA-C group of proteins.

The term “Beta-2 microglobulin” or “B2M” or “β₂ microglobulin” or “beta chain” refers to the smaller, or light chain protein of the cell surface MHCI protein complex. B2M forms a heterodimeric complex with one a chain (heavy chain). B2M is encoded by the B2M gene.

The term “a chain” or “alpha chain” refers to the larger, or heavy chain protein of the MHCI protein complex. The a chain is further divided into subunits α1, α2, and α3, and contains one transmembrane helix. The a chain binds B2M via the α3 subunit to form the heterodimer known as the MHCI complex. The a chain is polymorphic, and encoded by mainly the HLA-A, HLA-B, and HLA-C genes, and to a lesser extent by HLA-E, HLA-F, HLA-G, HLA-K, and HLA-L.

The term “ligand” refers to a molecule that forms a complex with a biomolecule to serve a biological function. Binding can take place between, and not limited to, proteins, peptides, RNA, DNA, nucleic acids, nucleic acid derivatives, non-natural nucleic acids, amino acids, amino acid derivatives, non-natural amino acids, carbohydrates, monosaccharides, disaccharides, oligosaccharides, oligonucleotides, metals, metal complexes, drugs, lipids, fatty acids, metabolites, inorganic molecules, organic molecules, biopolymers, and polymers. Ligand complexes can be formed via ionic bonding, covalent bonding, Van der Waals interactions, and/or hydrogen bonding. Ligands for MHCI are generally peptides.

The terms “bind” and “bound” as used herein is used in accordance with its plain and ordinary meaning and refers to the association between atoms or molecules. The association can be direct or indirect. For example, bound atoms or molecules may be direct, e.g., by covalent bond or linker (e.g. a first linker or second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

The term “denature” refers to a process where the three-dimensional structure of a protein, polypeptide, DNA, RNA, or other biopolymer is disrupted by chemical or mechanical means, or by heating or cooling.

The process of “peptide-exchange” refers to first forming an MHCI complex bound to a peptide that is capable of being replaced by, or exchanged for, another peptide of analytical interest such as a putative neoantigen peptide. In some cases, exchange can be promoted by decreasing the binding affinity of the first peptide for the peptide of interest, such as through chemical, enzymatic, or UV-mediated cleavage of the first peptide.

A “neoantigen” or “neoantigen peptide” refers to a peptide that may be recognized by the host's immune system as “non-self” Neoantigen peptides may be derived from mutant proteins, for example, in tumor cells. They may also be derived from pathogenic proteins from viruses or bacteria or other pathogens. Or they may also be derived from grafts such as tissue grafts or allografts or other transplanted cells.

A “1D-LC” or “one-dimensional liquid chromatography” process refers to a single liquid chromatography separation, in contrast to a “2D-LC” or “two-dimensional liquid chromatography,” which refers to a method of chromatography in which two separations are performed.

“Size exclusion chromatography” or “SEC,” is a means of chromatography in which molecules are separated by size on a solid phase chromatography medium, with larger molecules travelling through the solid phase column at a different rate than smaller molecules. In some embodiments, SEC is used in a 1D-LC process. SEC could also be used in a 2D-LC process combined with a different form of separation, such as a reversed-phase liquid chromatography step, or an ion exchange or cation exchange or affinity separation, may be employed as a second dimension.

“Capillary electrophoresis” or “CE” refers to a process in which an electric current is used to move molecules through a capillary. Each molecule's mobility may depend on its charge, size, and shape. There are several types of CE, including capillary zone electrophoresis (CZE), capillary gel electrophoresis (CGE), micellar electrokinetic capillary chromatography (MEKC), capillary electrochromatography (CEC), capillary isoelectric focusing (CIEF), and capillary isoelectrophoresis (CITP), among others.

“Capillary zone electrophoresis” or “CZE” as used herein refers to a type of CE in which different molecules in a buffer solution can be separated based on their different mobilities.

“Mass spectrometry” or “MS” refers to a technique that measures the mass to charge ratio (m/z) of one or more molecules in a sample. As used herein, “tandem MS” or “MS/MS” refers to the process by which a single ion, multiple ions, or the entire mass envelope (the precursor(s)) are moved to a fragmentation chamber and the fragmented products are then sent to a mass analyzer. Depending on the design of the mass spectrometer, the fragmentation event can happen before a single mass analyzer, between two or multiple different analyzers, or within a single mass analyzer.

MS analysis may have a variety of options. In some embodiments, the MS instrument does not comprise a quadrupole. In some embodiments, the MS instrument comprises at least one quadrupole. In some embodiments, the MS instrument comprises at least 2 quadrupole analyzers. In some cases, the MS instrument comprises an octopole. In some embodiments, the MS instrument comprises at least 3 quadrupole analyzers. In some MS's, the detector is an ion trap, quadrupole, orbitrap, or TOF. In some embodiments, the MS instrument or method is multiple reaction monitoring (MRM), single ion monitoring (SIM), triple stage quadrupole (TSQ), quadrupole/time of flight (QTOF), quadrupole linear ion trap (QTRAP), hybrid ion trap/FTMS, time of flight/time of flight (TOF/TOF), Orbitrap instruments, ion trap instruments, parallel reaction monitoring (PRM), data dependent acquisition (DDA), data independent acquisition (DIA), multi-stage fragmentation or tandem in time MS/MS. In some embodiments, an electrospray, Orbitrap instrument is used.

“Native mass spectrometry” is an MS process that is performed on a molecule in its native state, i.e., wherein the molecule is not unfolded or denatured.

As used herein, the abbreviations “SEC-native MS” or “SEC-MS” refer to an SEC followed by native MS process. As used herein, the abbreviations “CE-native MS,” “CZE-native MS,” “CE-MS,” and “CZE-MS” refer to a capillary electrophoresis (CE) or capillary zone electrophoresis (CZE) followed by native MS process.

The term “quantitation” or “quantitate” means herein to determine numerically the level or amount or number or concentration of an analyte in the sample.

In general, a “subject” as referred to herein is an individual whose biological sample is to be tested for presence of an analyte. In some embodiments, the subject is a human. However, in some embodiments, the subject may also be another mammal, such as a domestic or livestock species, e.g., dog, cat, rabbit, horse, pig, cow, goat, sheep, etc., or a laboratory animal, such as a mouse or rat. Mammals include, but are not limited to, domesticated animals (e.g., cows, sheep, cats, dogs, and horses), primates (e.g., humans and non-human primates such as monkeys), rabbits, and rodents (e.g., mice and rats), for example.

As used herein, an “automated” or “automatically controlled” process is one that is capable of being run, for example, by a computerized control system with appropriate software, as opposed to a system that requires an active, manual intervention during or between at least one step, such as to move an analyte-containing sample from one part of the system to another.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

One skilled in the art would understand that descriptions of making and using the complexes described herein is for the sole purpose of illustration, and that the present disclosure is not limited by this illustration.

The publications discussed herein are provided solely for their disclosure prior to the filing date of the present application. Nothing herein is to be construed as an admission that such publications constitute prior art to the claims appended hereto. All publications referenced herein are incorporated herein by reference in their entireties for all of their teachings, including, but not limited to, all compositions, components, reagents, and methods.

### MHCI Compositions

In an aspect, provided herein, is a major histocompatibility complex class I (MHCI)/ligand complex including a MHCI molecule, which includes an alpha chain, a beta chain, and a ligand, wherein the ligand is a peptide comprising a non-natural UV-cleavable amino acid.

In embodiments, the MHCI/ligand complex contains an alpha chain, where the alpha chain is encoded by any one of the following loci: HLA-A, HLA-B, or HLA-C. In some embodiments, the alpha chain is encoded by the HLA-A loci. In some embodiments, the alpha chain is encoded by the HLA-B loci. In some embodiments, the alpha chain is encoded by the HLA-C loci.

In embodiments, the MHCI/ligand complex contains a beta-2 microglobulin domain (B2M), where the B2M domain is encoded by MHCI gene complex.

In embodiments, the MHCI/ligand complex contains a peptide ligand. In embodiments, the peptide ligand is between 8 and 11 amino acid residues in length. In some embodiments, the peptide ligand is 8 amino acid residues in length. In some embodiments, the peptide ligand is 9 amino acid residues in length. In some embodiments, the peptide ligand is 10 amino acid residues in length. In some embodiments, the peptide ligand is 11 amino acid residues in length.

In embodiments, the MHCI/ligand complex contains a peptide ligand, wherein the peptide ligand contains a non-natural amino acid. In some embodiments, the non-natural amino acid is activated by UV radiation. In some embodiments, the peptide ligand containing the non-natural amino acid is cleaved after irradiation by UV light. In some embodiments, the non-natural amino acid is selected from 2-nitrophenylglycine (NPG), expanded o-nitrobenzyl linker, o-nitrobenzylcaged phenol, o-nitrobenzyl caged thio1,32 nitroveratryloxycarbonyl (NVOC) caged aniline, o-nitrobenzyl caged selenides, bis-azobenzene, coumarin, cinnamyl, spiropyran, 2-nitrophenylalanine (2-nF), and 3-amino-3-(2-nitrophenyl)propionic acid (ANP) amino acid analogs. In some embodiments, the non-natural amino acid is 3-amino-3-(2-nitrophenyl)propionic acid (ANP).

In embodiments, the non-natural amino acid can be located at any position between the N- and C-termini of the peptide ligand. In some embodiments, the non-natural amino acid is located at the N-terminus of the peptide ligand. In some embodiments, the non-natural amino acid is located at the second position of the peptide ligand (i.e., second position from the N-terminus). In some embodiments, the non-natural amino acid is located at the third position of the peptide ligand. In some embodiments, the non-natural amino acid is located at the fourth position of the peptide ligand. In some embodiments, the non-natural amino acid is located at the fifth position of the peptide ligand. In some embodiments, the non-natural amino acid is located at the sixth position of the peptide ligand. In some embodiments, the non-natural amino acid is located at the seventh position of the peptide ligand. In some embodiments, the non-natural amino acid is located at the eighth position of the peptide ligand. In some embodiments, the non-natural amino acid is located at the ninth position of the peptide ligand. In some embodiments, the non-natural amino acid is located at the tenth position of the peptide ligand. In some embodiments, the non-natural amino acid is located at the C-terminus of the peptide ligand.

In embodiments, the peptide ligand is FMYJDFHFI (SEQ ID NO.: 1); FLPJDFFPSV(SEQ ID NO.: 2); FLPSDJFPSV (SEQ ID NO.: 3); FYIQMJTEL (SEQ ID NO.: 4); YVIJDLAAM (SEQ ID NO.: 5); HFFJWGTMF (SEQ ID NO.: 6); AVVSLJRLLK (SEQ ID NO.: 7); GTHJLLPFY (SEQ ID NO.: 8); AMLTAJFLR (SEQ ID NO.: 9); HLMFYJLPI (SEQ ID NO.: 10); QLFJFSPRR (SEQ ID NO.: 11); TJFFYRYGFV (SEQ ID NO.: 12); DEFJPIVQY (SEQ ID NO.: 13); RESFGJESF (SEQ ID NO.: 14); TPAJYFHVL (SEQ ID NO.: 15); AENJYVTVF (SEQ ID NO.: 16); KEVLVLWJI (SEQ ID NO.: 17); FMYEGJTPL (SEQ ID NO.: 18); FPFJLAAII (SEQ ID NO.: 19); FPIPSJWAF (SEQ ID NO.: 20); ITAAAWYJW (SEQ ID NO.: 21); LAVMGJAAW (SEQ ID NO.: 22); HLPJGVKSL (SEQ ID NO.: 23); FAAEAJKL (SEQ ID NO.: 24); GAINSJLPY (SEQ ID NO.: 25); FAIVPJLQI (SEQ ID NO.: 26); FAMJVPLLI (SEQ ID NO.: 27); ARFJDLRFV (SEQ ID NO.: 28); ANNJRLWVY (SEQ ID NO.: 29); YAAJTNFLL (SEQ ID NO.: 30); ISDSAJNMM (SEQ ID NO.: 31); WAWJFAAVL (SEQ ID NO.: 32); MMHJSTSPF (SEQ ID NO.: 33); or RTFGQJLFF (SEQ ID NO.: 34).

### Peptide Exchange Assay

In an aspect, provided herein, is a peptide exchange assay for determining binding of a major histocompatibility complex class I (MHCI) allele to a test peptide, including: providing a first mixture, containing a free test peptide and a MHCI/ligand complex that contains an alpha chain, a beta chain, and peptide ligand that contains a non-natural, ultraviolet (UV)-cleavable amino acid within its sequence; exposing the first mixture to UV light to cleave the peptide ligand at the UV-cleavable amino acid; and incubating the first mixture for a period of time to form a second mixture, containing a second MHCI complex that contains the alpha chain, the beta chain, and the test peptide; and determining whether the MHCI allele is bound to the test peptide.

In embodiments, the amount of free test peptide in the first mixture is 1:100 to 100:1 compared to the MHCI/ligand complex. In some embodiments, the amount of free test peptide in the first mixture is 1:10 to 10:1 compared to the MHCI/ligand complex. In embodiments, the amount of free test peptide in the first mixture is 1:1 to 100:1 compared to the MHCI/ligand complex. In embodiments, the amount of free test peptide in the first mixture is 10:1 to 100:1 compared to the MHCI/ligand complex. In embodiments, the amount of free test peptide in the first mixture is about 10:1 compared to the MHCI/ligand complex. The ratio may be any value or subrange within the provided ranges, including endpoints.

In embodiments, the MHCI allele binding to the test peptide is determined by measuring a level of MHCI/test peptide complex in the second mixture. In some embodiments, the MHCI complex in the assay is partially occupied by bound test peptide in the second mixture (a portion of the total MHCI complexes in the second mixture is bound by the test peptide). In some embodiments, the MHCI complex is fully occupied by bound test peptide in the second mixture (all of the total MHCI complexes in the second mixture is bound by the test peptide).

In embodiments, the level of MHCI/second peptide complex is measured by 2-dimensional liquid chromatography-mass spectrometry (2D LC/MS) of the second mixture. In some embodiments, the 2D LC/MS includes removing the free test peptide from the second mixture. In some embodiments, the free test peptide is removed by size-exclusion chromatography. In some embodiments, the free test peptide is removed by size cut-off filtration. In some embodiments, the free peptide is removed by dialysis.

In embodiments, high-performance liquid chromatography (HPLC) and mass spectrometry (MS) to distinguish the identities of the MHCI and the test peptide. In some embodiments, the second mixture is run over an HPLC (or FPLC) equipped with a size-exclusion column. In some embodiments, the HPLC (or FPLC) is equipped to collect fractions. In some embodiments, the MHCI and test peptide are identified to elute in the same HPLC fraction. In some embodiments, free test peptide elutes in fractions different from free MHCI and MHCI/test peptide complex. In some embodiments, the co-elution of MHCI and test peptide indicates that the MHCI is capable of binding the test peptide.

In embodiments, there is more than one test peptide (e.g., more than one peptide sequence) added to the first MHCI/test peptide mixture. In some embodiments, there are two or more test peptides in the first MHCI/test peptide mixture. In embodiments, there are three or more test peptides in the first MHCI/test peptide mixture. In embodiments, there are four or more test peptides in the first MHCI/test peptide mixture. In embodiments, there are five or more test peptides in the first MHCI/test peptide mixture. In embodiments, there are six or more test peptides in the first MHCI/test peptide mixture. In embodiments, there are seven or more test peptides in the first MHCI/test peptide mixture. In embodiments, there are eight or more test peptides in the first MHCI/test peptide mixture. In embodiments, there are nine or more test peptides in the first MHCI/test peptide mixture. In embodiments, there are ten or more test peptides in the first MHCI/test peptide mixture.

In embodiments, there are 10-1000 test peptides in the first MHCI/test peptide mixture. In embodiments, there are 10-500 test peptides in the first MHCI/test peptide mixture. In embodiments, there are 10-200 test peptides in the first MHCI/test peptide mixture. In embodiments, there are 10-20 test peptides in the first MHCI/test peptide mixture. In embodiments, there are 20-30 test peptides in the first MHCI/test peptide mixture. In embodiments, there are 30-40 test peptides in the first MHCI/test peptide mixture. In embodiments, there are 40-50 test peptides in the first MHCI/test peptide mixture. In embodiments, there are 50-60 test peptides in the first MHCI/test peptide mixture. In embodiments, there are 60-70 test peptides in the first MHCI/test peptide mixture. In embodiments, there are 70-80 test peptides in the first MHCI/test peptide mixture. In embodiments, there are 80-90 test peptides in the first MHCI/test peptide mixture. In embodiments, there are 90-100 test peptides in the first MHCI/test peptide mixture. In embodiments, there are 100-110 test peptides in the first MHCI/test peptide mixture. In embodiments, there are 110-120 test peptides in the first MHCI/test peptide mixture. In embodiments, there are 120-130 test peptides in the first MHCI/test peptide mixture. In embodiments, there are 130-140 test peptides in the first MHCI/test peptide mixture. In embodiments, there are 140-150 test peptides in the first MHCI/test peptide mixture. In embodiments, there are 150-200 test peptides in the first MHCI/test peptide mixture. In embodiments, there are 200-300 test peptides in the first MHCI/test peptide mixture. In embodiments, there are 300-400 test peptides in the first MHCI/test peptide mixture. In embodiments, there are 400-500 test peptides in the first MHCI/test peptide mixture. In embodiments, there are 500-600 test peptides in the first MHCI/test peptide mixture. In embodiments, there are 600-700 test peptides in the first MHCI/test peptide mixture. In embodiments, there are 700-800 test peptides in the first MHCI/test peptide mixture. In embodiments, there are 800-900 test peptides in the first MHCI/test peptide mixture. In embodiments, there are 900-1000 test peptides in the first MHCI/test peptide mixture. The number of test peptides may be any value or subrange within the provided ranges, including endpoints. The number of test peptides are only limited by the number of test peptides recognized as feasible to use in the exchange assay by one skilled in the art.

In embodiments, there is more than one test peptide co-eluting in the MHCI/peptide complex HPLC fraction.

In embodiments, mass spectrometry is used to identify the identities of MHCI and/or test peptide(s) in the second mixture. In some embodiments, the mass spectrometer is in-line with the HPLC. In some embodiments, the HPLC fractions are collected first, then analyzed by mass spectrometry. In embodiments, the free test peptide is removed before mass spectroscopic detection of the MHCI complex. In embodiments, the amount of test peptide present in a fraction or in the second mixture is quantified by mass spectrometry by comparison to an internal standard peptide.

In embodiments, the MHCI/test peptide complex is labeled. In some embodiments, the MHCI/test peptide is fluorescently labeled. In some embodiments, the MHCI/test peptide complex is labeled by contacting a fluorescently-labeled antibody. In some embodiments, the MHCI/test peptide complex is labeled by contacting a fluorescent antibody, where the fluorescent antibody is anti-HLA. In some embodiments, the MHCI/peptide complex is labeled by biotinylation of the alpha protein.

In embodiments, the level of peptide exchange is determined by contacting the labeled MHCI/peptide complex with an antibody complex containing anti-MHCI allele antibody covalently attached to a fluorescence resonance energy transfer (FRET) donor; and a FRET acceptor complex comprising a FRET acceptor conjugated to a second label, thereby forming a reaction composition; and detecting FRET emission of the second label in the reaction composition, thereby detecting formation of a stable MHCI allele, which is a proxy measure of peptide binding. In some embodiments, the first label is an anti-MHCI antibody that is anti-B2M. In some embodiments, the first label is an anti-MHCI antibody that is chelating a Europium ion. In some embodiments, the alpha protein of the MHCI/peptide complex is biotinylated. In some embodiments, the biotinylated MHCI/peptide complex binds a second label. In some embodiments, the second label is a streptavidin protein. In some embodiments, the second label is a streptavidin protein that is covalently-linked to an allophycocyanin (APC). In some embodiments, the first and second labels have a spectral overlap integral suitable for FRET when a MHCI/peptide complex containing a first and second label is present. In some embodiments, the FRET donor/acceptor pair labels include fluorescein and tetramethylrhodamine. In some embodiments, the FRET donor/acceptor pair labels include 5-({2-[(iodoacetyl)amino]ethyl}amino)naphthalene-1 sulfonic acid (IAEDANS) and fluorescein. In some embodiments the FRET donor/acceptor pair labels include (5-((2-aminoethyl)amino)naphthalene-1-sulfonic acid (EDANS) and 4-((4-(dimethylamino)phenyl)azo)benzoic acid (Dabcyl). In some embodiments, the FRET donor/acceptor pair labels include Alexa Fluor 488 and Alexa Fluor 555. In some embodiments, the donor/acceptor pair labels include Alexa Fluor 594 and Alexa Fluor 647. In some embodiments, the donor/acceptor pair labels include europium (Eu-cryptate) and allophycocyanin (XL665). In some embodiments, the donor/acceptor pair labels include terbium and fluorescein. The first and second labels may be any suitable label pairs known in the art.

In embodiments, the peptide exchange detection assay reagents and the MHCI/peptide complex are incubated for between about 1 hour and about 48 hours. In embodiments, the peptide exchange detection assay reagents and the MHCI/peptide complex are incubated for at least about 1 hour. In some embodiments, the peptide exchange detection assay reagents and the MHCI/peptide complex are incubated for at least about 5 hours. In embodiments, the peptide exchange detection assay reagents and the MHCI/peptide complex are incubated for at least about 10 hours. In embodiments, the peptide exchange detection assay reagents and the MHCI/peptide complex are incubated for at least about 12 hours. In some embodiments, the peptide exchange detection assay reagents and the MHCI/peptide complex are incubated for at least about 15 hours. In some embodiments, the peptide exchange detection assay reagents and the MHCI/peptide complex are incubated for at least about 20 hours. In some embodiments, the peptide exchange detection assay reagents and the MHCI/peptide complex are incubated for at least about 24 hours. Incubation time may be any value or subrange within the provided ranges, including endpoints.

In some embodiments, the first label contains a streptavidin protein. In some embodiments, the first label contains an anti-HLA antibody. In some embodiments, the first label contains a monobody. In some embodiments, the first label contains a partial antibody. In some embodiments, the first label contains an scFv domain. In some embodiments, the first label contains an antibody fragment.

In some embodiments, the second label is streptavidin.

In embodiments, emission from the FRET acceptor indicates binding of a test peptide to the MHCI complex. In some embodiments, the level of bound peptide is determined by time resolved (TR) FRET detection. In some embodiments, the signal from the TR-FRET acceptor label indicates the level of MHCI complex present. In some embodiments, the level of MHCI complex present indicates the presence of a MHCI/peptide complex. In some embodiments, the MHCI/peptide complex contains a test peptide. In some embodiments, the signal from FRET emission is normalized between two or more MHCI alleles. In some embodiments, the TR-FRET assay is performed at a temperature between about 4° C. and about 50° C. In some embodiments, the TR-FRET assay is performed at room temperature. In some embodiments, the TR-FRET assay is performed at about 37° C.

### Complex Detection Assay

In an aspect, provided herein, is a method of detecting binding of a major histocompatibility complex class I (MHCI) allele to a test peptide, the method comprising: providing a first composition comprising a test peptide and a MHCI/ligand complex including: a MHCI molecule with an alpha chain, a beta chain, and a ligand, wherein the ligand is a peptide that contains a non-natural ultraviolet (UV)-cleavable amino acid; exposing the first composition to UV light to cleave the ligand at the UV-cleavable amino acid; and detecting a MHCI/test peptide complex in the second mixture, thereby detecting binding of the MHCI molecule to the test peptide.

In embodiments, the level of MHCI/test peptide complex is detected and compared to a control. In some embodiments, the level of MHCI/test peptide complex is detected and compared to an internal standard. In some embodiments, the internal standard is a peptide.

In embodiments, the level of MHCI/test peptide complex is detected using 2-dimensional liquid chromatography-mass spectrometry (2D LC/MS), e.g. as described above. In some embodiments, the second mixture is transferred into a vessel suitable for analysis by 2D LC/MS. In some embodiments, free test peptide is removed from the second mixture prior to analysis by 2D LC/MS. In some embodiments, the free test peptide is removed prior to mass spectral analysis by any non-denaturing column chromatography. Examples of non-denaturing column chromatography include, but not limited to: size-exclusion, ion exchange, hydrophobic interaction, affinity, normal-phase, or reversed-phase chromatography. In some embodiments, the free test peptide is removed from the second mixture by size-exclusion chromatography prior to analysis by 2D LC/MS.

### MHCI Binding Ligand Identification Assay

In an aspect, provided herein is method of identifying a MHCI binding ligand, including: contacting a plurality of MHCI alpha chain monomers with a plurality of beta chain monomers and a ligand under conditions that allow for the formation of a MHCI/ligand complex, wherein the ligand is a peptide containing a non-natural UV-cleavable amino acid; and detecting the MHCI/ligand complex, thereby identifying a MHCI binding ligand.

In embodiments, the MHCI alpha monomers are denatured prior to the contacting step. In some embodiments, the MHCI alpha monomers are unfolded prior to the contacting step. In some embodiments, the MHCI alpha monomers are denatured using guanidine HCl, guanidine isothiocyanate, and/or urea solution. In some embodiments, the concentration of guanidine HCl or urea is 6M. In some embodiments, a reducing reagent is present in the denaturing solution. In some embodiments, a mixture of reducing reagent and oxidizing reagent is present in the denaturing solution. In some embodiments, a buffering reagent is present in the denaturing solution. In some embodiments, a salt is present in the denaturing solution. In some embodiments, a detergent is present in the denaturing solution. In some embodiments, the MHCI alpha monomers are recovered from inclusion bodies. In some embodiments, the MHCI alpha monomers are denatured prior to application to a SEC column. In some embodiments, the MHCI alpha monomers are separated under denaturing conditions on the SEC column. In some embodiments, the MHCI alpha monomers are collected and stored under denaturing conditions.

In embodiments, the denatured alpha monomers are refolded in the presence of B2M in a refolding time course. In embodiments, the denatured alpha monomers are refolded in the presence of B2M, and a peptide ligand in a refolding time course. In embodiments, the refolding time course is initiated by rapid dilution of denatured alpha chain monomers in the presence of B2M, a peptide ligand, and any one or more of a buffering reagent, a salt, a reducing reagent, an oxidizing reagent, a counterion, a chelator, and/or a detergent. In embodiments, the refolding time course is initiated by rapid dilution of denatured alpha chain monomers in the presence of B2M, a peptide ligand, and a buffering reagent. In embodiments, the refolding time course is initiated by rapid dilution of denatured alpha chain monomers in the presence of B2M, a peptide ligand, and a buffering reagent, and a salt. In embodiments, the refolding time course is initiated by rapid dilution of denatured alpha chain monomers in the presence of B2M, a peptide ligand, and a buffering reagent, a salt, and a reducing reagent. In embodiments, the refolding time course is initiated by rapid dilution of denatured alpha chain monomers in the presence of B2M, a peptide ligand, a buffering reagent, a salt, a reducing reagent, and an oxidizing reagent. In embodiments, the refolding time course is initiated by rapid dilution of denatured alpha chain monomers in the presence of B2M, a peptide ligand, and a buffering reagent, a salt, a reducing reagent, an oxidizing reagent, and a counterion. In embodiments, the refolding time course is initiated by rapid dilution of denatured alpha chain monomers in the presence of B2M, a peptide ligand, and a buffering reagent, a salt, a reducing reagent, an oxidizing reagent, a counterion, and a chelator. In embodiments, the refolding time course is initiated by rapid dilution of denatured alpha chain monomers in the presence of B2M, a peptide ligand, and a buffering reagent, a salt, a reducing reagent, an oxidizing reagent, a counterion, a chelator, and a detergent. In some embodiments, the refolding time course takes place within the well of a 96-well plate. In some embodiments, the refolding time course takes place at 4° C. In some embodiments, the buffering reagent is tris(hydroxymethyl)aminomethane-HCl (Tris HCl), pH 8.0. In some embodiments, the counterion is L-arginine. In some embodiments, the reducing agent is reduced glutathione. In some embodiments, the oxidizing reagent is oxidized glutathione. In some embodiments, the chelator is ethylenediaminetetraacetic acid (EDTA).

In embodiments, the MHCI alpha monomers, B2M, and peptide ligands are in contact in the refolding solution for between about 5 hours and about 5 days. In embodiments, the MHCI alpha monomers, beta chain monomers, and peptide ligands are in contact in the refolding solution for at least about 12 hours, 24 hours, 48 hours. Incubation time may be any value or subrange within the provided ranges, including endpoints.

In embodiments, a plurality of ligands are contacted with the MHCI alpha and beta chain monomers. For example, multiple peptide sequences may be used in a multiplex assay format.

In embodiments, detecting the MHCI/peptide ligand complex includes: binding of the MHCI/ligand complex to an anti-MHCI alpha chain antibody attached to a solid support thereby forming a bound MHCI/ligand complex; contacting the bound MHCI/ligand complex with a labeled anti-beta chain antibody, thereby forming a bound labeled MHCI/ligand complex; and detecting the bound labeled MHCI/ligand complex. In some embodiments, the free anti-beta chain antibody is removed prior to MHCI/ligand peptide complex detection.

### MHCI Allele-Ligand Combination Optimization Assay

In an aspect, provided herein, is a method for determining optimal major histocompatibility complex class I (MHCI) allele-ligand combinations, the method including: providing a plurality of MHCI alpha chain monomers purified under denaturing conditions; forming a reaction mixture by combining the plurality of MHCI alpha chain monomers, a plurality of beta chain monomers, and a ligand comprising a peptide comprising a non-natural UV-cleavable amino acid; incubating the mixture under conditions to allow formation of a MHCI/ligand complex; and determining whether the MHCI/ligand complex was formed.

In embodiments, the plurality of MHCI alpha chain monomers, plurality of beta chain monomers, and ligand are incubated for at least between about 5 hours and about 5 days. In embodiments, the plurality of MHCI alpha chain monomers, plurality of beta chain monomers, and ligand are incubated for at least about 5 hours. In embodiments, the plurality of MHCI alpha chain monomers, plurality of beta chain monomers, and ligand are incubated for at least about 10 hours. In embodiments, the plurality of MHCI alpha chain monomers, plurality of beta chain monomers, and ligand are incubated for at least about 12 hours. In embodiments, the plurality of MHCI alpha chain monomers, plurality of beta chain monomers, and ligand are incubated for at least about 24 hours. In embodiments, the plurality of MHCI alpha chain monomers, plurality of beta chain monomers, and ligand are incubated for at least about 48 hours. In embodiments, the plurality of MHCI alpha chain monomers, plurality of beta chain monomers, and ligand are incubated for at least about 72 hours. In embodiments, the plurality of MHCI alpha chain monomers, plurality of beta chain monomers, and ligand are incubated for at least about 4 days. In embodiments, the plurality of MHCI alpha chain monomers, plurality of beta chain monomers, and ligand are incubated for about 5 days. Incubation time may be any value or subrange within the provided ranges, including endpoints.

In embodiments, a plurality of ligands are screened, wherein each ligand contains an amino acid sequence, wherein the amino acid sequence of each ligand differs from the amino acid sequence of each other ligand only by the position of the UV-cleavable amino acid in the sequence.

In embodiments, the MHCI/ligand complex formation is determined by enzyme-linked immunosorbent assay (ELISA). In some embodiments, the ELISA includes: introducing the reaction mixture into a container, the container including a surface and an anti-MHCI alpha chain antibody conjugated to the surface; introducing a labeled anti-beta chain antibody comprising a detectable label into the container, such that the labeled anti-beta chain antibody binds the beta chain monomers, if present; washing to remove unbound labeled anti-beta chain antibody; and detecting the presence of the detectable label in the container.

In embodiments, the detectable label comprises biotin or a peptide tag. In embodiments, the detectable label comprises biotin. In embodiments, the detectable biotin label is visualized by introducing a streptavidin-horseradish peroxidase (HRP) conjugate into the container and determining a level of chemiluminescence upon addition of a HRP substrate. In some embodiments, the container is a multi-well plate.

In some embodiments, the detection assay is replicated to determine the optimal MHCI/ligand complex formation between two different ligands for one MHCI complex.

In embodiments, a peptide containing a non-natural UV-cleavable amino acid, wherein the peptide has an amino acid sequence of any one of SEQ ID NO.: 1 to SEQ ID NO.: 34. In some embodiments, the peptide contains the sequence SEQ ID NO.: 1. In some embodiments, the peptide contains the sequence SEQ ID NO.: 2. In some embodiments, the peptide contains the sequence SEQ ID NO.: 3. In some embodiments, the peptide contains the sequence SEQ ID NO.: 4. In some embodiments, the peptide contains the sequence SEQ ID NO.: 5. In some embodiments, the peptide contains the sequence SEQ ID NO.: 6. In some embodiments, the peptide contains the sequence SEQ ID NO.: 7. In some embodiments, the peptide contains the sequence SEQ ID NO.: 8. In some embodiments, the peptide contains the sequence SEQ ID NO.: 9. In some embodiments, the peptide contains the sequence SEQ ID NO.: 10. In some embodiments, the peptide contains the sequence SEQ ID NO.: 11. In some embodiments, the peptide contains the sequence SEQ ID NO.: 12. In some embodiments, the peptide contains the sequence SEQ ID NO.: 13. In some embodiments, the peptide contains the sequence SEQ ID NO.: 14. In some embodiments, the peptide contains the sequence SEQ ID NO.: 15. In some embodiments, the peptide contains the sequence SEQ ID NO.: 16. In some embodiments, the peptide contains the sequence SEQ ID NO.: 17. In some embodiments, the peptide contains the sequence SEQ ID NO.: 18. In some embodiments, the peptide contains the sequence SEQ ID NO.: 19. In some embodiments, the peptide contains the sequence SEQ ID NO.: 20. In some embodiments, the peptide contains the sequence SEQ ID NO.: 21. In some embodiments, the peptide contains the sequence SEQ ID NO.: 22. In some embodiments, the peptide contains the sequence SEQ ID NO.: 23. In some embodiments, the peptide contains the sequence SEQ ID NO.: 24. In some embodiments, the peptide contains the sequence SEQ ID NO.: 25. In some embodiments, the peptide contains the sequence SEQ ID NO.: 26. In some embodiments, the peptide contains the sequence SEQ ID NO.: 27. In some embodiments, the peptide contains the sequence SEQ ID NO.: 28. V In some embodiments, the peptide contains the sequence SEQ ID NO.: 29. V In some embodiments, the peptide contains the sequence SEQ ID NO.: 30. In some embodiments, the peptide contains the sequence SEQ ID NO.: 31. In some embodiments, the peptide contains the sequence SEQ ID NO.: 32. In some embodiments, the peptide contains the sequence SEQ ID NO.: 33. In some embodiments, the peptide contains the sequence SEQ ID NO.: 34.

### Native SEC-MS and CE-MS Methods

The present disclosure relates to methods for monitoring peptide-exchanged MHCI complexes using either size exclusion chromatography or capillary electrophoresis coupled with native mass spectroscopy. One exemplary method of monitoring peptide-exchanged major histocompatibility class I (MHCI) complexes in a sample comprises: (a) obtaining peptide-exchanged MHCI complexes comprising a peptide of interest; (b) performing size exclusion chromatography (SEC), capillary electrophoresis (CE), or capillary zone electrophoresis (CZE) on the peptide exchanged MHCI complexes; and (c) following the chromatography or capillary electrophoresis of (b), performing native mass spectrometry (MS) on the MHCI complexes to identify MHCI complexes that comprise peptides of interest. Another exemplary method herein of monitoring peptide-exchanged major histocompatibility class I (MHCI) complexes in a sample comprises: (a) obtaining MHCI complexes comprising an exchangeable peptide and exposing the complexes to one or more peptides of interest under conditions which allow for peptide exchange between the exchangeable peptide and the peptide of interest; (b) performing size exclusion chromatography (SEC), capillary electrophoresis (CE), or capillary zone electrophoresis (CZE) on the peptide exchanged MHCI complexes; and (c) following the chromatography or capillary electrophoresis of (b), performing native mass spectrometry (MS) on the MHCI complexes to identify MHCI complexes that comprise peptides of interest.

Methods herein also include, for example, monitoring T-cell recognition of MHCI-complexed peptides, comprising: (a) obtaining peptide-exchanged MHCI complexes comprising a peptide of interest; (b) contacting the peptide-exchanged MHCI complexes with a sample comprising T-cells; (c) separating T-cell bound MHCI complexes from unbound MHCI complexes; (d) performing size exclusion chromatography (SEC), capillary electrophoresis (CE), or capillary zone electrophoresis (CZE) on the peptide exchanged MHCI complexes; and (e) following the chromatography or capillary electrophoresis of (d), performing native mass spectrometry (MS) on the MHCI complexes to identify MHCI complexes comprising peptides recognized by T-cells from the sample. Methods herein also include, for example, monitoring T-cell recognition of MHCI-complexed peptides, comprising: (a) obtaining major histocompatibility class I (MHCI) complexes comprising an exchangeable peptide and exposing the complexes to one or more peptides of interest under conditions which allow for peptide exchange; (b) contacting the peptide-exchanged MHCI complexes with a sample comprising T-cells; (c) separating T-cell bound MHCI complexes from unbound MHCI complexes; (d) performing size exclusion chromatography (SEC), capillary electrophoresis (CE), or capillary zone electrophoresis (CZE) on the peptide exchanged MHCI complexes; and (e) following the chromatography or capillary electrophoresis of (d), performing native mass spectrometry (MS) on the MHCI complexes to identify MHCI complexes comprising peptides recognized by T-cells from the sample. In some cases, separating T-cell bound MHCI complexes from unbound MHCI complexes is by flow cytometry.

In some methods herein, the sample is a biologic fluid sample. In some cases, the sample is a whole blood or plasma sample. In some embodiments, the sample comprises one or more synthetically produced peptides of interest. In some methods herein, the MHCI complexes are human MHCI complexes. In some methods herein, the sample is from an MHCI library or array.

In some embodiments, the method comprises performing SEC on the peptide-exchanged MHCI complexes. In some such cases, a volume of 2-10 μL, is injected for native MS analysis, such as 3-6 μL, or 4-5 μL. In some embodiments, the native MS directly follows the SEC.

In some embodiments, the method comprises performing CE on the peptide-exchanged MHCI complexes. In some embodiments, the method comprises performing CZE on the peptide-exchanged MHCI complexes. In some cases, the exchanged peptide is detectable at a concentration of 100 μg/mL or lower, 50-500 μg/mL, 50-200 μg/mL, 100-200 μg/mL, or 50-100 μg/mL. In some cases in which the method uses CE or CZE, a volume of 2-100 nl is injected for native MS analysis, such as 2-50 nl, 2-10 nl, 3-10 nl, or 3-5 nl. In some embodiments, the native MS directly follows the CE or CZE.

In some cases, methods herein allow for determination and quantitation of the degree to which the at least one peptide of interest has exchanged into the MHCI complex. Thus, they may allow for monitoring a peptide exchange reaction and/or for determining the percent or degree of exchange once the reaction has reached its maximum extent.

In some embodiments, the native MS further comprises characterizing the structure or sequence of the peptide of interest bound to the MHCI complex. In some cases, the native MS is performed as a tandem MS (“MS/MS”) (such as via multiple reaction monitoring (MRM), single ion monitoring (SIM), triple stage quadrupole (TSQ), quadrupole/time of flight (QTOF), quadrupole linear ion trap (QTRAP), hybrid ion trap/FTMS, time of flight/time of flight (TOF/TOF), or tandem in time MS/MS). In some cases, the native MS comprises electrospray ionization into an orbitrap MS instrument.

In some methods above, the chromatography or electrophoresis and native MS are performed in an ammonium acetate or ammonium folate buffer and/or wherein the buffer does not comprise TRIS or PBS.

Methods herein include, for example, methods of performing native mass spectrometry (MS) on peptide-exchanged major histocompatibility class I (MHCI) complexes in a sample. In some cases, the methods comprise: (a) obtaining peptide-exchanged MHCI complexes comprising a peptide of interest; (b) performing size exclusion chromatography (SEC) or performing capillary electrophoresis (CE), such as capillary zone electrophoresis (CZE), on the peptide exchanged MHCI complexes; and (c) following the chromatography or capillary electrophoresis of (b), performing native mass spectrometry (MS) on the MHCI complexes to identify MHCI complexes that comprise peptides of interest. In some embodiments, SEC is used in step (b). In some embodiments, CE is used in step (b). In some cases, CZE is used in step (b). In some embodiments, no further (i.e. no second dimension) chromatography or other separation process is performed between the SEC, CE or CZE and the MS analysis. Thus, in some embodiments, the native MS directly follows the SEC, CE or CZE without further wash, buffer exchange, or chromatography steps.

Methods herein also include, for example, a method of monitoring peptide-exchanged major histocompatibility class I (MHCI) complexes in a sample, comprising: (a) obtaining peptide-exchanged MHCI complexes comprising a peptide of interest; (b) performing size exclusion chromatography or capillary electrophoresis, such as capillary zone electrophoresis, on the peptide exchanged MHCI complexes; and (c) following the chromatography or capillary electrophoresis of (b), performing native mass spectrometry (MS) on the MHCI complexes to identify MHCI complexes that comprise peptides of interest. In some embodiments, SEC is used in step (b). In some embodiments, CE is used in step (b). In some cases, CZE is used in step (b). In some embodiments, no further (i.e. no second dimension) chromatography or other separation process is performed between the SEC, CE, or CZE and the MS analysis. Thus, in some embodiments, the native MS directly follows the SEC, CE or CZE without further wash, buffer exchange, or chromatography steps.

In some embodiments of the methods above, part (a) further comprises performing peptide-exchange on the MHCI molecule. For example, peptide exchange may be facilitated by using a first peptide that can be cleaved or modified so as to reduce its affinity for the MHCI binding pocket, rendering it susceptible to competition from a peptide for analysis. Peptide exchange can be done, for example, using a first, exchangeable peptide that can be modified or cleaved upon exposure to UV light, particular chemicals or enzymes, for example, so that its affinity for the MHCI binding pocket is reduced. (See, e.g. Rodenko, B. et al., “Generation of peptide-MHC class I complexes through UV-mediated ligand exchange,” Nature Protocols, 1: 1120-32 (2006)).

The methods herein are compatible with a variety of types of samples and experimental contexts. For example, in some methods, the sample may be a biological fluid sample from a subject. In some methods, the sample may be a whole blood or a plasma sample. In some methods, the sample comprises T-cells, such as CD8+ and/or CD4+ T-cells. In some methods, the sample comprises peripheral blood mononuclear cells (PBMCs). In other methods, the sample is not derived from a subject. For example, in some methods, the sample comprises one or more synthetically produced peptides, for instance, to test their binding to particular types of MHCI complexes. In some methods, an array or library of different peptide exchanged MHCI complexes may be provided for analysis, optionally with many different peptides of interest mixed with different MHCI complexes comprised from different alpha and beta chains. In some aspects, these different peptide-MHCI complexes may be arranged in an array comprising many sample wells, for example, with each well comprising a unique peptide of interest and/or MHCI complex. In this way, the methods herein could be used, for example, to determine which peptides of interest will noncovalently bind to particular MHCI complexes.

Some embodiments herein may include monitoring MHCI complexes to determine whether certain peptides of interest are bound by T-cells. Thus, some embodiments may comprise (a) obtaining peptide-exchanged MHCI complexes comprising a peptide of interest; (b) contacting the peptide-exchanged MHCI complexes with a sample comprising T-cells (e.g. a biological fluid sample such as whole blood or plasma); (c) separating T-cell bound MHCI complexes from unbound MHCI complexes; (d) performing size exclusion chromatography or capillary electrophoresis (e.g. CZE) on the peptide exchanged MHCI complexes; and (e) following the chromatography or capillary electrophoresis of (d), performing native mass spectrometry (MS) on the MHCI complexes to identify MHCI complexes comprising peptides recognized by T-cells from the sample. For example, in some cases the T-cell bound MHCI complexes vs. the unbound complexes are separated by fluorescence-assisted cell sorting (FACS) or flow cytometry. In some cases, separating T-cell bound MHCI complexes from non-T-cell bound complexes is performed before the chromatography or capillary electrophoresis. This may allow for determination of which peptide-MHCI complexes the T-cells recognize. In some embodiments, SEC is used in step (b). In other embodiments, CE is used in step (b). In some cases, CZE is used in step (b). In some of the above embodiments, no further (i.e. no second dimension) chromatography or other separation process is performed between the SEC, CE, or CZE and the MS analysis. Thus, in some embodiments, the native MS directly follows the SEC, CE or CZE without further wash, buffer exchange, or chromatography steps. In some embodiments, part (a) of the method further comprises performing peptide-exchange on the MHCI complexes. For example, peptide exchange may be facilitated by using a first peptide that can be cleaved or modified so as to reduce its affinity for the MHCI binding pocket, rendering it susceptible to competition from a peptide for analysis.

In some embodiments, size exclusion chromatography (SEC) is used to separate molecules in the sample prior to MS. In other embodiments, capillary electrophoresis is used to separate molecules in the sample prior to MS. In some embodiments of the methods above, the capillary electrophoresis (CE) is capillary zone electrophoresis (CZE). Other types of CE are also available, including capillary gel electrophoresis (CGE), micellar electrokinetic capillary chromatography (MEKC), capillary electrochromatography (CEC), capillary isoelectric focusing (CIEF), and capillary isoelectrophoresis (CITP), among others.

The native MS analysis may have a variety of options. In some embodiments, the MS instrument does not comprise a quadrupole. In some embodiments, the MS instrument comprises at least one quadrupole. In some embodiments, the MS instrument comprises at least two quadrupole analyzers. In some embodiments, the MS instrument comprises at least three quadrupole analyzers. In some MS's, the detector is an ion trap, quadrupole, orbitrap, or TOF. In some embodiments, the MS instrument or method is multiple reaction monitoring (MRM), single ion monitoring (SIM), triple stage quadrupole (TSQ), quadrupole/time of flight (QTOF), quadrupole linear ion trap (QTRAP), hybrid ion trap/FTMS, time of flight/time of flight (TOF/TOF), Orbitrap instruments, ion trap instruments, parallel reaction monitoring (PRM), data dependent acquisition (DDA), data independent acquisition (DIA), multi-stage fragmentation or tandem in time MS/MS.

In some embodiments, SEC-native MS or CE-native MS (such as CZE-MS) is performed in a buffer that does not significantly ionize during the MS. In some embodiments, the native MS is performed in a buffer such as ammonium acetate or ammonium formate. In some embodiments, native MS is not performed with ionizing buffers such as TRIS or PBS. When SEC or CE directly precedes native MS, therefore, in some embodiments the SEC or CE is performed in a buffer that does not significantly ionize during MS, or is performed in a buffer such as ammonium acetate or ammonium formate.

In some embodiments, native MS is performed via electrospray ionization into an Orbitrap™ MS instrument (e.g. Thermo Exactive™ Plus EMR, ThermoFisher Scientific). In some embodiments, particular MS parameters are optimized to allow native MS on MHCI peptide-exchanged complexes. For example, exemplary parameters are provided in Table 1 provided following the Examples section (see right two columns) and are compared to parameters used for other SEC-MS protein separations (left two columns). In some embodiments, the aux gas flow rate is set to a value of 0-4, such as a value of 0-3, such as 0-2, or 0. In some embodiments, the native MS is performed with the aux gas flow rate at 0. In some embodiments, the in source CID parameter is also set to a value of zero when performing SEC-MS. In some embodiments, the sheath gas flow rate is less than 15, such as 1-5 or 2-4. In some embodiments, the trapping gas pressure is set to 2-3.

In some embodiments, the CE-native MS or SEC-native MS methods herein allow for confirmation that a peptide of interest is actually bound noncovalently in the binding pocket of a MHCI molecule, as the bound complex remains associated during the MS analysis. Thus, for example, the methods herein allow not only for assessment of whether peptide exchange has occurred, but also for the determination and quantitation of the percentage peptide exchange for different peptides of interest. In some embodiments, the native MS analysis also allows for at least partial sequencing of a peptide of interest. In contrast, in other LC-MS methods that do not use native MS, MHCI complexes fall apart during MS meaning that they cannot be used to confirm that an associated peptide is actually bound noncovalently in the MHCI binding pocket.

In some embodiments, the methods may be performed more rapidly than 2D-LC-MS analysis methods. They also allow for relatively low volumes for injection into MS equipment. In certain SEC-native MS methods herein, volumes on the order of 2-10 μL may be injected, such as 3-6 μL or 4-5 μL. In certain EC-native MS methods herein, volumes of, on the order of 2-100 nL may be injected, such as 2-50 nL, 2-10 nL, 3-10 nL, or 3-5 nL. In some such cases using CE or CZE, peptide concentrations on the order of 100 μg/mL may be detected, such as 100 μg/mL or lower, 50-500 μg/mL, 50-200 μg/mL, 100-200 μg/mL, or 50-100 μg/mL.

Capillary electrophoresis methods may have additional benefits in some embodiments. For instance, in some embodiments, when CZE is used, the electrophoresis separation may be performed in 1-10 minutes, such as in 1-5 minutes or 2-5 minutes. In some embodiments, CE methods herein may allow for detection of particular peptides of interest in a sample following peptide exchange at lower concentrations than LC-MS methods. In some embodiments, a reduced sample volume may be used in CE methods, such as those in which the capillary is provided on a chip or cartridge. Thus, for example, using some CE platforms, volumes of, for example, For example, in some embodiments, an SEC-MS method did not allow detection of peptides below about 100 μg/mL concentration, whereas a CZE method was able to detect peptides of below about 100 μg/mL concentration. In some embodiments, particular peptides of interest bound within MHCI complexes may be detected by the CE-MS methods herein at concentrations of, for example, 100 μg/mL or lower, 50-500 μg/mL, 50-200 μg/mL, 100-200 μg/mL, or 50-100 μg/mL. This potentially higher resolution may be helpful in picking out particular MHCI-antigen peptide complexes. For example, certain cancers may be characterized by mutations in several possible genes, potentially giving rise to a number of neoantigens for analysis (e.g., up to 50 or up to 100 possible neoantigens). When these are combined with the large number of possible human MHCI complexes, for example, this can result in possibly thousands of potential neoantigen-MHCI combinations, each at relatively low concentrations following peptide exchange.

### Kits

In embodiments, a kit containing a peptide comprising a non-natural UV-cleavable amino acid, MHCI alpha chain monomers, and MHCI beta chain monomers. In some embodiments, the kit contains MHCI alpha chain monomers that are denatured. In some embodiments, the kit contains MHCI beta chain monomers that are denatured. In some embodiments, both the MHCI alpha chain and MHCI beta chain monomers are denatured.

In embodiments, the kit contains tagged MHCI alpha chain and tagged MHCI beta chain. In some embodiments, the MHCI alpha chain contains a tag. In some embodiments, the MHCI beta chain contains a tag. In some embodiments, both the MHCI alpha chain and MHCI beta chain are tagged. In some embodiments, the tag is streptavidin.

In embodiments, the kit contains an anti-HLA antibody. In embodiments, the kit contains an anti-B2M antibody. In embodiments, the kit contains an anti-HLA antibody and an anti-B2M antibody.

In embodiments, the kit contains a peptide containing a non-natural UV-cleavable amino acid, wherein the peptide has an amino acid sequence of any one of SEQ ID NO.: 1 to SEQ ID NO.: 34. In some embodiments, the peptide contains the sequence SEQ ID NO.: 1. In some embodiments, the peptide contains the sequence SEQ ID NO.: 2. In some embodiments, the peptide contains the sequence SEQ ID NO.: 3. In some embodiments, the peptide contains the sequence SEQ ID NO.: 4. In some embodiments, the peptide contains the sequence SEQ ID NO.: 5. In some embodiments, the peptide contains the sequence SEQ ID NO.: 6. In some embodiments, the peptide contains the sequence SEQ ID NO.: 7. In some embodiments, the peptide contains the sequence SEQ ID NO.: 8. In some embodiments, the peptide contains the sequence SEQ ID NO.: 9. In some embodiments, the peptide contains the sequence SEQ ID NO.: 10. In some embodiments, the peptide contains the sequence SEQ ID NO.: 11. In some embodiments, the peptide contains the sequence SEQ ID NO.: 12. In some embodiments, the peptide contains the sequence SEQ ID NO.: 13. In some embodiments, the peptide contains the sequence SEQ ID NO.: 14. In some embodiments, the peptide contains the sequence SEQ ID NO.: 15. In some embodiments, the peptide contains the sequence SEQ ID NO.: 16. In some embodiments, the peptide contains the sequence SEQ ID NO.: 17. In some embodiments, the peptide contains the sequence SEQ ID NO.: 18. In some embodiments, the peptide contains the sequence SEQ ID NO.: 19. In some embodiments, the peptide contains the sequence SEQ ID NO.: 20. In some embodiments, the peptide contains the sequence SEQ ID NO.: 21. In some embodiments, the peptide contains the sequence SEQ ID NO.: 22. In some embodiments, the peptide contains the sequence SEQ ID NO.: 23. In some embodiments, the peptide contains the sequence SEQ ID NO.: 24. In some embodiments, the peptide contains the sequence SEQ ID NO.: 25. In some embodiments, the peptide contains the sequence SEQ ID NO.: 26. In some embodiments, the peptide contains the sequence SEQ ID NO.: 27. In some embodiments, the peptide contains the sequence SEQ ID NO.: 28. V In some embodiments, the peptide contains the sequence SEQ ID NO.: 29. V In some embodiments, the peptide contains the sequence SEQ ID NO.: 30. In some embodiments, the peptide contains the sequence SEQ ID NO.: 31. In some embodiments, the peptide contains the sequence SEQ ID NO.: 32. In some embodiments, the peptide contains the sequence SEQ ID NO.: 33. In some embodiments, the peptide contains the sequence SEQ ID NO.: 34.

The disclosure herein also encompasses kits for or reagent compositions for conducting the SEC-native MS and CE-native MS or CZE-native MS methods described above. In some embodiments, such kits may include buffers for performing (a) SEC, EC or CZE separation, (b) peptide exchange, and (c) any other applicable wash or buffer exchange steps, or combinations of any of these types of buffers. In some embodiments, the SEC, EC, or CZE is performed in a buffer that does not significantly ionize during MS. In some embodiments, the SEC, EC, or CZE is performed in an ammonium acetate or ammonium formate buffer. In some embodiments, the buffer for SEC, EC, or CZE does not comprise Tris or PBS.

Kits or reagent compositions herein may also include reagents for performing peptide exchange, such as exchangeable peptides and any necessary reagents for modifying the binding affinity of such peptides such as appropriate chemicals or enzymes. Kits or reagent compositions herein may also include instructions for performing methods herein or portions of such methods. Kits or reagent compositions herein may also include arrays or libraries of MHCI complexes on which peptide exchange may be performed, such as an array or library of different human MHCI complexes. Kits or reagent compositions herein may also include a library of putative neoantigen peptides, for example, based on a pathogenic disease, type of tumor, or the like.

### Systems

In embodiments, a system containing: a peptide containing a non-natural UV-cleavable amino acid; a plurality of MHCI alpha chain monomers; a plurality of MHCI beta chain monomers; and a first reagent capable of allowing formation of a MHCI/ligand complex. In some embodiments, the system further contains a second reagent capable of binding a MHCI alpha chain monomer. In some embodiments, the second reagent contains an anti-HLA antibody. In some embodiments, the system contains a third reagent capable of binding a MHCI beta chain monomer. In some embodiments, the third reagent is an anti-B2M antibody

### EXAMPLES

### Example 1: High-Throughput MHCI Refolding

MHCI alpha chain monomers are either purified under denaturing conditions or denatured using standard denaturing reagents, including, but not limited to, 6 M guanidine-HCl, 6 M guanidine isothiocyanate, or 8 M urea.

Denatured alpha chain, B2M, and peptide ligands are added together for refolding and soluble MHCI/peptide complex formation. An example refolding protocol follows: B2M (10 mg/L), HLA (30 mg/L) and peptide (10 μM) are added to the refold buffer (100 mM Tris pH 8.0, 400 mM L-arginine and 2 mM EDTA) with 0.5 mM and 4 mM oxidized and reduced glutathione, respectively. These reagents are added to a final volume of 100 μL in a 96 well plate and after incubation for 1-10 days at 4° C., were tested for complex formation.

### Example 2: High-Throughput ELISA to Identify Peptides for MHCI Refolding

Conditional peptide ligands containing a non-natural UV cleavable amino acid were identified for 38 different MHCI complexes. The 38 different MHCI complexes consisted of a unique HLA allele (alpha chain), B2M, and unique peptide containing a non-natural UV cleavable amino acid.

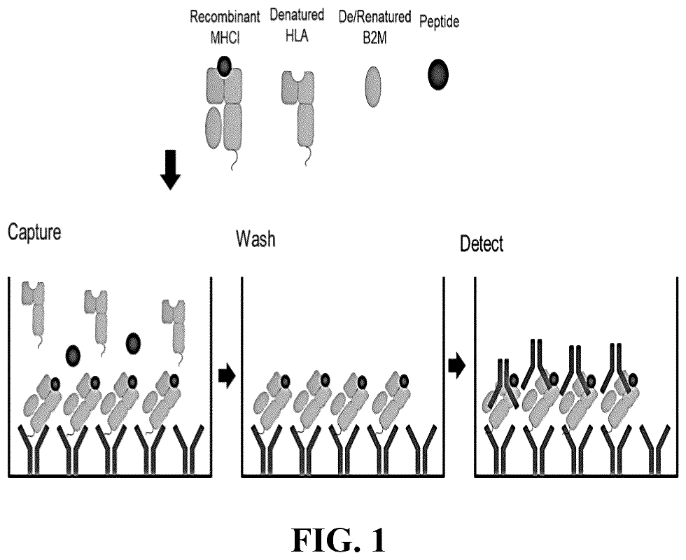

High-through put enzyme-linked immunosorbent assay (ELISA) to identify peptides that allow for proper MHCI refolding: One method for identifying epitope binders is to evaluate whether an MHCI of a given allele can form a stable refolded complex in the presence of the epitope. This process involves mixing denatured HLA and B2M at dilute concentrations with the epitope of interest. After a 2-5 day of refold, the diluted refolded MHCI reagent must be concentrated, purified and characterized, all of which are not amenable to high throughput. Here we developed an ELISA assay that can detect properly refolded MHCI at nM concentrations in the absence of purification. The ELISA assay involves signal amplification with biotinylated detection antibody and streptavidin. An example ELISA assay is shown in FIG. 1.

A 384 well Maxisorp plate (Thermo, Nunc #464718) was coated with 25 μL/well of anti-HLA mouse IgG1 monoclonal antibody (ABC W6/32, Cat #NB100-64775, Novus Biological) at 8 μg/mL in 0.05 M sodium carbonate, pH 9.6 and incubated overnight at 4° C. After washing the plate three times with wash buffer (PBS buffer with 0.05% Tween 20), 80 μL/well of block buffer (PBS, 0.5% BSA, 15 PPM Proclin) was added and incubated at room temperature (RT) for 1 hour. The plate was then washed three times with wash buffer and 25 μL of diluted samples containing MHCI monomers with peptides of interest were added into the appropriate wells. The plate was incubated at RT for 2 hours and the unbound components were removed by washing the plate six times with wash buffer. The bound MHCI monomer-peptide complex were then detected by adding 25 μL/well of the biotinylated anti-human 132-microglobulin (B2M) mouse IgG2a monoclonal antibody (Cat #316302, Biolegend) in assay buffer (PBS 0.5% BSA, 0.05% Tween 20, 15 PPM Proclin, pH 7.4) at 100 ng/mL and incubated at RT for 1 hour. After six washes, 25 μL/well of horseradish peroxidase conjugated streptavidin (HRP-SA) in assay buffer was added and incubate at RT for 30 mins. After removing the HRP-SA with 3 washes, the enzymatic reaction was developed with the peroxidase substrate tetramethylbenzidine (TMB, Moss Inc. cat #1000) and incubated at RT for 15 mins. The reaction was stopped with 25 μL/well of 1M phosphoric acid and the absorbance was measured at 450 nm using reference 620 nM on a Multiscan spectrophotometer (ThermoFisher).

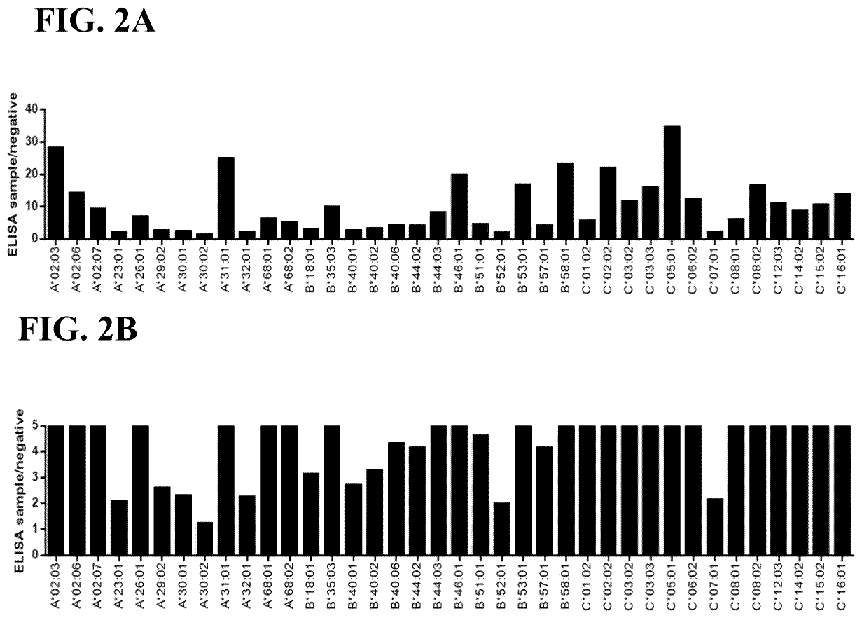

Refolded MHCI heterodimer complexes without peptide ligand or irrelevant peptide were used as the negative control for each allele. The sample signal was normalized as shown in the following ratio: OD450/620[sample]/OD450/620[negative control]. Results are provided in FIGS. 2-5, and 7.

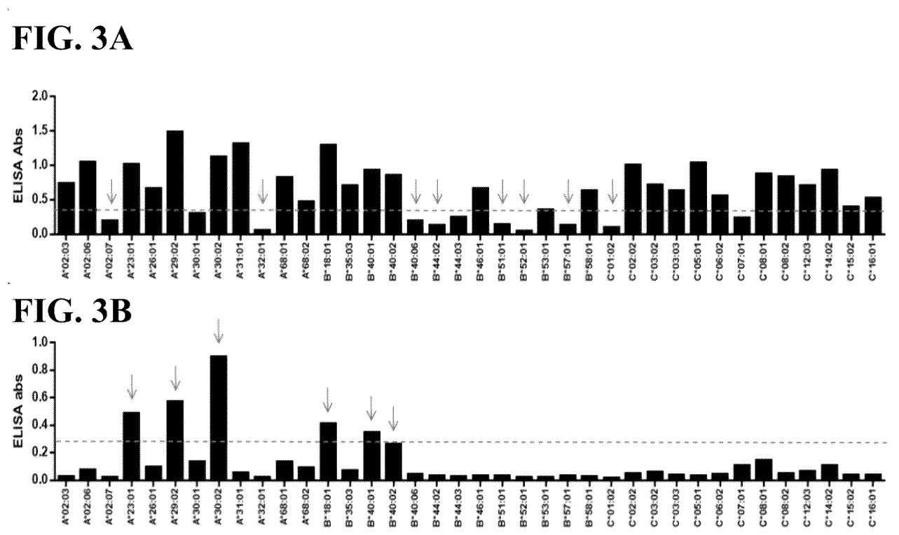

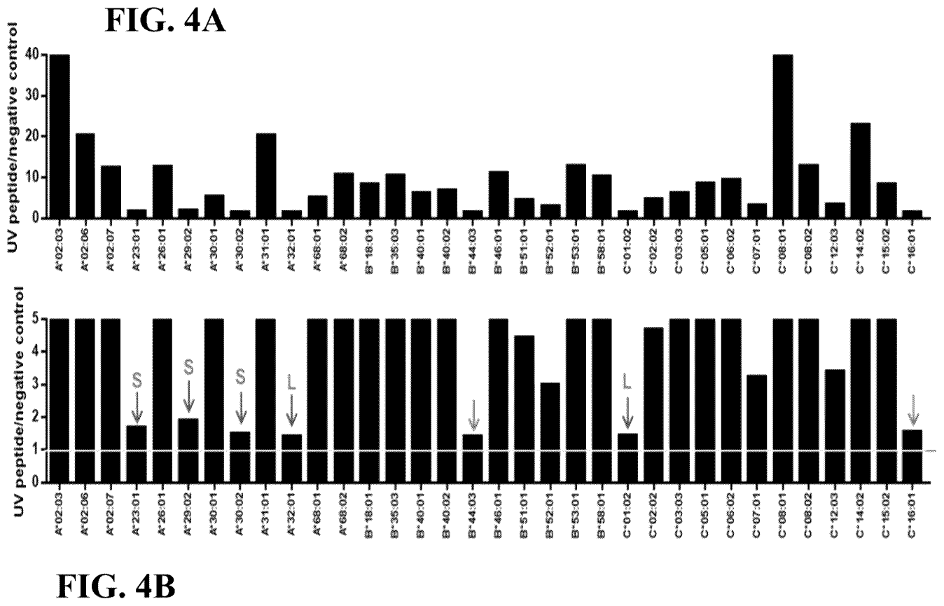

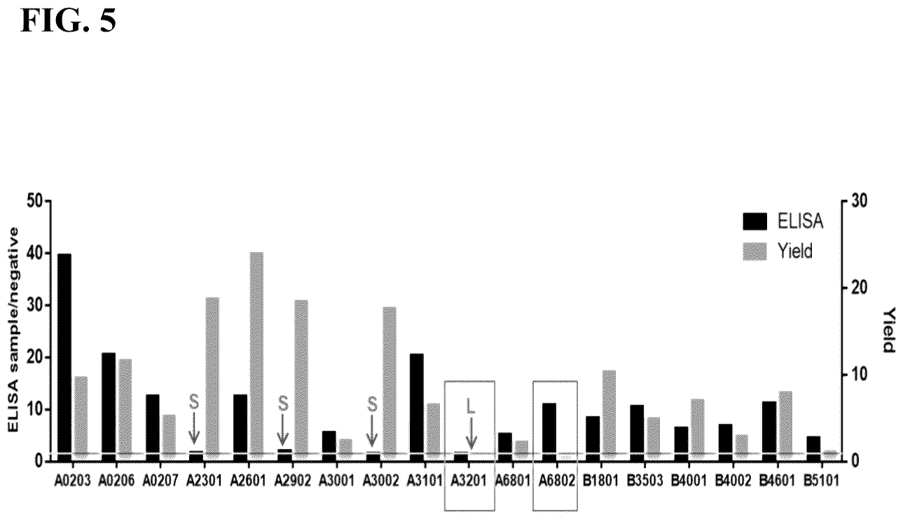

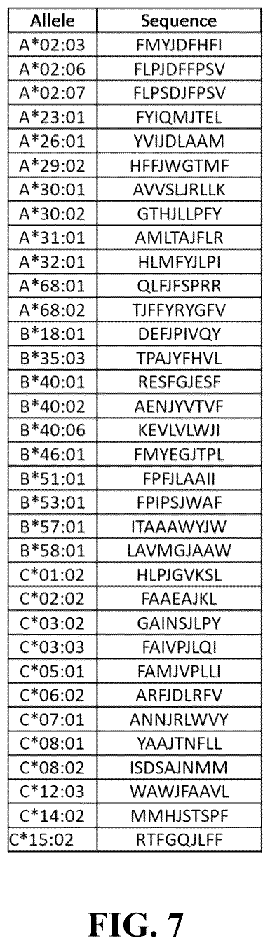

FIG. 2 Panels A and B are bar graph representations of the normalized (vs. no peptide present, sample signal/negative control signal) ELISA signals of captured MHCI/B2M/peptide complexes (via secondary antibody reporter) averaged across 38 different HLA, HLB, and HLC binders. FIG. 3 Panels A and B are bar graph representations of averaged ELISA signal over MHCI/B2M/peptide binders, showing specific MHCI alleles that are low capture antibody binders (Panel A) and specific MHCI/B2M complexes that are stable without the presence of peptide (Panel B). FIG. 4 Panels A and B are bar graph representations of averaged ELISA signal over MHCI/B2A/UV peptide binders, in the presence of UV peptide (a peptide containing a UV-cleavable amino acid). FIG. 5 shows a bar graph comparing normalized ELISA results and yield from a scaled up refolding purification of 18 different HLA/HLB alleles. FIG. 7 provides a list of UV peptides that bind each allele, selected based on the peptide that performed the best in the ELISA assay.

### Example 3: High-Throughput Time-Resolved Fluorescence Resonance Energy Transfer Assay to Identify Peptide Binders

High-through put time-resolved-fluorescence energy transfer (TR-FRET) assay to identify MHCI peptide binders after peptide exchange: Another method for identifying epitope binders is to generate MHCI complexes with conditional ligands that can be dissociated from the complex when provided a given trigger and allow a peptide binder to exchange into the MHCI groove. One way to achieve this is using a peptide containing a UV-cleavable non-natural amino acid such that, when exposed to UV, the peptide is cleaved and is no longer a binder. Under these conditions, if there is a peptide binder in solution it will bind to and stabilize the complex. In contrast, in most cases, if there is no peptide binder in solution, the HLA and B2M proteins will disassociate and the MHCI complex will fall apart.

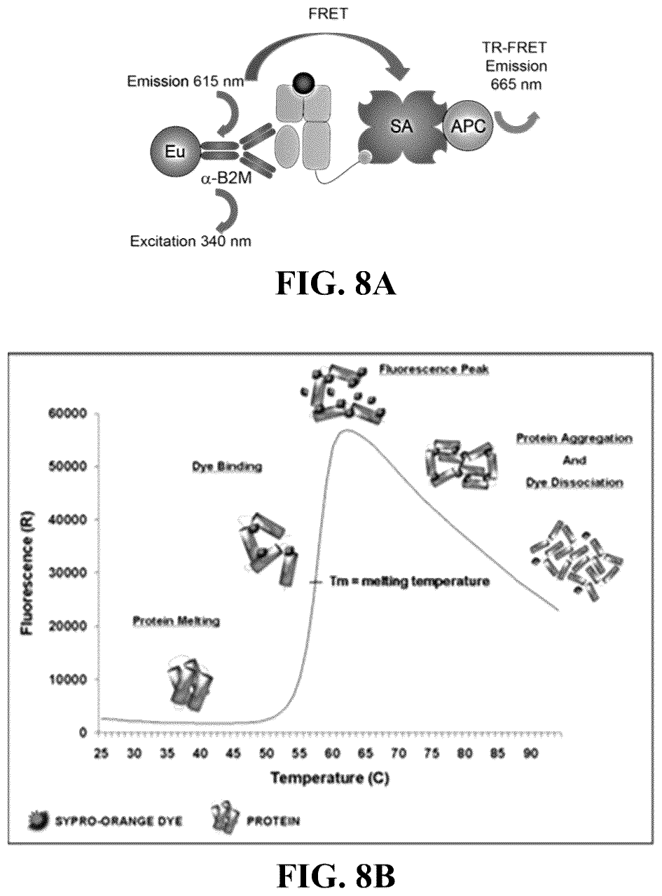

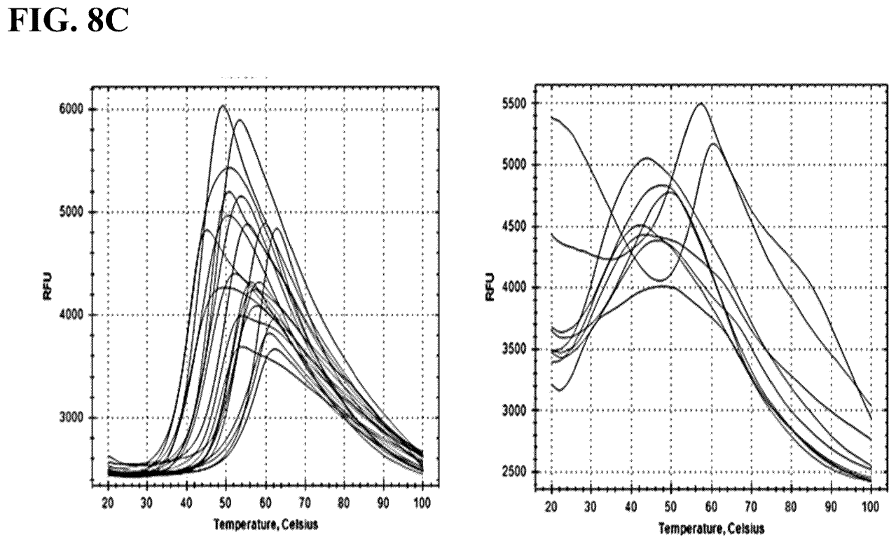

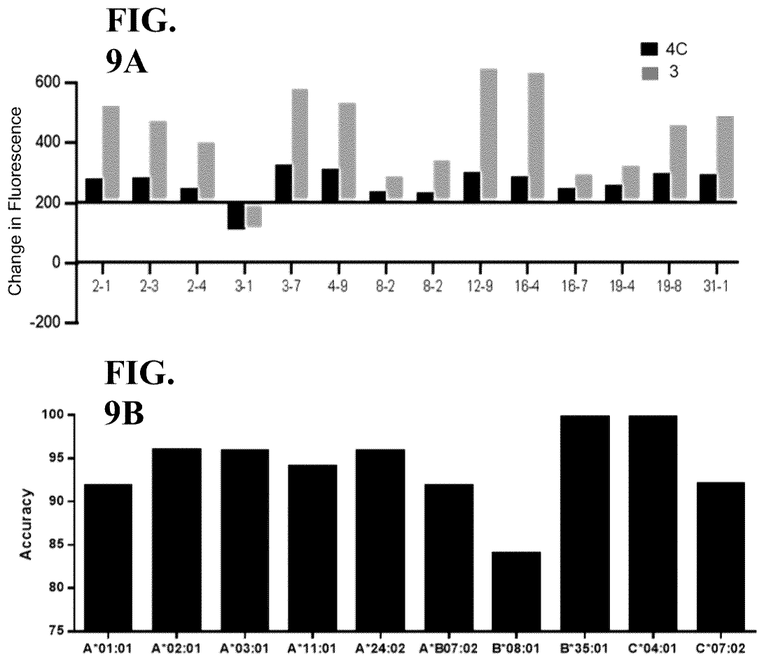

To use this system to determine MHCI binders, a TR-FRET assay was developed that provides a signal only when the B2M and HLA complex are in close proximity. This assay uses an antibody against B2M that contains a TR-FRET donor (anti-B2M-donor) and streptavidin, which binds to the biotinylated HLA component of the complex, labeled with a TR-FRET acceptor (Streptavidin-Allophycocyanin (SA)-acceptor). If B2M and HLA are complexed together than anti-B2M donor and SA-acceptor will be close in solution resulting in a TR-FRET signal. In contrast, if the complex falls apart these reagents will be evenly distributed and there will be no TR-FRET signal. A schematic of the TR-FRET assay workflow is provided in the panels of FIG. 8. This assay was applied to peptide exchange after exposure to UV light for the MHCI molecules generated with the UV cleavable conditional ligands and successfully used to identify MHCI peptide binders.

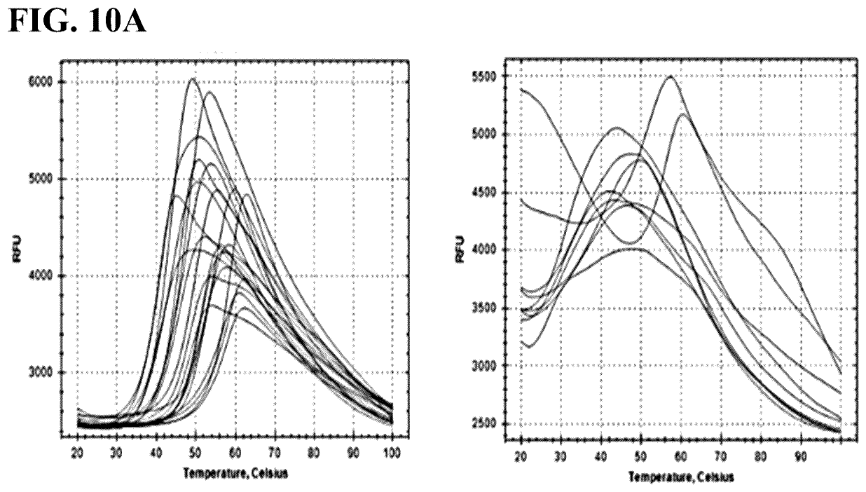

A 384 well source plate (Echo Qualified 384-Well Polypropylene 2.0 Plus Microplate, Labcyte PPL-0200) containing UV-exchanged MHCI/peptide complex was incubated at 37° C. overnight. The plate was equilibrated at RT for 1 hour followed by centrifugation. Each well of the source plate was dispensed four times at various volumes (160 nL, 80 nL, 40 nL and 20 nL) with an automated acoustic dispenser (Echo 550, Labcyte) into the back filled wells (with assay buffer) of the destination plate (MAKO 1536 well white solid bottom, Aurora Microplates, MT, USA) for a total volume of 2 μL/well with final sample concentrations at 10, 5, 2.5 and 1.25 nM. A 2 μL mixture of donor (1.2 μg/mL of Europium (Eu) LANCE-W1024-ITC labeled B2M) and acceptor (4.8 μg/mL of Sure Light Allophycocyanin conjugated Streptavidin (Perkin Elmer) in assay buffer was dispense into each well of the destination plate. The destination plate was then centrifuged and incubated at room temperature for one hour, the TR-FRET signals were recorded using PHERAstar FSX plate reader (BMG Labtech, NC, USA) equipped with HTRF Module (Eu excitation 337 nm, Eu emission 615 nm; APC emission 660/20 nm). The TR-FRET raw signals were expressed as ratios of relative fluorescent unit (RFU ratio=(RFU[660 nm]/RFU[615 nm×10⁴]). The detection window was calculated by subtracting the background signal from the assay mix in the absence of MHCI/peptide complex. Results for a range of peptide binders are provided in the bar graphs in Panel A of FIG. 9. A bar graph of the relative accuracy (85%-100%) of the TR-FRET assay vs. MHC allele is provided in Panel B of FIG. 9.

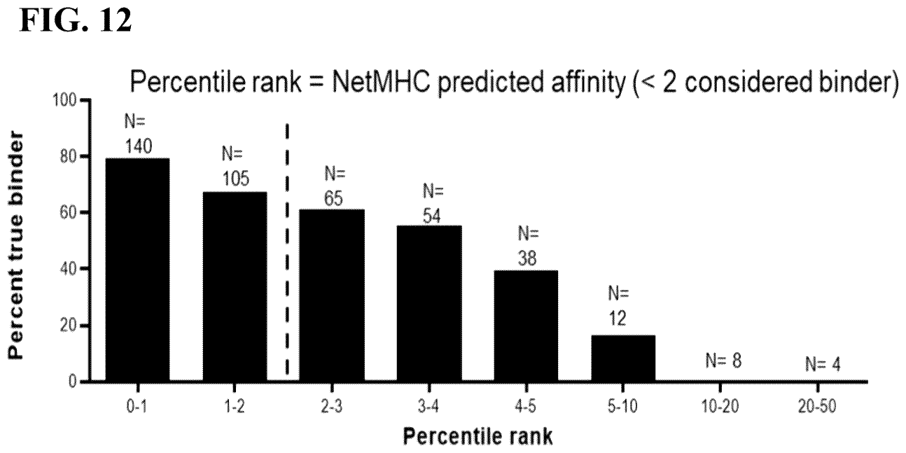

A peptide was determined to be a true binder based on a comparison to its predicted binding affinity (calculated using a binding prediction algorithm based on Andreatta M, and Nielsen, M. Gapped sequence alignment using artificial neural networks: application to the MHC class I system. Bioinformatics (2016) Feb. 15; 32(4):511-517. The peptide binders and corresponding MHCI alleles were identified using the TR-FRET and 2D LC/MS assays. The peptide sequences were submitted to the prediction algorithm, and each sequence was assigned a percentile rank. A percentile rank of 2 or less it is considered a binder. FIG. 12 shows the comparison of % true binder calculated from TR-FRET and LC-MS and the algorithm generated percentile rank. Only peptides to the left of the dashed line are predicted to be binders by the algorithm.

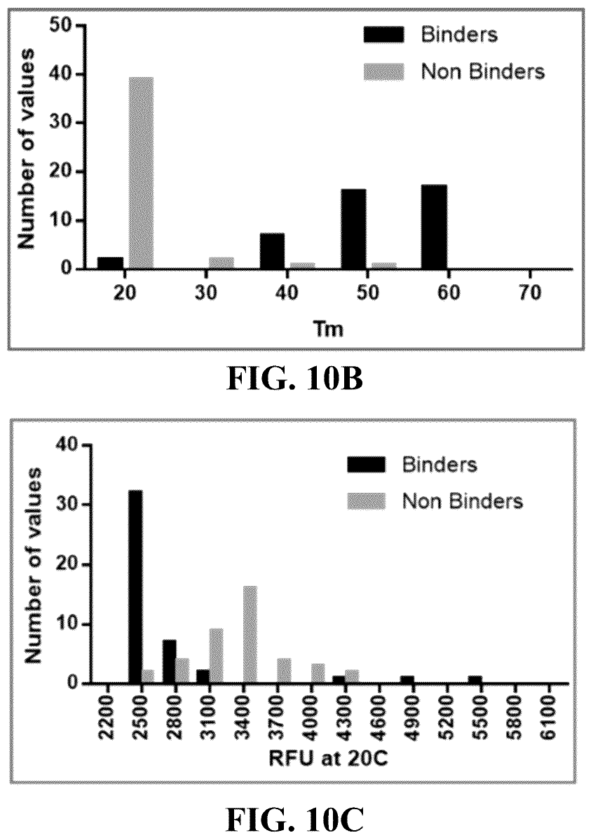

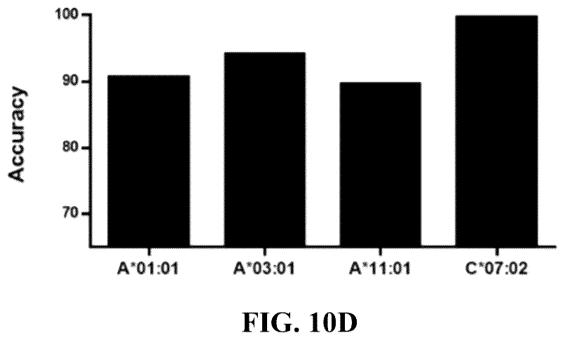

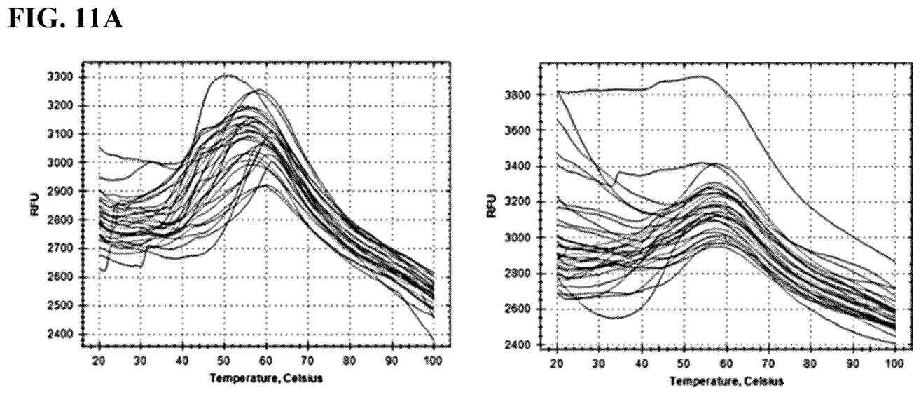

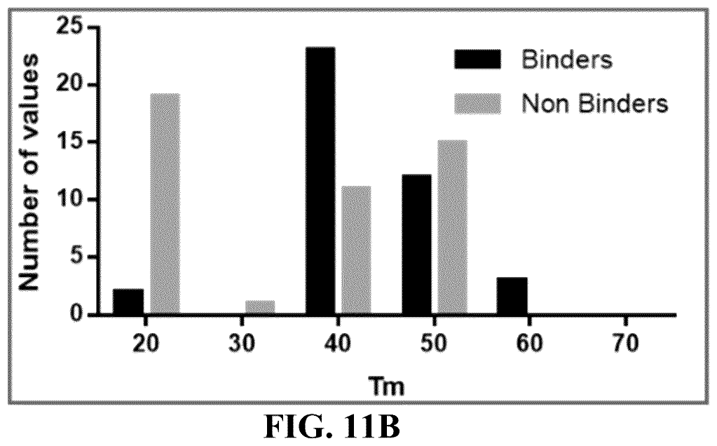

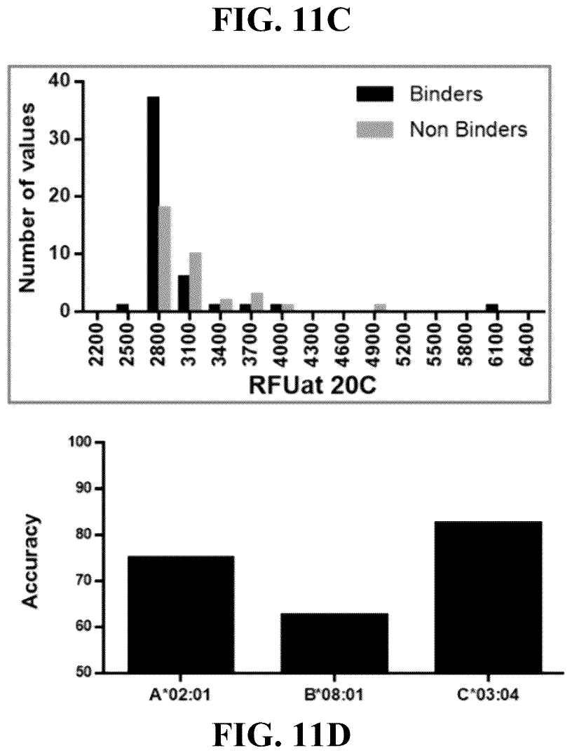

Two representative TR-FRET experiments are shown in FIG. 10 and FIG. 11. FIG. 10 shows comparative DSF spectra comparing peptide binders and non-binders of MHCI HLA*03:01 containing complexes. At low temperatures (20° C.), relative fluorescence (RFU) is low for complexes where there is a peptide bound, and high for complexes without peptide binding. The peptide binder DSF spectra show a similar range of temperatures for Tm, whereas the non-binders have lower Tm. Panel B is a bar graph of total number vs. Tm temperature for peptide binders (black) and non-binders (grey). Panel C is a bar graph of total number vs. RFU values for peptide binders (black) and non-binders (grey). FIG. 10 shows comparative DSF spectra comparing peptide binders and non-binders of MHCI HLA*08:01 containing complexes. At low temperatures (20° C.), relative fluorescence (RFU) is low for complexes where there is a peptide bound, and high for complexes without peptide binding. The peptide binder DSF spectra show a similar range of temperatures for Tm, whereas the non-binders have lower Tm. Panel B is a bar graph of total number vs. Tm temperature for peptide binders (black) and non-binders (grey). Panel C is a bar graph of total number vs. RFU values for peptide binders (black) and non-binders (grey). Panel D in FIG. 10 and FIG. 11 are a bar graph representation of the % accuracy of the assay for four different MHCI alleles (all >90%, FIG. 10, or 60-85%, FIG. 11).

### Example 4: LC-MS Assay to Identify Peptide Binders

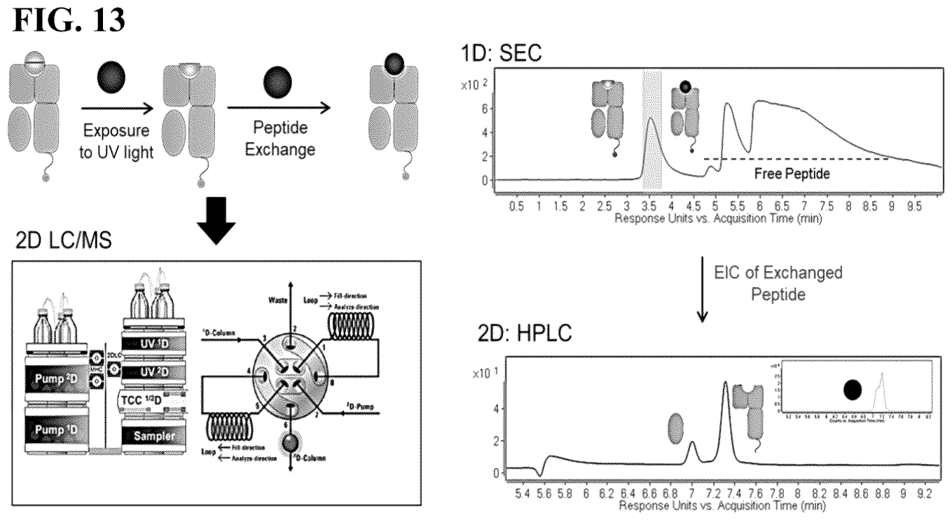

2D LC-MS assay to identify MHCI peptide binders: This assay was developed to identify peptide binders after a peptide exchange process for MHCI reagents. Mass spectrometry based analysis of the peptide exchange process depends on first separating out the MHCI complex from free peptide in solution prior to analysis. This would require complex up front purification and would not be amenable to high through put analysis. A 2D LC-MS analysis method was developed, where the sample is first run on an SEC column and then only the peak that corresponds to the MHCI complex is injected onto the second HPLC column for mass spectrometry analysis. This allows for complete analysis of the MHCI reagent in a single step. This process can be used to identify a single peptide binder at a time or binders within a larger pool of peptides. FIG. 13 provides a schematic of the peptide exchange and identification assay.

Briefly, between 2-3 μg of MHCI/peptide mixtures were injected on the instrument and sent to the first dimension column. The first dimension LC method employed an analytical size exclusion column (SEC) (Agilent AdvanceBio SEC 300 Å, 2.7 μm, 4.6×15 mm) to separate intact complex from excess peptide run at an isocratic flow of 0.7 mL/min in 25 mM Tris pH 8.0, 150 mM NaCl for 10 min with signals acquired at 280 nm. A sampling valve collected the entirety of the complex peak that eluted between 1.90-2.13 min in a volume of 160 μL and injected it onto the second dimension reversed-phase column (Agilent PLRP-S 1000 Å, 8 μm, 50×2.1 mm). The second dimension column was run with a gradient of 5-50% mobile phase B in 4.7 min at 0.55 mL/min with the column heated to 80° C. Mobile phase A was 0.05% TFA in water. Mobile phase B was 0.05% TFA in acetonitrile. The column eluent was sent to an Agilent 6224 TOF LC-MS for mass spectrometry data acquisition.

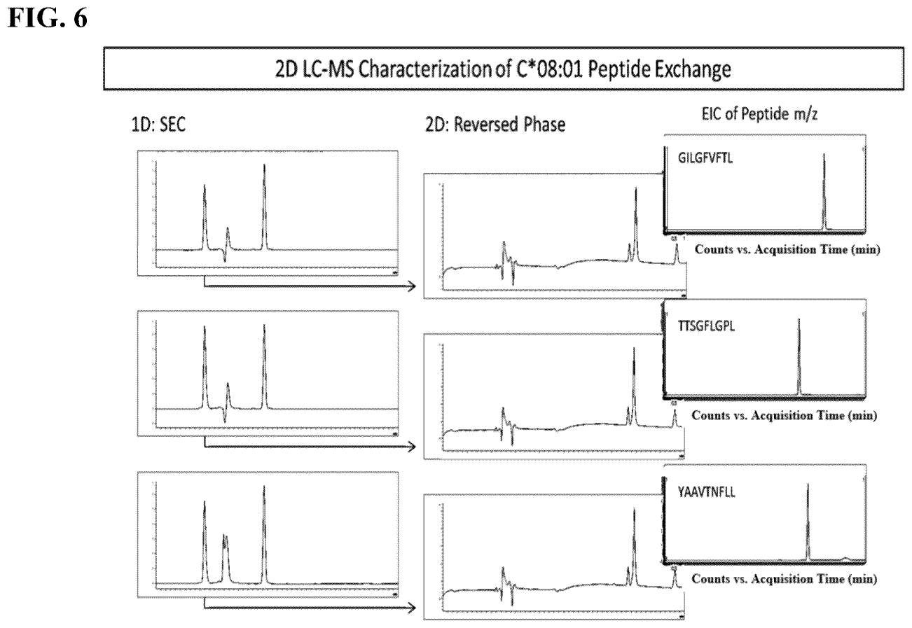

The MHCI/peptide complex peak area in the first dimension and mass spec detection of the peptide in the second dimension are used to determine successful peptide binding. Successful binding of a peptide into the complex after cleavage of the conditional ligand during the peptide exchange reaction stabilizes the complex and results in nearly complete recovery of the starting complex measured in the first dimension SEC analysis. The peptide that has exchanged into the complex can then be detected in the second dimension, where the complex is run under denaturing conditions with mass spectral analysis allowing for direct detection of the peptide of interest. Unsuccessful peptide exchange reactions result in destabilized complex after the cleavage of the conditional ligand when a peptide fails to bind to and stabilize the complex. This is measured as a reduction in A280 peak area of the complex on SEC and an absence of peptide in the second dimension (see far left set of chromatograms in FIG. 6 for examples of SEC chromatograms). In some cases, no reduction in peak area is observed; however the peptide is not detected by mass spectrometry. A small number of peptides are not captured by the second dimension chromatography column and method. In these cases, the peak area recovery is enough to determine successful exchange when positive and negative controls for peptide binding are also used.

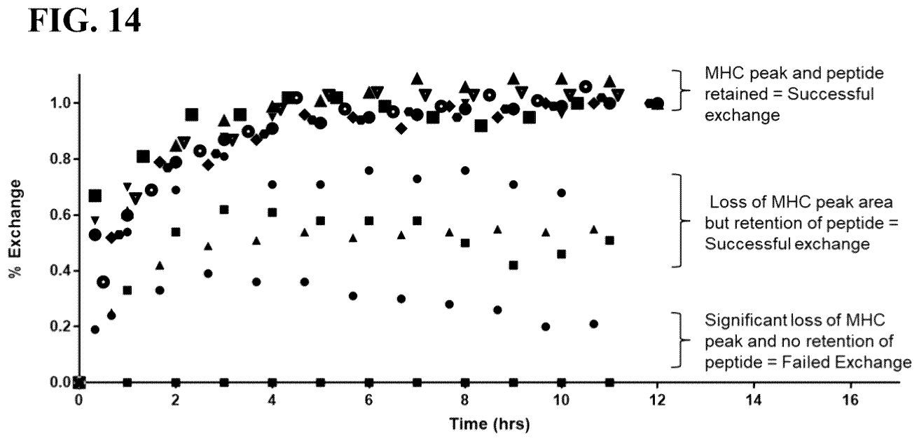

A representative set of chromatograms for a peptide exchange time course for HLC*08:01 is shown in FIG. 6. The first set of chromatograms are the result of running the complex mixture on a size-exclusion column after the refolding time course, with the peak containing the MHCI/peptide highlight in grey in each chromatogram (1D: SEC). The contents of this peak were collected, and injected onto a second HPLC equipped with reversed-phase column. The second set of chromatograms shows the contents of the complex peak, and the identity of the peak components were determined by mass spectrometry. The sequence of the peptide present in the complex was determined and the sequence is shown overlaid in the blow-up inserts of the corresponding extracted ion chromatogram (EIC) for the identified peptide peak from the reversed-phase chromatogram. FIG. 14 shows a representative validation panel of 10 peptides as exchangers or non-exchangers for each allele, shown as a plot of % exchange over time for a range of peptides with the MHCI/B2M/UV-peptide complex as measured by 2-D LC/MS.

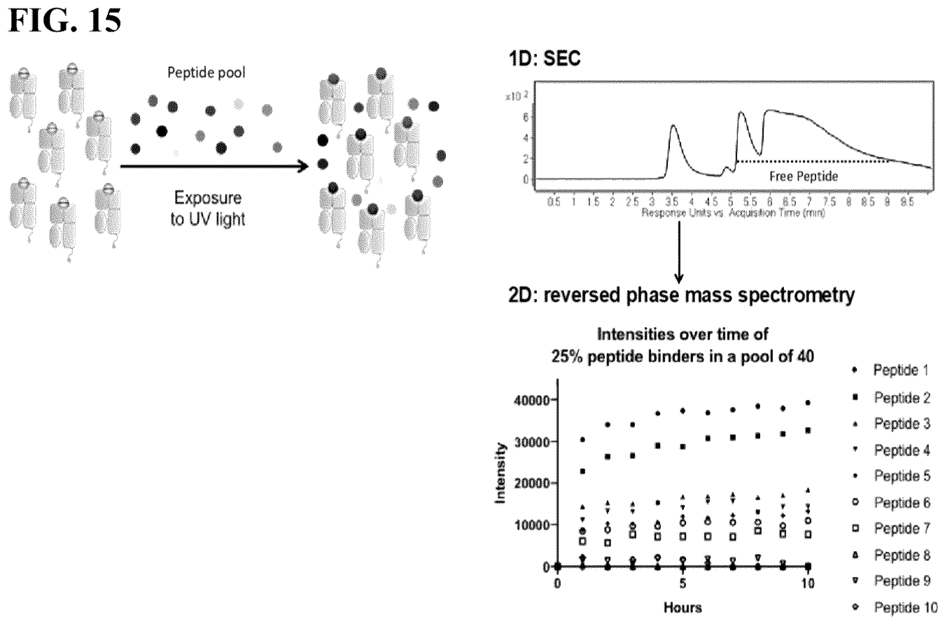

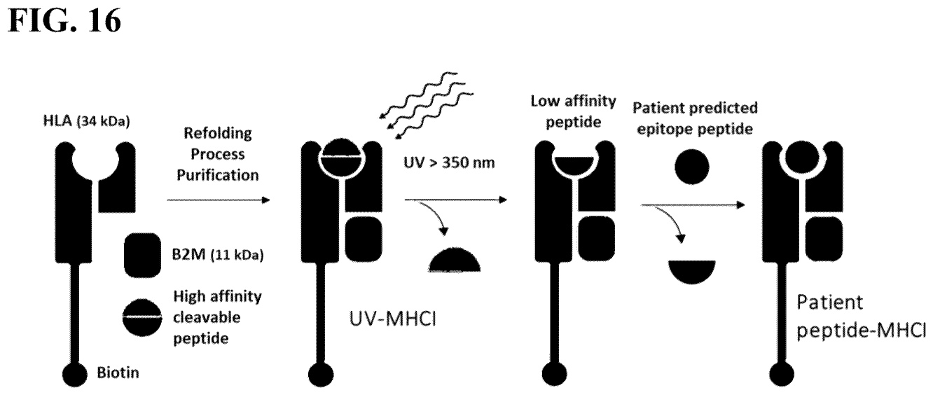

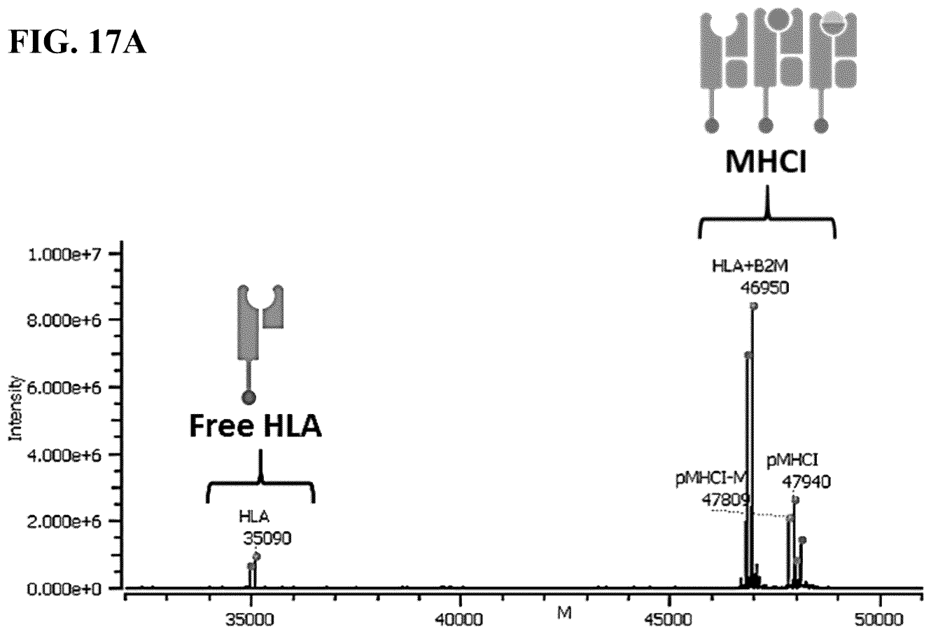

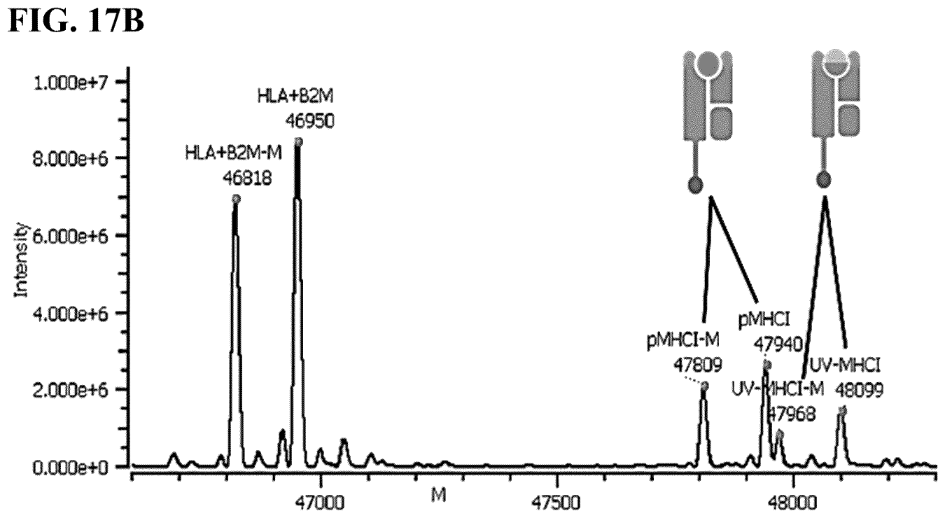

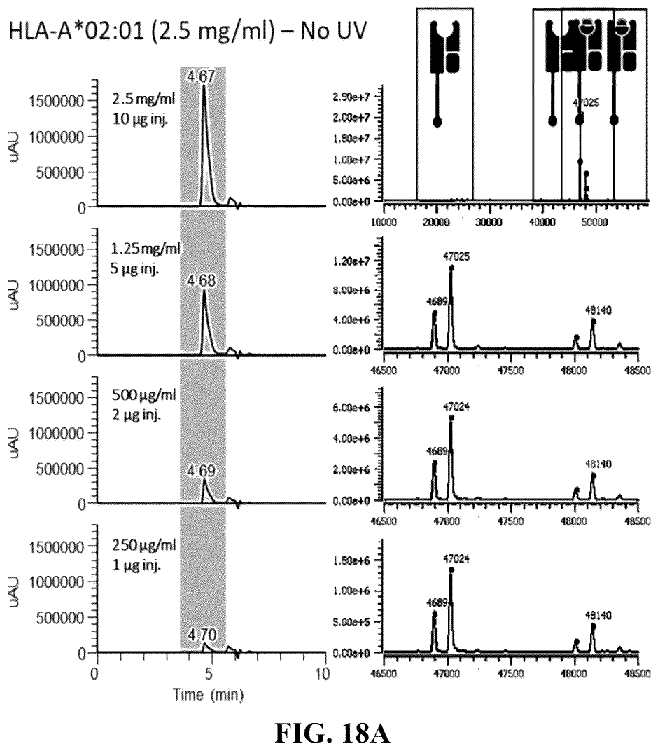

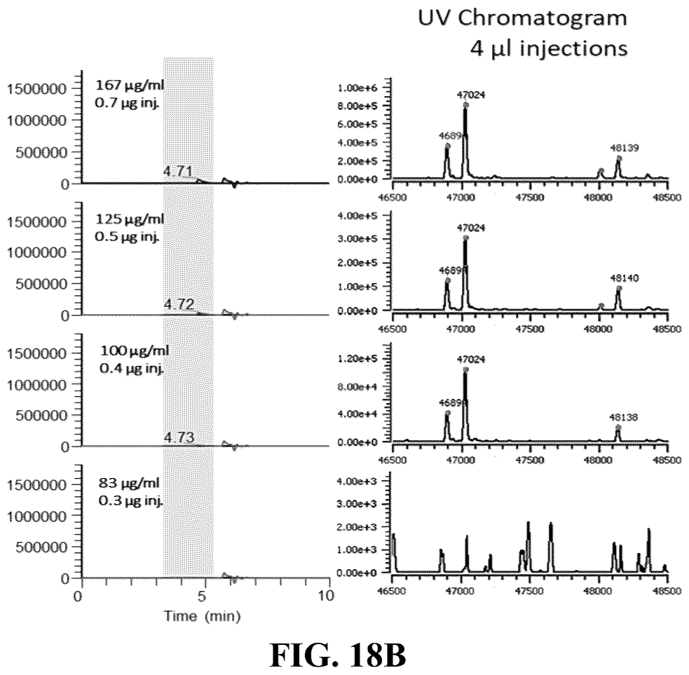

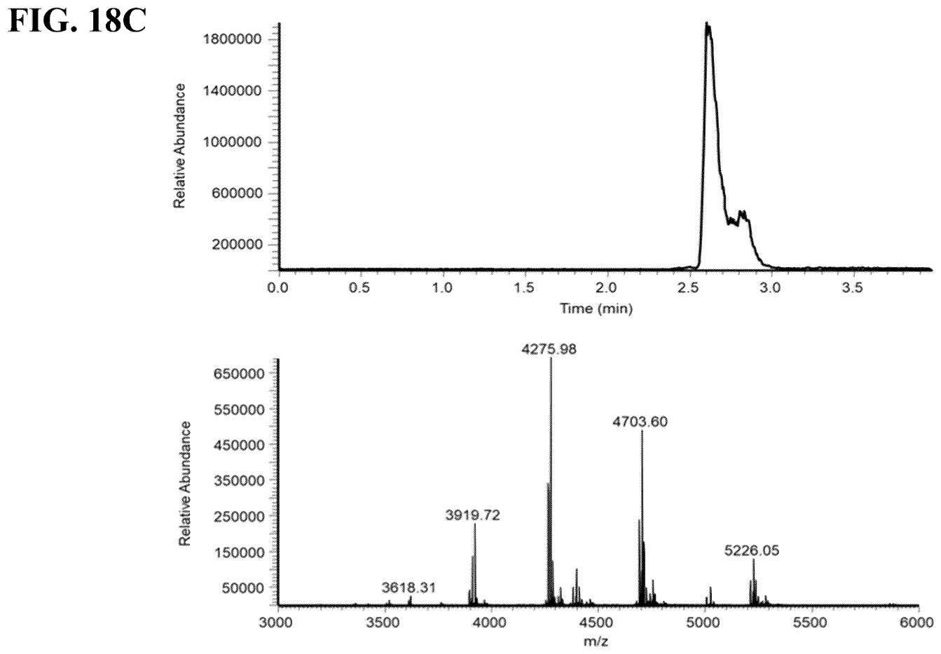

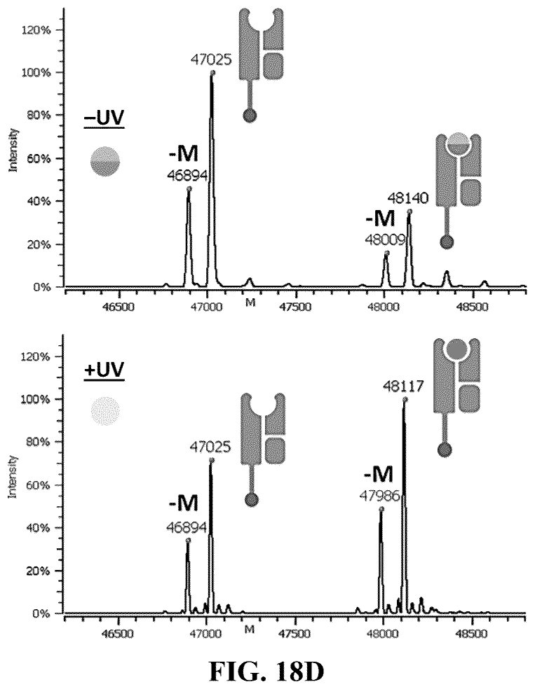

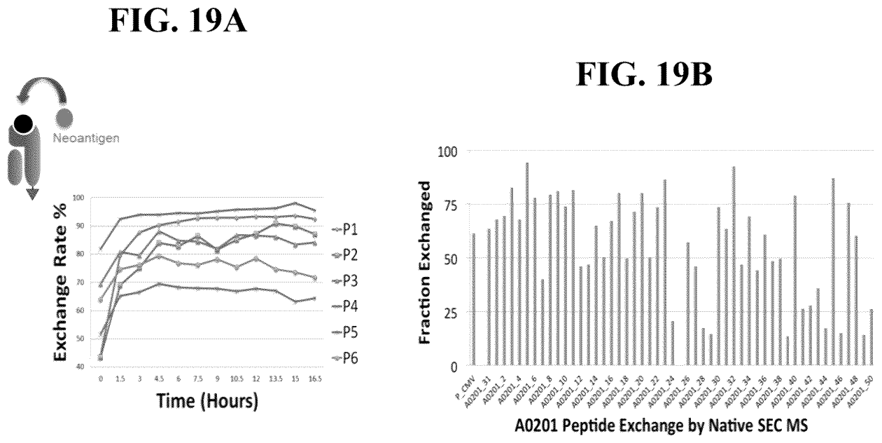

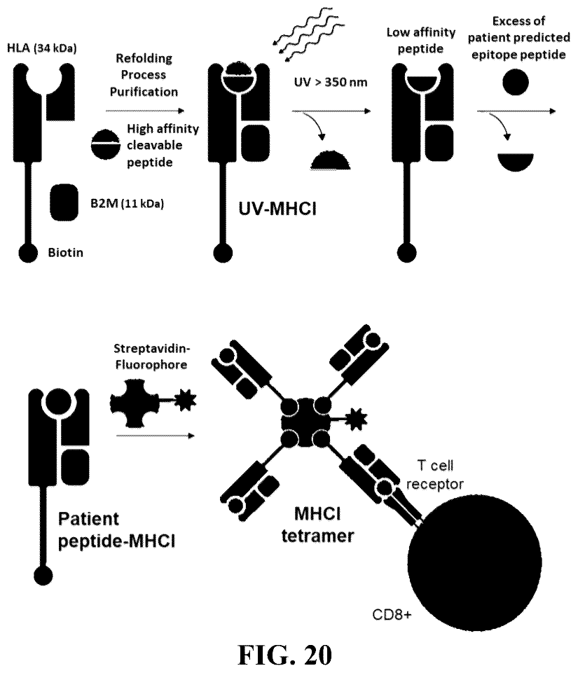

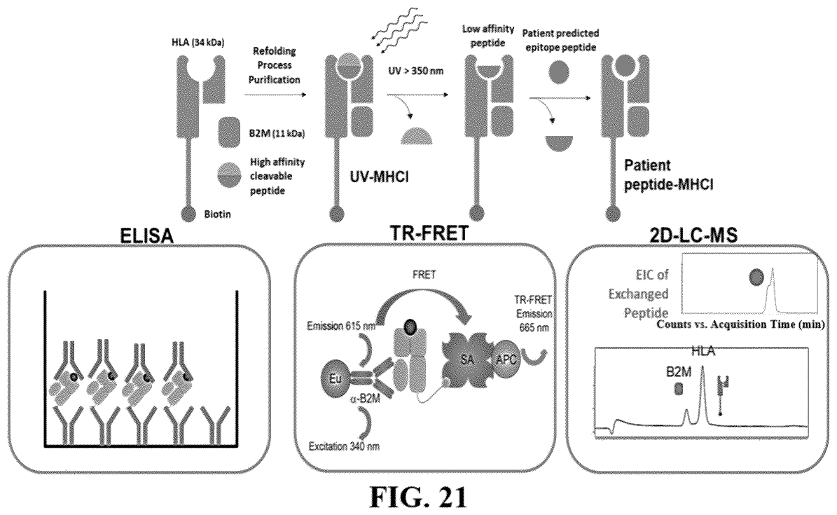

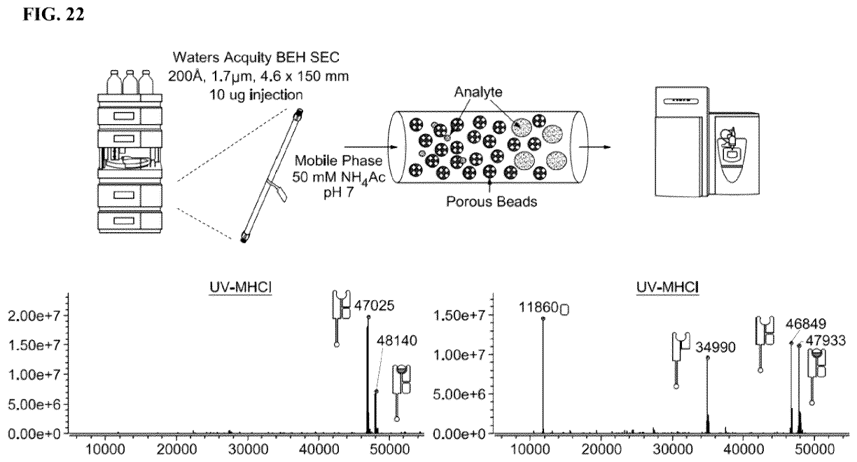

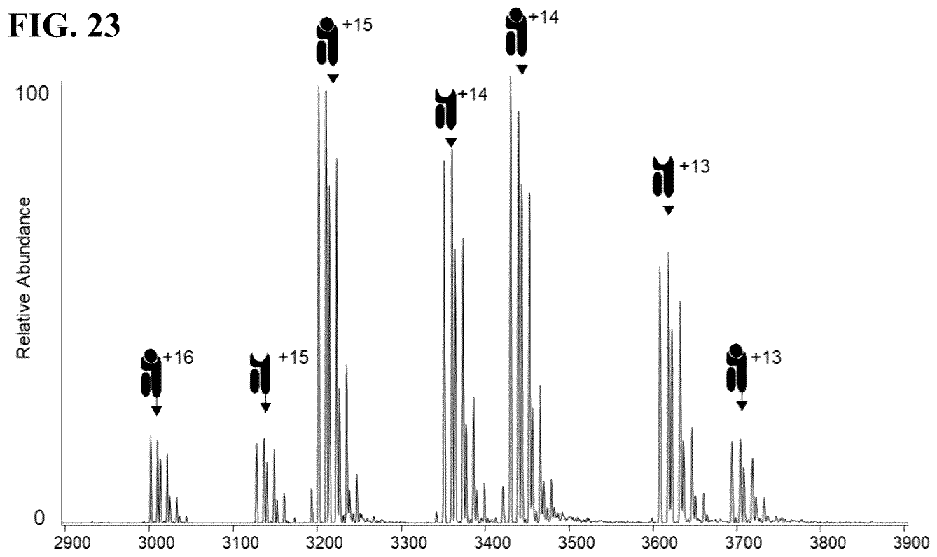

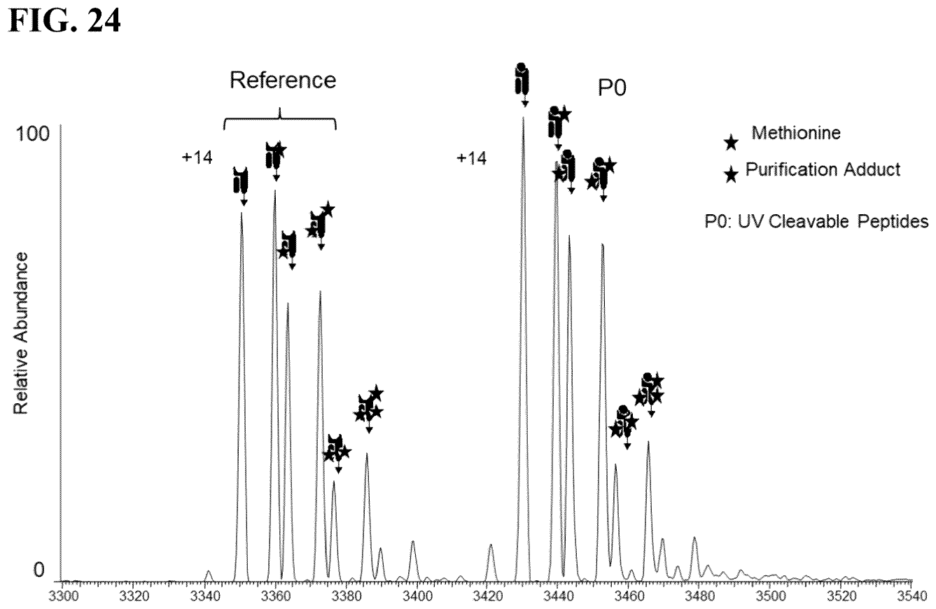

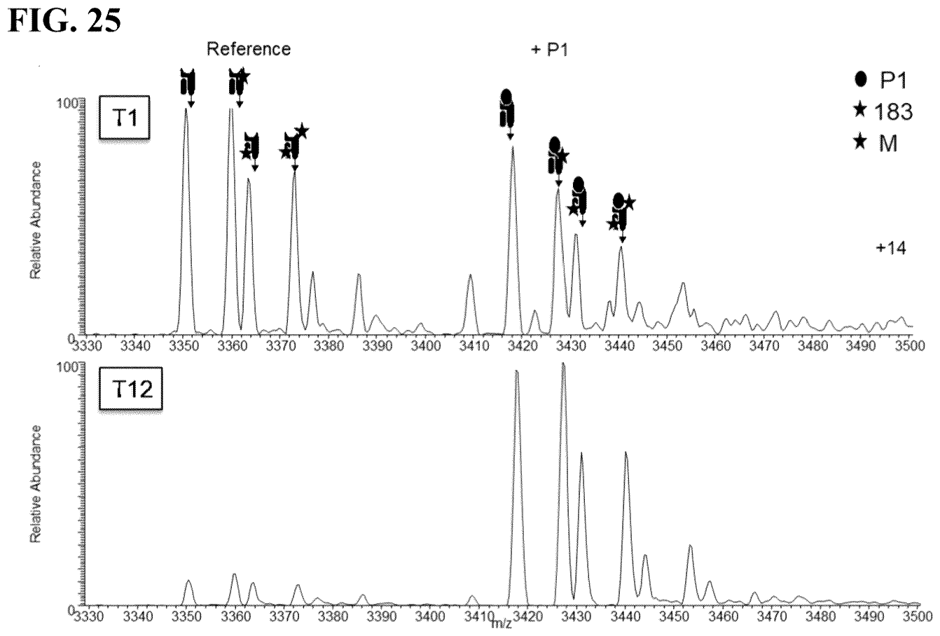

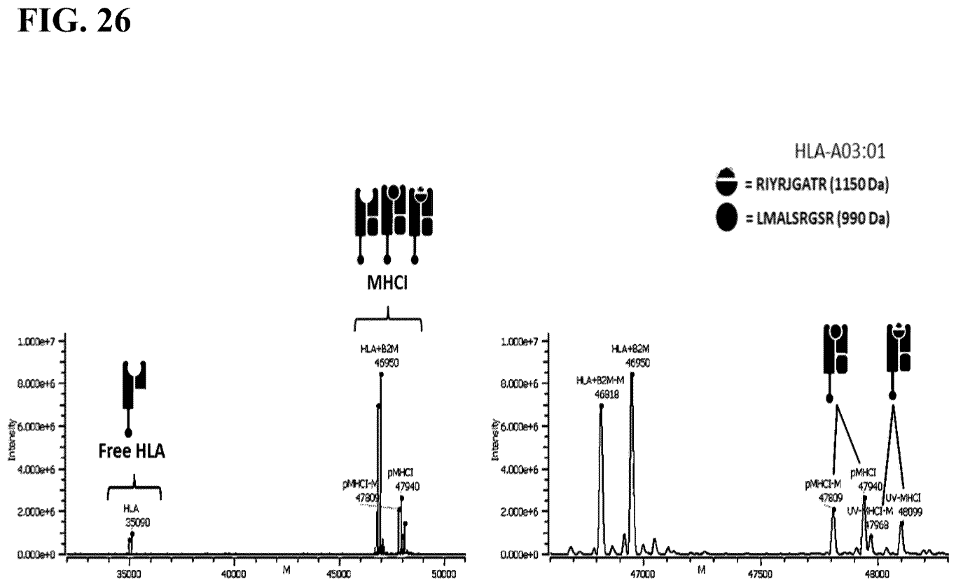

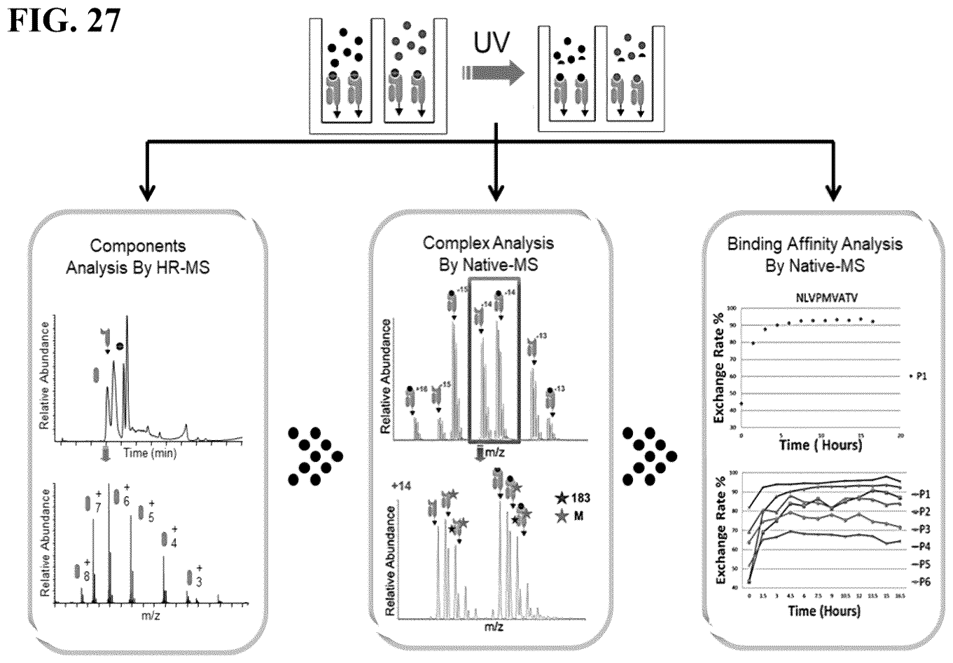

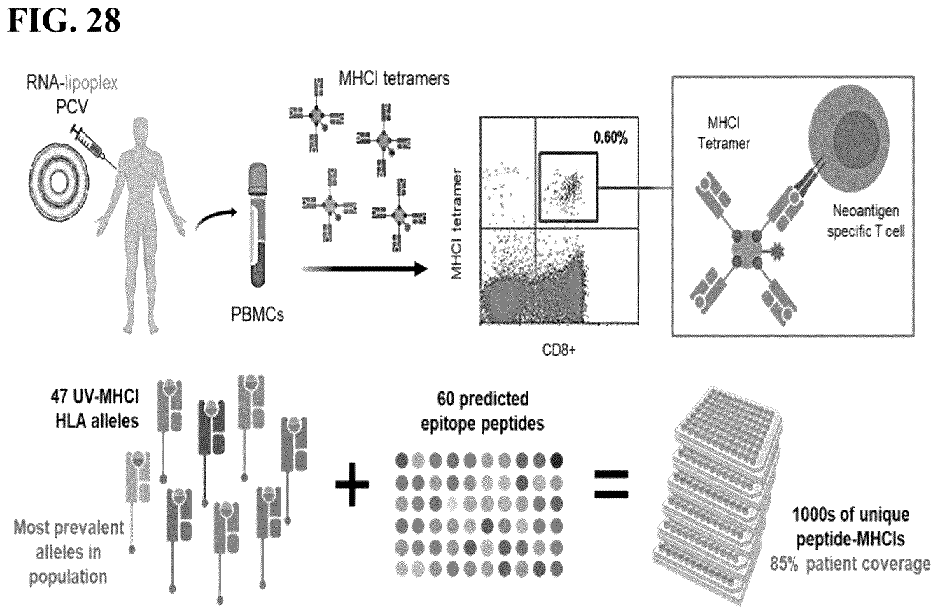

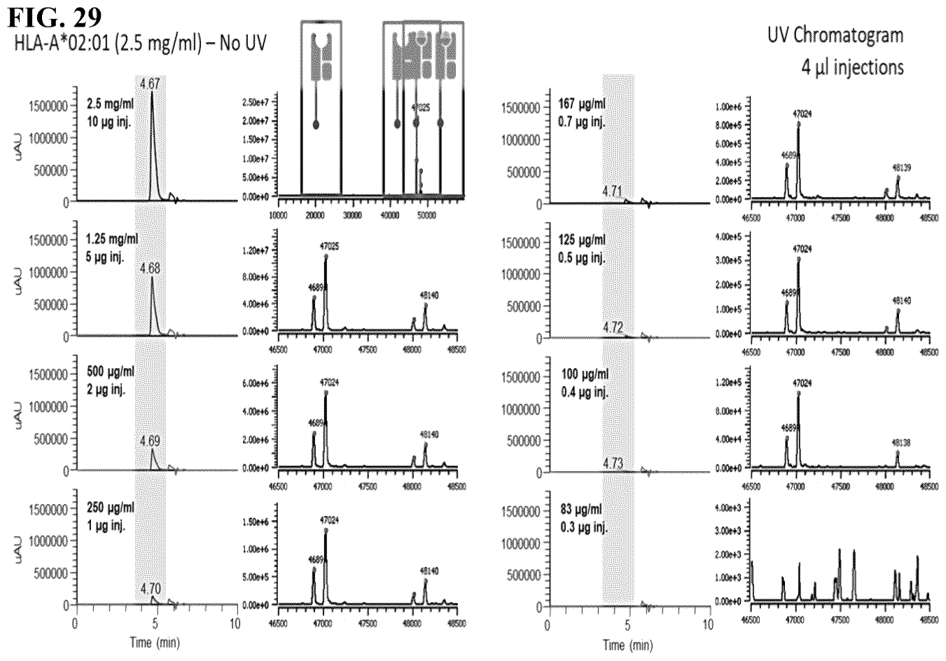

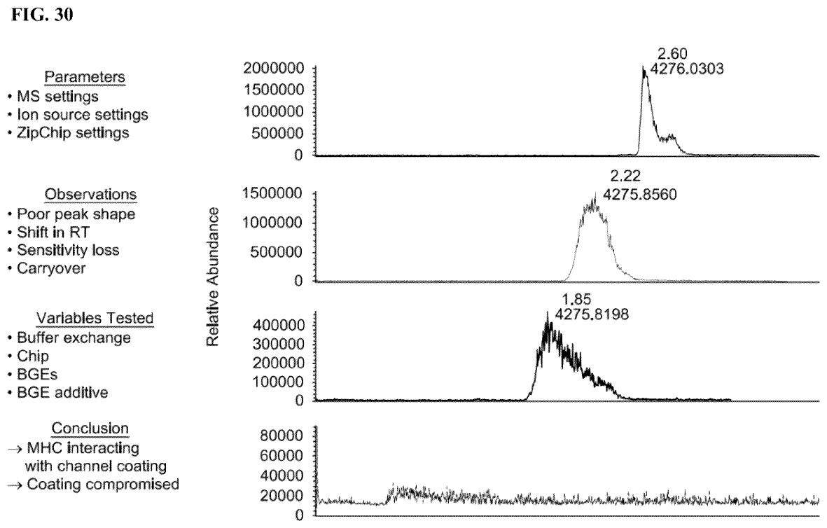

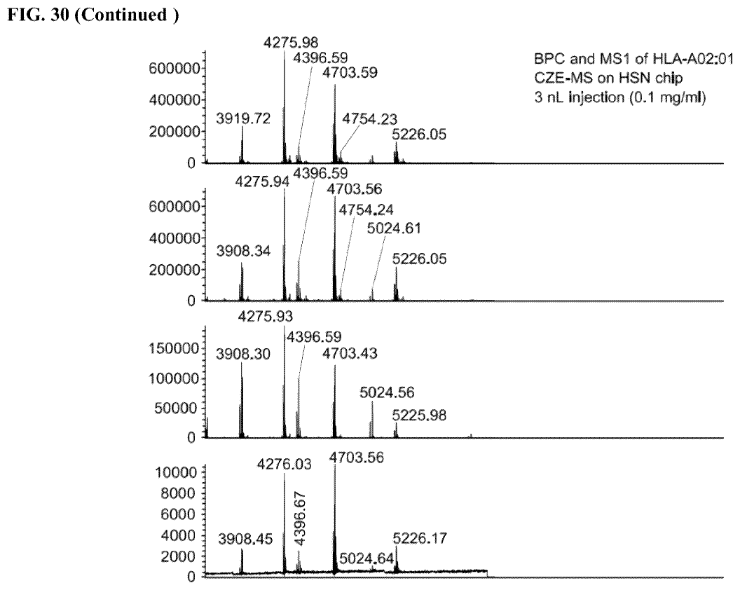

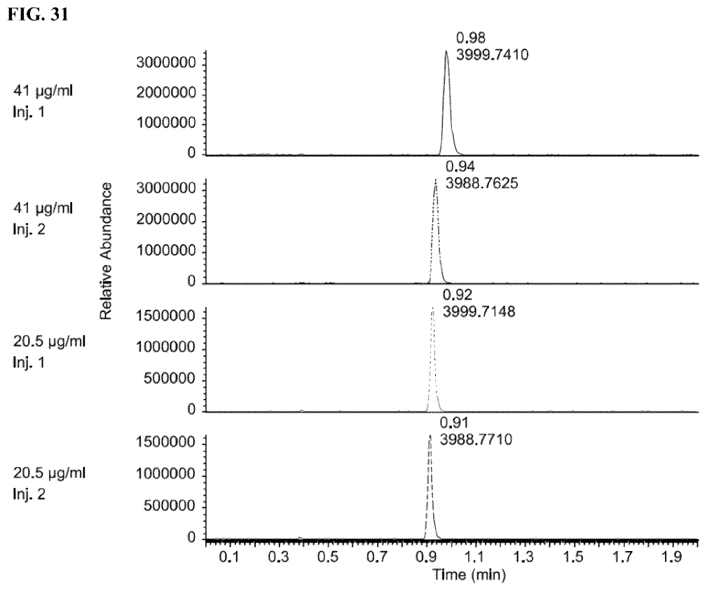

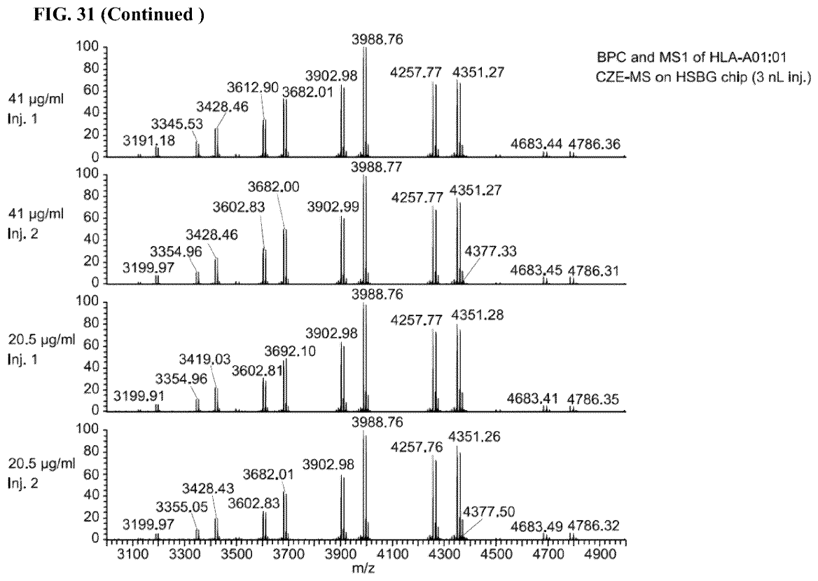

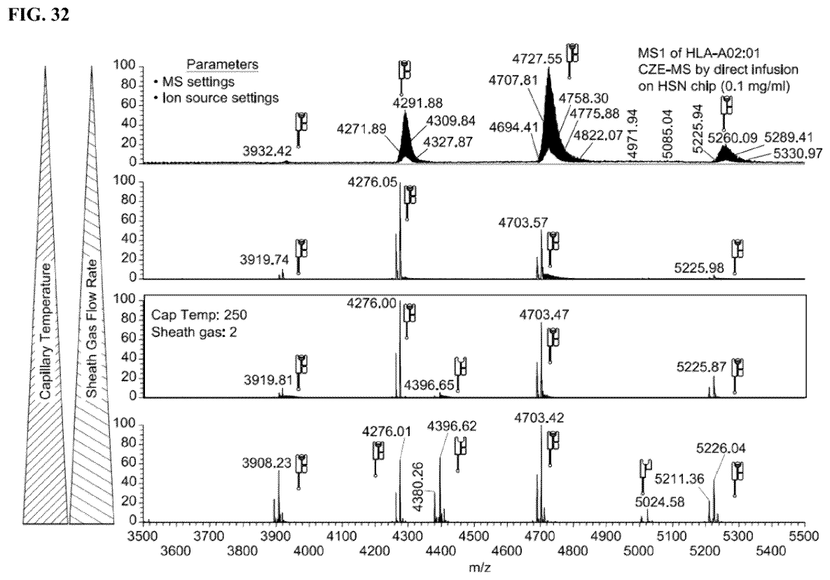

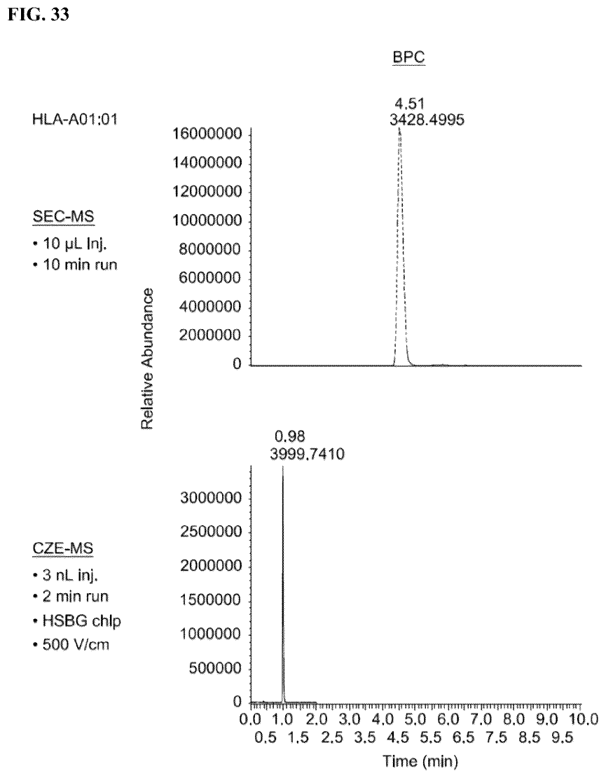

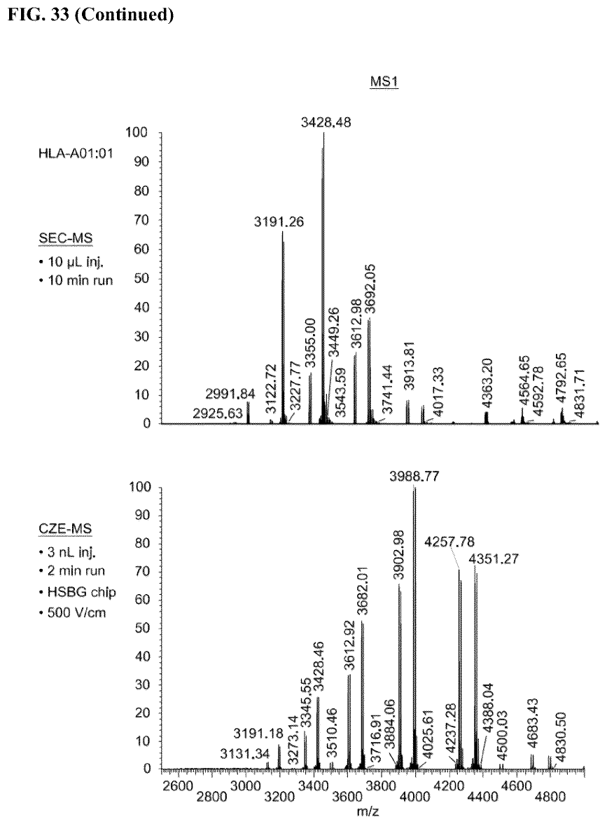

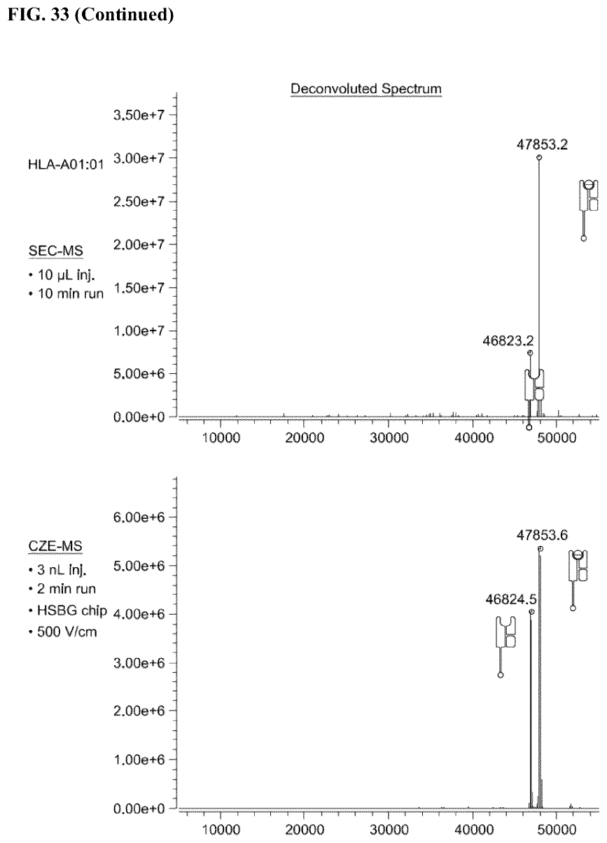

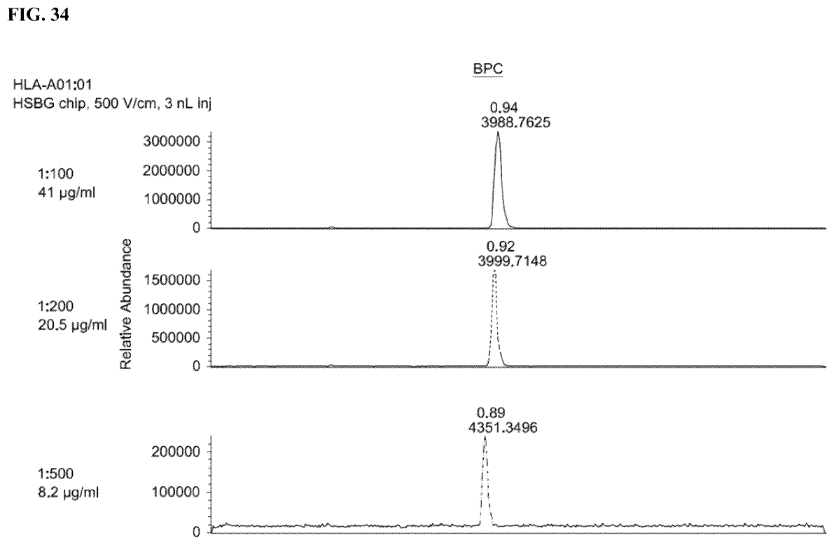

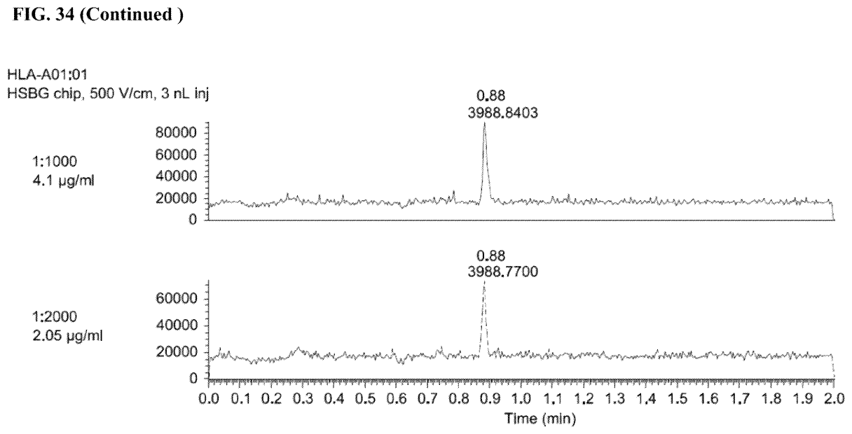

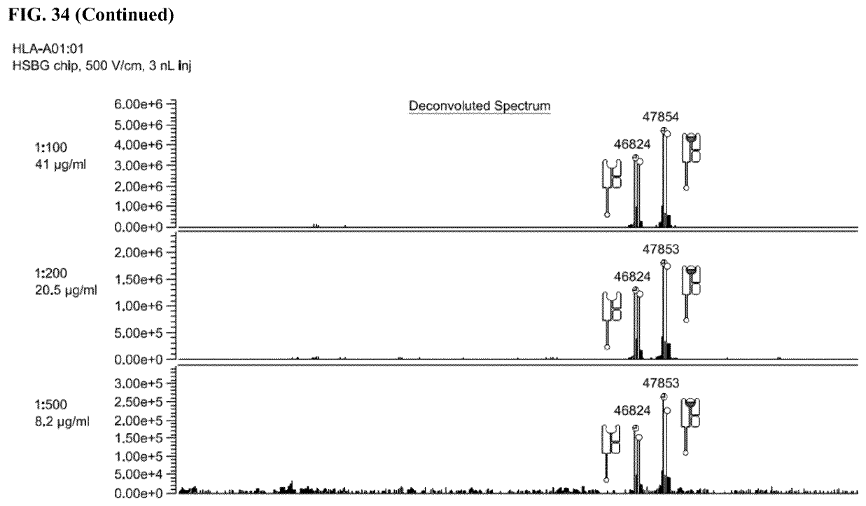

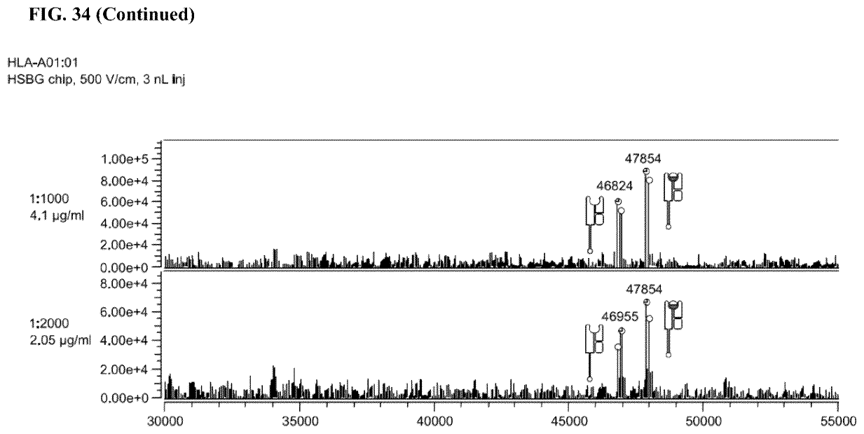

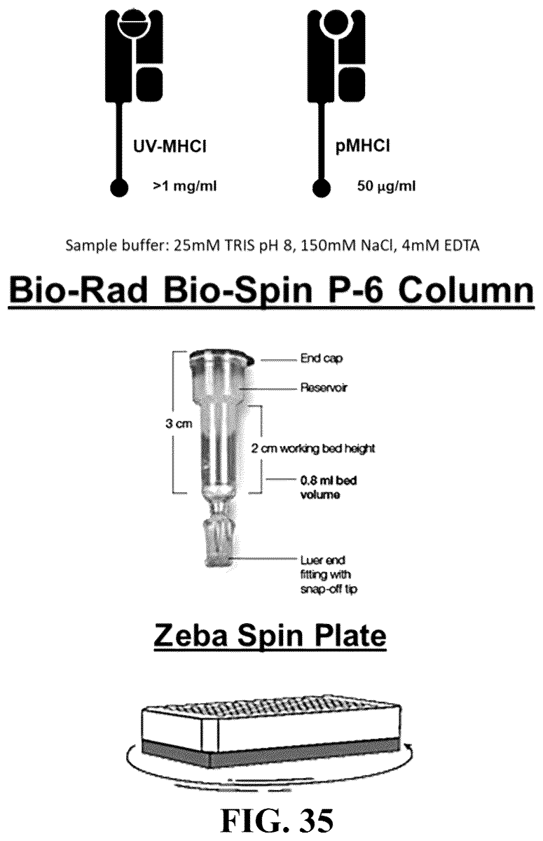

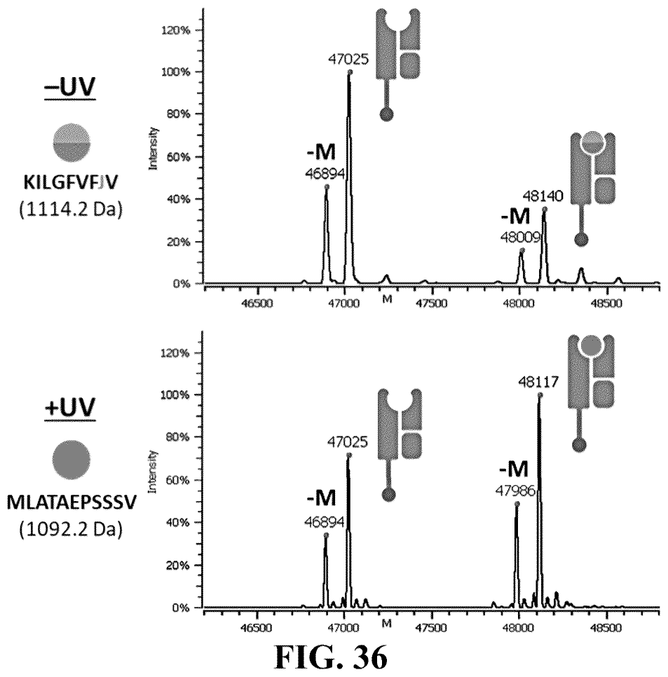

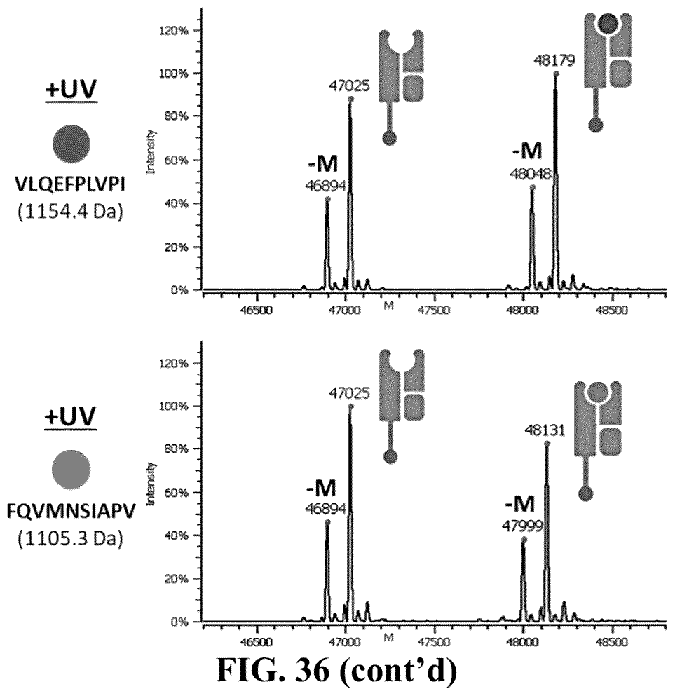

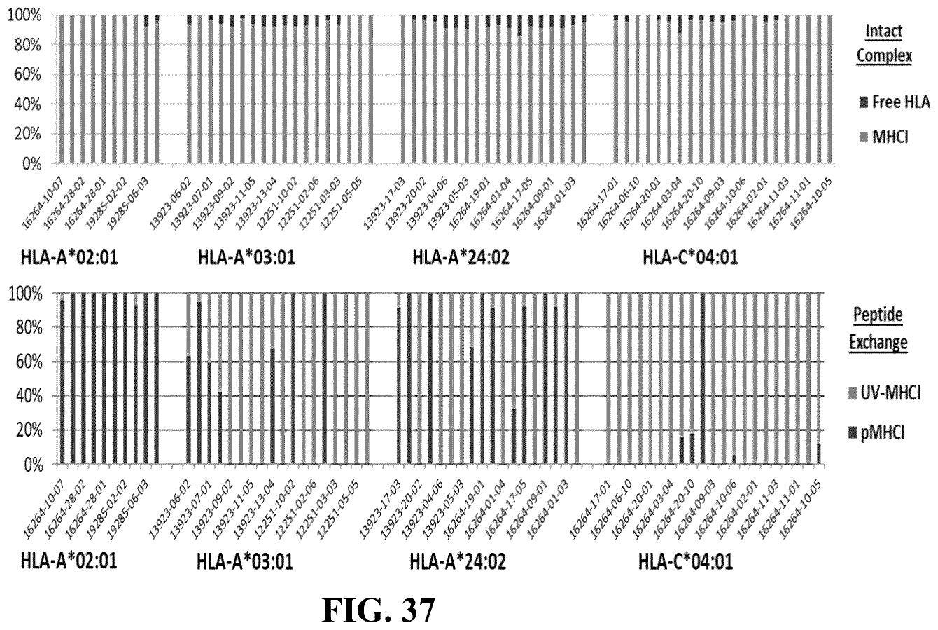

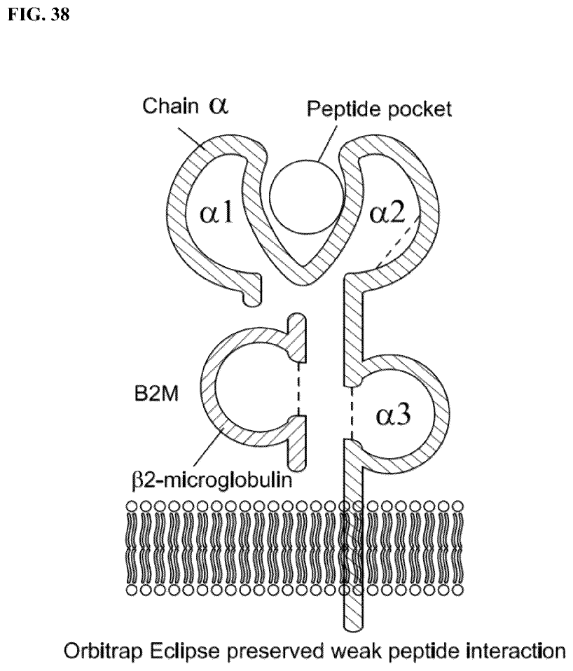

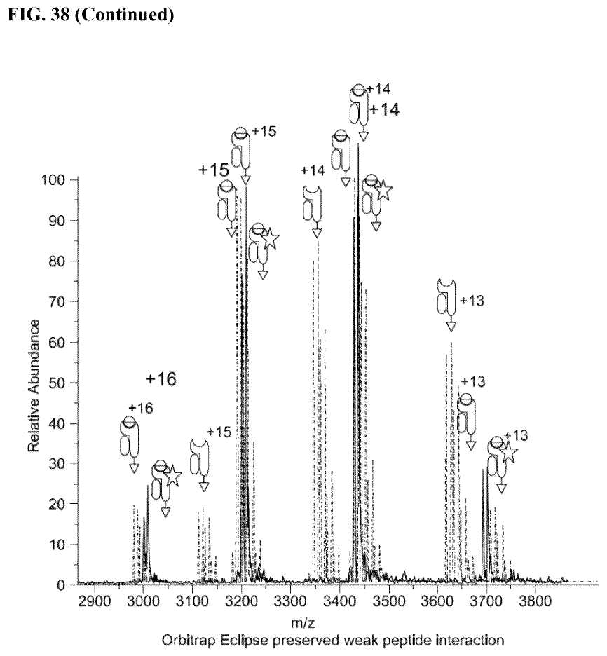

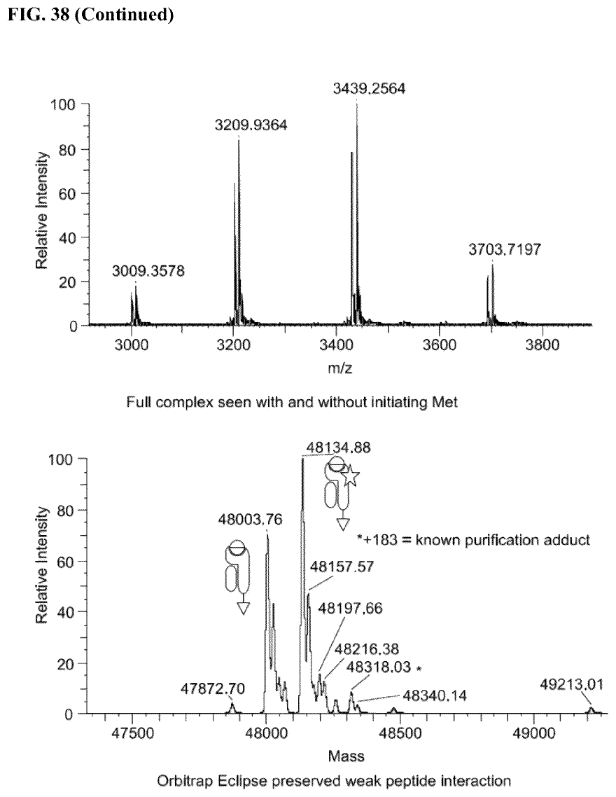

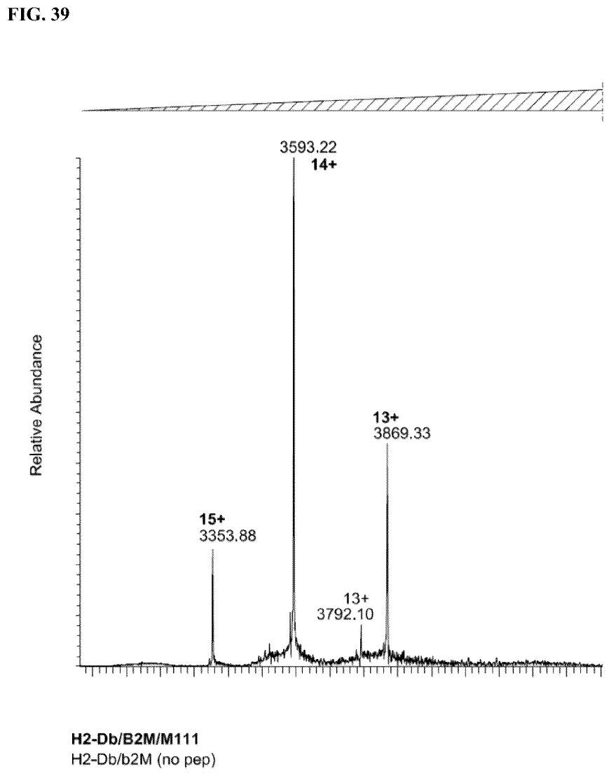

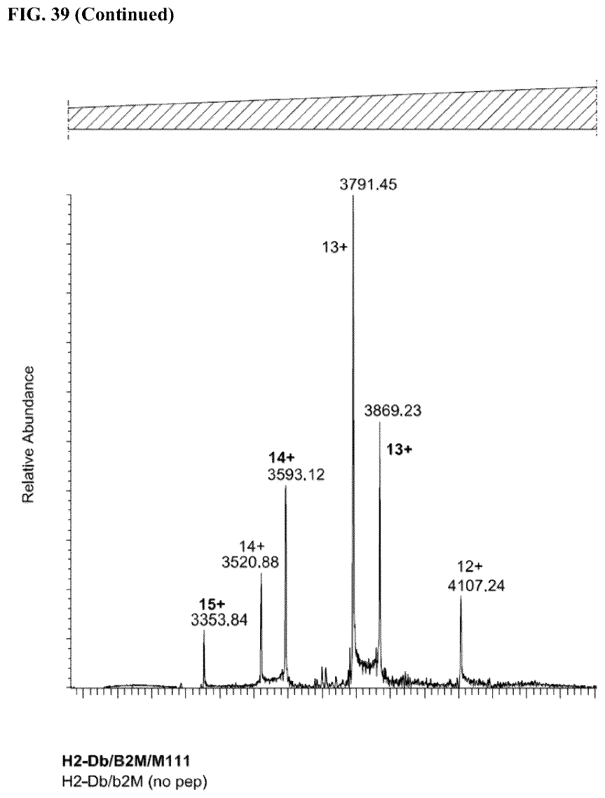

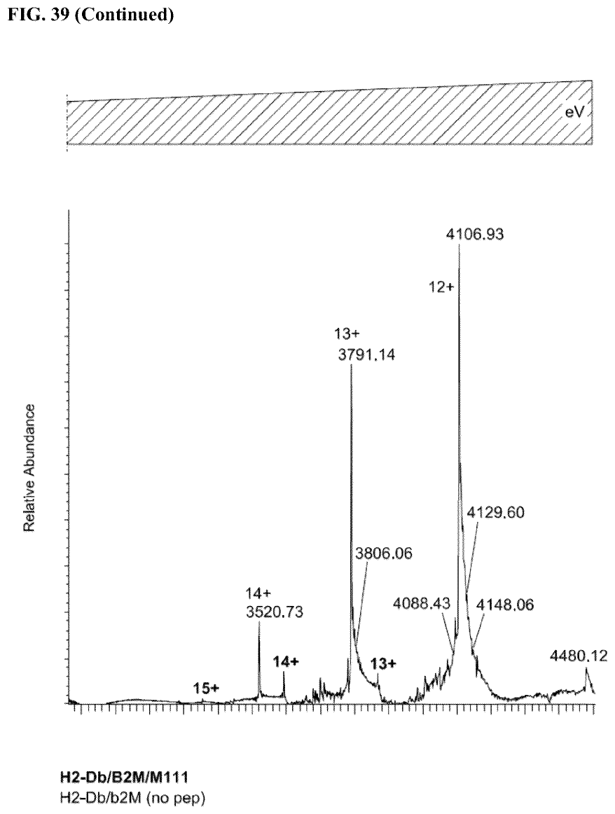

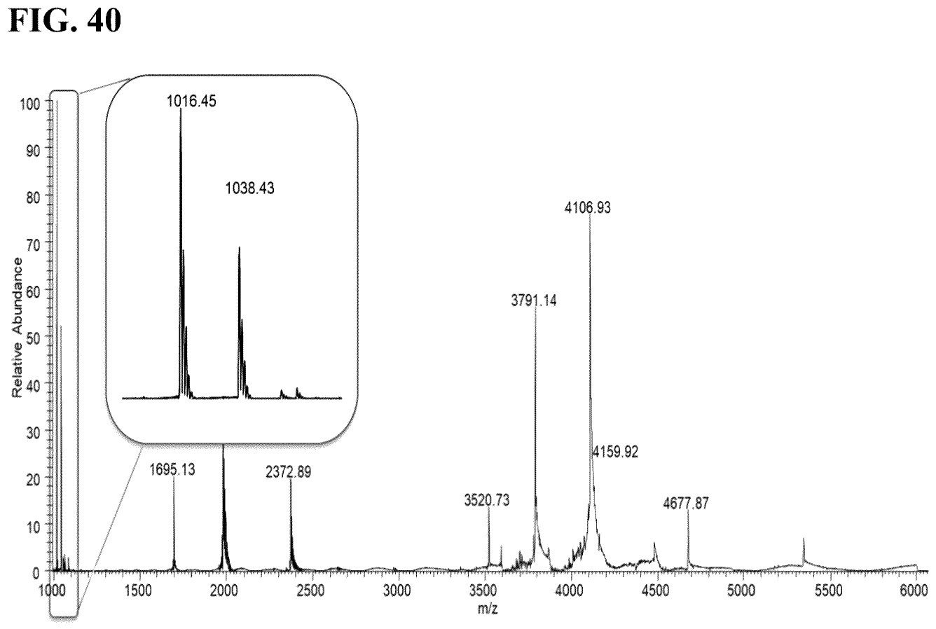

The MHCI/UV-cleavable peptide complex can undergo an UV-light exposure/peptide exchange assay in the presence of a pool of 40 peptides, as shown in FIG. 15. A sample collected from the exchange time course is run by 1) SEC and 2) LC-MS at various lengths of time (for example, between 5 and 10 hours, 40 min increments), the identity and intensity of the peptide from the complex peak isolated by SEC is measured by LC-MS. The plot shows the intensities of 10 peptides over time in an exchange reaction with MHCI HLA-A*01:01 over time. In total for this example run 25%, or 10, peptides from a pool of 40 peptides were determined to be true binders.

### Example 5: MHC Normalization Calculation

All data analysis was carried out using Genedata and Spotfire V7.8 unless indicated elsewhere.

For all MHC screens performed, control based normalization was used to ensure relative comparisons of the sample signal to MHC without peptide based on each allele and expressed as Delta F (%). The percentage of delta F was calculated using the following equation:

Delta F % (dF)=(RFU raw [pMHC]−RFU raw [negative control])/RFU raw [negative control]×100

Where RFU raw [negative control] is the TR-FRET signal ratio of 660 nm/615 nm from the wells of MHC monomer without peptide, which defines the minimal signal for hits selection for specific allele, RFU raw [pMHC] is the TR-FRET signal ratio of 660/615 from the wells of MHC monomer loaded with peptides.

For hits selection across alleles, robust Z score (RZ) was calculated from the normalized signal (dF) as the follows:

RZ score=([Sample]−median [all sample])/MAD

Where [sample] and median [all samples] are the Delta F value of sample and the median values of all samples in each 384 well plate, respectively. The MAD is the median absolute deviation of all samples in each 384 well plate.

For evaluate HTS assay performance, Z′ factor was calculated from the signal (RFU RAW) of positive and negative controls using the equation as below:

Z′=1−{(3σ RFU[pos]+3σ RFU[neg])/|μ RFU[pos]−μ RFU[neg]|}

Where 6 [pos] and 6 [neg] are the standard deviations and the μ [pos] and μ [neg] are the mean values of the positive and negative control, respectively. For RZ′ (robust Z′). Median absolute deviation (MAD) and median values were substituted for standard deviation and mean values.

### Example 6. Native SEC-MS

MHCI proteins were injected onto an ACQUITY UPLC Protein BEH SEC column (200 Å, 1.7 μm, 4.6 mm×150 mm, Waters Corporation) heated to 30° C. using an UltiMate™ 3000 RSLC system (Thermo Fisher Scientific). A binary pump was used to deliver solvent A (water) and solvent B (100 mM ammonium acetate, pH 7.0) as an isocratic gradient of 50% solvent B at a flow rate of 300 μL/min for 10 min. Separated proteins were analyzed online via electrospray ionization into a Thermo Exactive™ Plus EMR Orbitrap™ instrument (Thermo Fisher Scientific) using the following optimized parameters for data acquisition: sheath gas flow rate of 4 and AUX gas flow rate of 0 in ESI source; 4.0 kV spray voltage; 320° C. capillary temperature; 200 S-lens RF level; 350 to 10,000 m/z scan range; desolvation, in-source CID 0 eV, CE 0; resolution of 8,750 at m/z 200; positive polarity; 10 microscans; 3×106 AGC target; fixed AGC mode; 0 averaging; 50 ms maximum IT; 25 V source DC offset; 8 V injection flatapole DC; 7 V inter-flatapole lens; 6 V bent flatapole DC; 0 V transfer multipole DC tune offset; 0.8 V C-trap entrance lens tune offset; and trapping gas pressure setting of 3. (See Tables 1 and 2)

Acquired mass spectral data were analyzed using PMI Intact Mass™ software (Protein Metrics Inc.) under the following parameters: 1,500 to 6,000 m/z range; 0.2 charge vectors spacing; 15 m/z baseline radius; 0.02 m/z smoothing sigma; 0.04 m/z spacing; 3 mass smoothing sigma; 0.5 mass spacing; 10 iteration max; and 5 to 100 charge state range. Relative quantification was based on the intensity of each individual peak versus total summed intensities in the deconvoluted spectrum.

### Example 7: Native CZE-MS

MHCI proteins were buffer-exchanged using Zeba™ Spin Desalting Plate, 96-well (Thermo Scientific) prior to native CZE-MS analysis. The desalting plate was first equilibrated to room temperature and then centrifuged at 1,000×g for 2 min to remove the storage buffer. The resin was washed four times with 250 μL of 50 mM ammonium acetate, pH 7.0 by centrifuging at 1,000×g for 2 min. The wash plate was emptied after each spin and then replaced with a sample collection plate. Samples were added on the resin and centrifuged at 1,000×g for 2 min.

Buffer-exchanged MHCI proteins were injected onto an HS chip (908 Devices Inc.) using a ZipChip™ system (908 Devices Inc.). A ZipChip™ autosampler was used to deliver a protein complex background electrolyte (BGE) solution, pH 6.5, containing isopropyl alcohol, histidine, ammonium acetate, and dimethyl sulfoxide. The final ZipChip™ method was optimized with the following parameters: 500 V/cm field strength; 3 nL injection volume; 0.5 min pressure assist start time; 2 min replicate delay; and 3 min analysis time. Separated proteins were analyzed online via electrospray ionization into a Thermo Exactive™ Plus EMR Orbitrap™ instrument (Thermo Fisher Scientific) using the following parameters for data acquisition: sheath gas flow rate of 2 and AUX gas flow rate of 0 in ESI source; 0 kV spray voltage; 250° C. capillary temperature; 200 S-lens RF level; 1,500 to 6,000 m/z scan range; desolvation, in-source CID 75 eV, CE 0; resolution of 17,500 at m/z 200; positive polarity; 3 microscans; 3×10⁶ AGC target; fixed AGC mode; 0 averaging; 20 ms maximum IT; 15 V source DC offset; 9 V injection flatapole DC; 8 V inter-flatapole lens; 10 V bent flatapole DC; 0 V transfer multipole DC tune offset; 0 V C-trap entrance lens tune offset; and trapping gas pressure setting of 2. (See Tables 1 and 2).

Acquired mass spectral data were analyzed using PMI Intact Mass™ software (Protein Metrics Inc.) under the following parameters: 1,500 to 6,000 m/z range; 0.2 charge vectors spacing; 15 m/z baseline radius; 0.02 m/z smoothing sigma; 0.04 m/z spacing; 3 mass smoothing sigma; 0.5 mass spacing; 10 iteration max; and 5 to 100 charge state range. Relative quantification was based on the intensity of each individual peak versus total summed intensities in the deconvoluted spectrum.

### Example 8: Peptide Exchange Process: Pre-CZE-MS

UV-MHCIs were produced by refolding HLA and B2M subunits with a high affinity, synthetic peptide containing a UV-cleavable, non-natural amino acid. Upon UV exposure, the peptide is cleaved, rendering it to lose its affinity and allowing it to be exchanged with an excess of patient-predicted epitope peptides to form peptide-exchanged MHCIs (pMHCIs). Peptide-exchanged MHCIs were then assembled into tetramers for T-cell staining for immune response monitoring. To validate peptide exchanges in their non-covalent, intact forms, SEC-MS under native conditions was first evaluated with UV-MHCI samples with concentrations greater than 1 μg/μl. SEC-MS analysis of UV-MHCIs successfully demonstrated the stability of these non-covalent complexes. Before UV treatment, only intact UV-MHCI complexes were observed. After UV treatment, the complex falls apart and the main mass peaks observed were HLA and B2M subunits. However, the limit of detection of UV-MHCI was below 0.3 μg, rendering peptide-exchanged MHCIs that were 50 ng/μl after saturation with peptide of interest unsuitable for validating peptide-exchanges. To circumvent the sensitivity limitation while maintaining the ability to analyze by native mass spectrometry, these peptides were detected and analyzed by CZE instead using the ZipChip™ CZE system.

### Example 9: High-Throughput Identification of Conditional MHCI Ligands and Scaled-Up Production of Conditional MHCI Complexes

Here is a novel workflow developed to enable the identification and validation of peptides containing a non-natural UV cleavable amino acid (conditional MHCI ligands) that form stable MHC complexes across a range of HLA alleles for use in the high-throughput (HTP) generation of MHCI monomers and tetramers via peptide exchange. Known peptide binders identified in the Immune Epitope Database (IEDB) were screened by forming stable refolded MHCI complexes and MHCI/peptide complexes were detected via an ELISA assay. Conditional MHCI ligands were designed using the highest-performing peptides from the initial ELISA screen. The conditional MHCI ligands were then evaluated in the same ELISA assay and the top performers were selected for scale-up production. A novel MHCI purification and biotinylation protocol was developed to enable large-scale production (15 L) using the known conditional MHCI ligand for the HLA-A*02:01 allele, which yielded >60 mg of MHCI in a single refold production run. Next-generation analytical techniques were also used to characterize the refolded complex, including LC/MS, SEC-MALS and 2D LC/MS. The optimized refold production protocol and next-generation analytical techniques were applied to generate MHCI complexes with the conditional MHCI ligands identified in the ELISA screen and were found to be properly refolded and of high purity and quality. Finally, the extent of peptide exchange after UV exposure was evaluated with validated peptide binders using 2D LC/MS for the MHCI complexes generated with the new conditional MHCI ligands. Successful peptide exchange was observed for all conditional MHCI ligands upon UV exposure in the presence of a peptide binder. These combined results demonstrate that the workflow described in this study can be used to identify conditional MHCI ligands for new HLA alleles. This approach has the potential to be broadly applied and enable HTP generation of MHCI monomers and tetramers across a wider range of HLA alleles, which could be critical to enabling the use of MHCI tetramers to monitor neoantigen-specific T-cells in the clinic.

Over the past ten years, Major Histocompatibility Complex Class I (MHCI) presentation of cancer neoantigens has emerged as the critical mode of action by which our immune system can control tumor growth. Because of the role neoantigen-specific T-cells play in killing tumors, significant resources across academia and biotechnology have been dedicated to developing clinically active drugs that will amplify the cancer immunity cycle and improve the magnitude and breadth of the neoantigen specific T-cell responses, including checkpoint inhibitors, cytokines, TNF superfamily agonists, cancer vaccines and immune modulators. Despite these drug development efforts, there are very limited tools available to monitor the impact of treatment on neoantigen-specific T-cell responses (T-cell phenotype, T-cell magnitude and breadth, epitope spreading, etc.).

The most common methods for tracking T-cell responses are ELISPOT and MHC tetramer staining. The ELISPOT assay is a functional assay that measures cytokine release from T-cells upon stimulation of PBMCs with antigens. The benefits of this assay are that it is allele- and neoepitope-independent (i.e. only the neoantigen needs to be known) and it is a functional readout. The drawbacks to the assay are that it is semi-quantitative and there is no way to assess T-cell phenotype, which could be critical to understanding factors important to generating a protective immune response. MHCI tetramer-based detection utilizes recombinant MHCI monomers multimerized into tetramers via streptavidin conjugation as neoantigen-specific T-cell staining reagents. This method allows for staining of multiple specificities as well as phenotype markers. MHCI tetramers also allow for quantitative analysis of the exact number of neoantigen-specific T-cells and how this may change during the course of treatment. Therefore, in many respects MHCI tetramer-based detection can provide a more detailed understanding of the effect of treatment on the neoantigen-specific CD8+ T-cell response.

Despite the advantages of MHCI tetramer detection, this approach has not been widely adopted as a biomarker strategy across clinical programs because of challenges associated with generating the reagent. MHCI tetramers require a time-consuming and difficult multi-day refold process including multiple chromatography steps for reagent generation. In addition, the neoantigen profile is unique to each patient and many patient-specific MHCI tetramers would be required to gain a complete picture of the T-cell landscape in a given patient. Furthermore, the HLA allele is highly polymorphic (nearly 20,000 HLA class I alleles exist), and each human has six different HLA alleles. Therefore, MHCI tetramer-based detection of neoantigen specific T-cell responses would require implementation of a personalized MHCI tetramer platform, which is not possible using traditional MHCI generation protocols.

To address these limitations, Rodenko et al developed a rapid high-throughput (HTP) method for the generation of MHCI monomer and tetramers. This method involves generating MHCI complexes with a UV-cleavable peptide that binds with high affinity when intact and low affinity when cleaved (conditional MHCI ligands). This functionality enables peptide exchange upon UV exposure when MHCI complexes assembled with conditional MHCI ligands (conditional MHCI complexes) are incubated in the presence of a high-affinity peptide binder of interest. Conditional MHCI complexes for a given HLA allele can be refolded at large scale, and the end user can then exchange the conditional MHCI ligand for any other peptide of interest. This invention provided a breakthrough in terms of enabling the use of personalized MHCI tetramers to monitor neoantigen specific T-cells in the clinic. However, conditional MHCI ligands are specific for each HLA allele and must be identified de novo. To the best of our knowledge, conditional MHCI ligands have only been published for 24 HLA alleles. Although these alleles are some of the most prevalent, neoantigen coverage across a broad cohort of diverse patients will still be minimal. Therefore, there is a need to develop workflows to enable the expansion of allele coverage.

In addition, analytically validating MHCI complexes after refolding or peptide exchange has utilized a limited number of analytical techniques including ELISA assays and gel electrophoresis. Although these techniques have proven useful to determine if the MHCI complex is present and for semi-quantitative analysis of affinity and stability, several other important parameters, such as HLA:B2M ratio, aggregation, oxidation state and native condition analysis, are not captured using this type of analysis. Several protein analytical tools exist to evaluate these parameters, including liquid chromatography/mass spectrometry (LC/MS), 2D LC/MS and size exclusion chromatography-multiple angle light scattering detection (SEC-MALS), yet very little has been done to apply these tools to the characterization of MHCI complexes after refold or peptide exchange.

An experimental workflow was developed at allows for the identification and validation of new combinations of conditional MHCI ligands and HLA alleles that form stable conditional MHCI complexes. An ELISA assay was developed and validated for detection of stable conditional MHCI complexes. Five published peptide binders reported in the Immune Epitope Database and Analysis (IEDB) were initially screened across six HLA alleles (A*02:03, A*26:01, B*18:01, B*35:03, C*02:02, C*14:02) and designed conditional MHCI ligands were based on the top binders. The conditional MHCI ligands were then screened in the ELISA assay, and the top performers were selected for scale-up production. For MHCI production, a novel MHCI purification and biotinylation protocol was developed and next-generation analytical techniques were used to confirm the quality of complexes generated. The optimized protocol and analytical techniques were applied to produce and characterize the conditional MHCI complexes generated with the newly identified conditional MHCI ligands. The extent of peptide exchange after UV exposure was evaluated with validated peptide binders using 2D LC/MS. Thus a validated workflow was developed to identify conditional UV peptides for new HLA alleles that can be broadly applied in order to greatly expand HLA allele coverage, which could be critical to enabling the use of MHCI tetramers to monitor neoantigen-specific T-cells in the clinic.

Protein Expression and Purification.

Recombinant HLA and B2M were over expressed in E. coli, purified from inclusion bodies, and stored in denaturing buffer (6M Guanidine HCl, 25 mM Tris pH 8) at −80° C. After induction of expression, B2M and HLA biomass pellets were resuspended in lysis buffer (PBS+1% Triton X-114) at 5 mL/g and homogenized twice in a microfluidizer at 1000 bar. The homogenized suspension was then spun at 30000 g for 20 min in an ultracentrifuge. The pellets were collected, washed with 500 mL of 0.5% Triton X-114 in PBS and centrifuged at 30000 g for 20 min. The pellet was collected again and washed a second time as described above. The purified inclusion bodies were dissolved in a denaturing buffer (20 mM MES, pH 6.0, 6M Guanidine HCl) at a concentration of 10 mL/g and stirred at 4° C. overnight. The dissolved pellet was centrifuged at 40000 g for 60 min and the supernatant was collected and filtered through a 0.22μ filter. The concentration was determined using a BCA assay. Samples were then snap-frozen and stored at −80° C. prior to generation of MHCI complexes.

Peptide Selection for Screening.

Peptides for the initial binding screens were selected from the Immune Epitope Database and Analysis Resource (www.iedb.org). The peptide binders identified in the database were sorted based on experimentally measured affinity, and five peptides with the highest measured affinity were selected. In cases where the peptide sequences were similar for the top five (differing by less than four amino acids), the next highest affinity peptides with unique sequences were selected to ensure maximal peptide diversity in the screen.

MHCI Refold (Small Scale).

Recombinant HLA alleles and B2M were over-expressed in E. coli, purified from inclusion bodies, and stored under denaturing conditions (6M Guanidine HCL, 25 mM Tris pH 8) in −80° C. as described above. In a 200 μL reaction, the peptide (0.01 mM, per well), oxidized and reduced glutathione (0.5 mM and 4.0 mM, respectively), recombinant HLA alleles (0.03 mg/mL) and B2M (0.01 mg/mL) were all combined within a 96-well plate. Refold screens were performed with five different peptides for each HLA of interest as described above (Table 3). and the MHCI complex was incubated at 4° C. for 3-5 days to allow refolding. Refolded samples were analyzed by ELISA, and the highest performing peptide was selected for further analysis. All data was analyzed relative to the negative control refold, which was performed in the absence of peptide (labeled NP). HLA-A*02:01 refolded with the CMV pp65 viral epitope was used as a positive control.

After identifying the most stable peptide binder for each HLA allele, peptides were redesigned with a UV-cleavable amino acid (denoted “J”) at different positions along the peptide sequence (Table 4). In brief, variants of the most stable peptide binders identified in the initial screen were redesigned in which the J amino acid was substituted at positions 2, 4, 6, and 8, relative to the N-terminus. Formation of stable conditional MHCI complexes upon refolding with the redesigned peptides were identified by ELISA as described above. The conditional MHCI ligands resulting in the most stable complex based on the ELISA assay readout were used for scaled-up MHCI production. The original peptide (containing no UV amino acid substitution) was used as a positive control. All peptides used here were purchased from JPT (www.jpt.com) or ELIM Biopharm (www.elimbio.com).

ELISA Assay.

A 384-well Nunc Maxisorp plate (ThermoFisher Scientific, Waltham, MA) was coated with 25 μL/well of mouse IgG2a anti-HLA ABC clone W6/32 (Novus Biological, Littleton, Co.) at 8 μg/mL in coating buffer (0.05 sodium carbonate pH 9.6). After overnight incubation at 4° C., the plate was washed 3 times with wash buffer (PBS, 0.5% Tween 20). The plate was then blocked with 50 μL/well of Block buffer (PBS, 0.5% BSA, 10 ppm Proclin) and incubated at room temperature (RT) with agitation for 1 hour. After washing the plate 3 times with wash buffer, 25 μL/well of the unpurified refolded MHC complex at 40 μg/mL with and without peptides in assay diluent (PBS, 0.5% BSA+0.05% Tween20+10 ppm Proclin) was added to the plate and incubated for 1 hour at RT. The plate was washed six times and 25 IL of biotinylated mouse IgG1 anti-human B2M (BioLegend, San Diego, CA) at 100 ng/mL in assay diluent was added to each well. After a 1 hour incubation at RT and six washes, 25 μL/well of Streptavidin-Horseradish Peroxidase (GE, Marlborough, MA) was added to the plate and incubated for 30 min at RT. The color reaction was developed with TMB peroxidase substrate (Moss, Pasadena, MD) at RT for 15 min, and the reaction was stopped with 1M phosphoric acid. The absorbance was measured at a wavelength of 405 nm with a reference at 620 nm. Refolded MHC monomers without peptide or irrelevant peptide were used as the negative controls for each allele to normalize the signal.

MHCI Refold, Biotinylation, and Purification (Large Scale).

In a 1, 5 or 15 L reaction, the selected peptide (0.01 mM), oxidized and reduced glutathione (0.5 mM and 4.0 mM, respectively), recombinant HLA (0.03 mg/mL) and B2M (0.01 mg/mL) were combined in refold buffer (100 mM Tris, pH 8.0, 400 mM L-Arginine, 2 mM EDTA). The refold mixture was then stirred for 3-5 days at 4° C., filtered through a 0.22 μm filter, and concentrated and buffer exchanged by tangential flow filtration (TFF) (Millipore P2C010001) into 25 mM Tris pH 7.5. The protein components were analyzed by LC/MS to ensure that the HLA was in the appropriate reduced state. The concentrated and refolded MHCI complex was then biotinylated through the addition of BirA (1:50 (wt:wt) enzyme:MHCI), 100 mM ATP and 10×reaction buffer (100 mM MgOAc, 0.5 mM biotin). The biotinylation reaction was mixed for 2 hr at room temperature. The sample was dialyzed and analyzed by LC/MS to quantify biotinylation. The biotinylated MHCI complex was purified by anion exchange chromatography using a 1 or 5 mL HiTrap Q HP column, depending on the reaction size, on an AKTA Avant FPLC. The column was equilibrated with 10 column volumes (CV) of 25 mM Tris HCl pH 7.5 at a flow rate of 5 mL/min. The MHCI complex was loaded on the column at a 5 mL/min flow rate and eluted using 0-60% 2.5 mM Tris HCl, pH 7.5, 1 M NaCl gradient over 30 CV. Fractions across the eluted peak were run on SDS-PAGE, and fractions containing both B2M and HLA bands were pooled. Pooled fractions were buffer-exchanged into storage buffer (25 mM Tris HCl, pH 8.0, 150 mM NaCl). Protein concentration was determined by UV absorbance at 280 nm, and samples were snap-frozen and stored at −80° C.

LC/MS Analysis.

Between 2 and 5 μg of MHCI complex were injected on an AdvanceBio RP-mAb diphenyl column, 2.1×75 mm, 3.5 μm (Agilent). The column was heated to 80° C. and exposed to a gradient of 25-40% mobile phase B in 2.0 min at 0.8 mL/min. Mobile phase A was 0.05% TFA in water. Mobile phase B was 0.05% TFA in acetonitrile. The column eluent was sent to an Agilent 6230 ESI-TOF LC/MS for mass spectrometry data acquisition.

In order to quantitate MHCI concentration and molar ratios of B2M to HLA, standard curves of B2M and HLA alleles were generated by injecting known amounts of each protein using the method described above. Peak areas at A280 were used to generate standard curves that allowed for the quantitation of the individual protein subunits in MHCI complexes. HLA and B2M masses were deconvoluted using the MassHunter Qualitative Analysis software (Agilent).

SEC VIALS Analysis.

The MW of the MHCI complex was determined as described previously. Briefly, samples were injected onto a TSKgel SW3000 analytical SEC column (Tosoh Bioscience), with isocratic gradient of phosphate buffered saline (PBS) (with an additional 150 mM NaCl), coupled to a multi-angle light scattering system (MALS) (Wyatt Instruments) to measure molar mass.

2D LC/MS analysis: A 2-dimensional liquid chromatography mass spectrometry (2D LC/MS) method was used to characterize peptide binding to MHCI complexes. Between 2-3 μg of MHCI complexes were injected on the instrument and sent to the first dimension column. The first dimension LC method employed an analytical size exclusion column (SEC) (Agilent AdvanceBio SEC 300 Å, 2.7 μm, 4.6×15 mm) to separate intact complex from excess peptide run at an isocratic flow of 0.7 mL/min in 25 mM TRIS pH 8.0, 150 mM NaCl for 10 min with signal acquisition at 280 nm. A sampling valve collected the entirety of the complex peak that eluted between 1.90-2.13 min in a volume of 160 μL and injected it onto the second dimension reversed phase column (Agilent PLRP-S 1000 Å, 8 μm, 50×2.1 mm). The second dimension column was exposed to a gradient of 5-50% mobile phase B in 4.7 min at 0.55 mL/min with the column heated to 80° C. Mobile phase A was 0.05% TFA. Mobile phase B was 0.05% TFA in acetonitrile. The column eluent was sent to an Agilent 6224 ESI-TOF LC/MS for mass spectrometry data acquisition.

ELISA-Based Analysis of MHCI Refold.

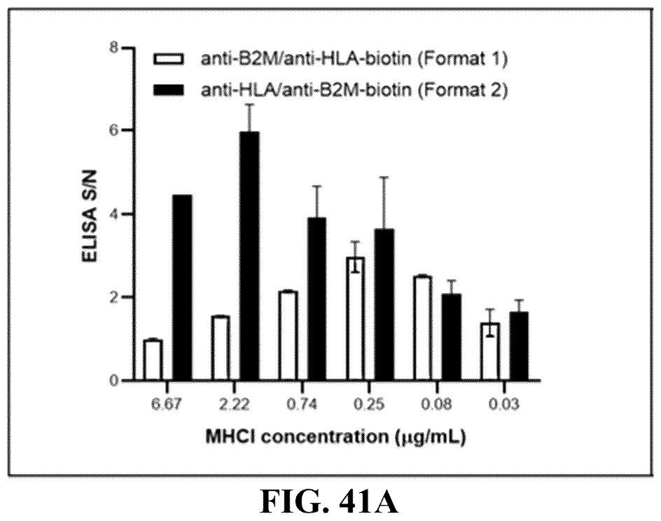

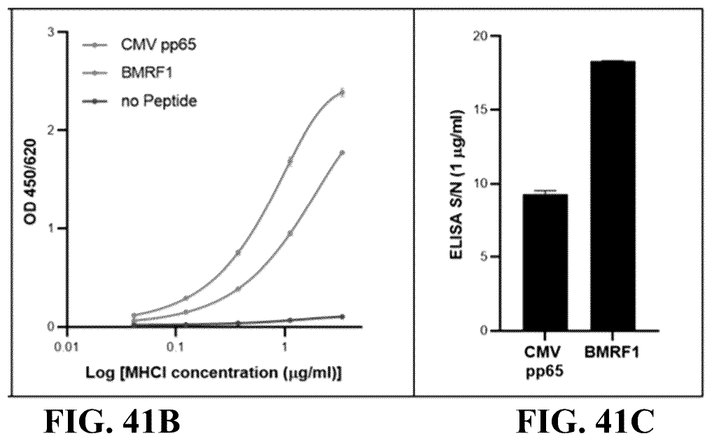

One of the main objectives of this manuscript was to develop a robust HTP workflow for the identification of peptides containing a non-natural UV cleavable amino acid (conditional MHCI ligand) that could form a stable conditional MHCI complex. The first step in this process was to develop an ELISA assay that could measure the formation of a stable MHCI complex after a refold screen. Since the HLA components are not biotinylated at this stage, we could not use the widely published streptavidin-based ELISA. We instead evaluated two formats: 1) capture with anti-B2M and detection with anti-HLA (clone W6/32) and 2) capture with anti-HLA (clone W6/32) and detection with anti-B2M. In both cases, the detection antibody was labeled with biotin and signal generation was induced after the addition of streptavidin-HRP and substrate. CMV pp65 peptide and HLA-A*02:01 were used for these initial screens. Although the signal and detection range was comparable for both formats, the normalized values were much higher for format 2) (FIG. 41A black bars) than format 1) (FIG. 41A, white bars) at concentrations above 0.25 μg/mL. At these higher concentrations, there was no or minimal difference in ELISA signal between the peptide and no peptide control. The anti-HLA used in these assays recognizes a conformational epitope on MHCI and should be selective for properly folded MHCI. In contrast, anti-B2M capture is not dependent on properly folded MHCI and will capture complexes with properly folded HLA as well as partially denatured HLA. Therefore, the decreased signal to noise for ELISA format 1) at higher concentrations is likely due to the capture of partially folded MHCI. FIG. 41B shows the ELISA results as a function of MHCI concentration under optimized assay conditions for ELISA format 2) using MHCI molecules refolded with CMV pp65 peptide, BMRF1 peptide and no peptide. As the concentration was increased, we observed an increase in the OD450/620 ELISA signal for both the CMV pp65 and BMRF1 antigens but little to no increase in signal for the no peptide control, which is consistent with the detection of properly refolded MHCI complex. FIG. 41C shows the ELISA signals normalized to the no peptide control at a MHCI concentration of 1 μg/mL for both CMV pp65 and BMRF1. Both antigens had a signal that was 10-fold greater than background, demonstrating that this assay format produces highly sensitive signal to noise and can be readily used to identify antigens that can form a stable MHCI complex during the refold step.

Identification of Peptide Binders and Conditional MHCI Ligands Across HLA Alleles.

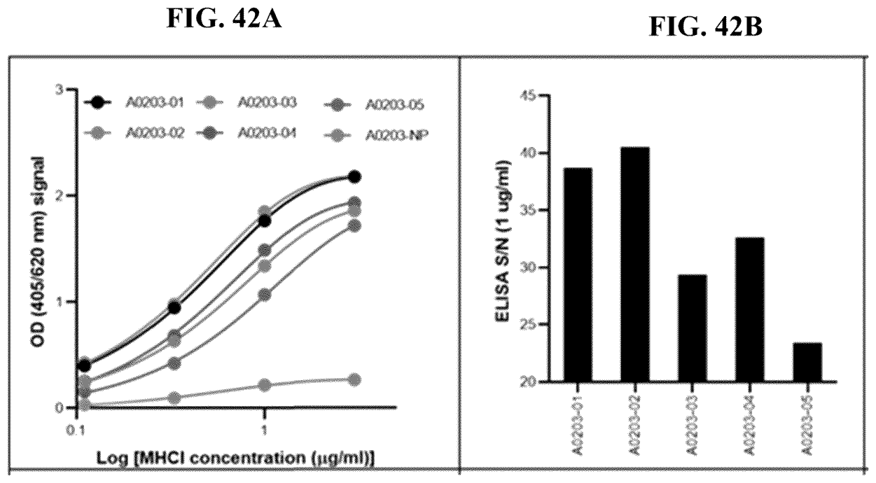

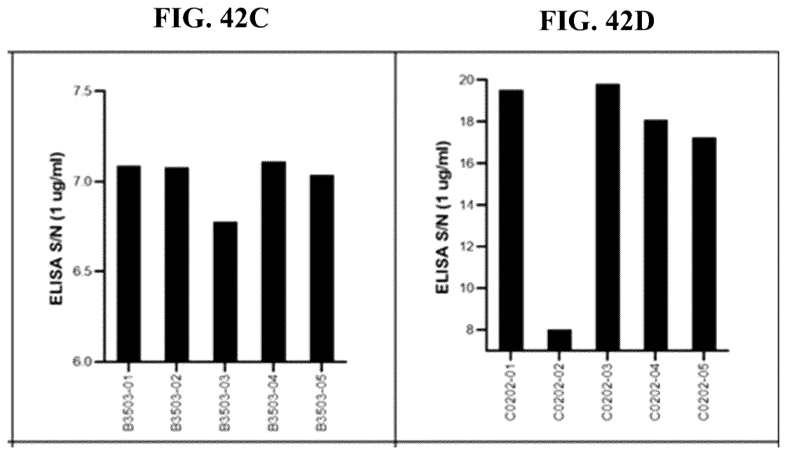

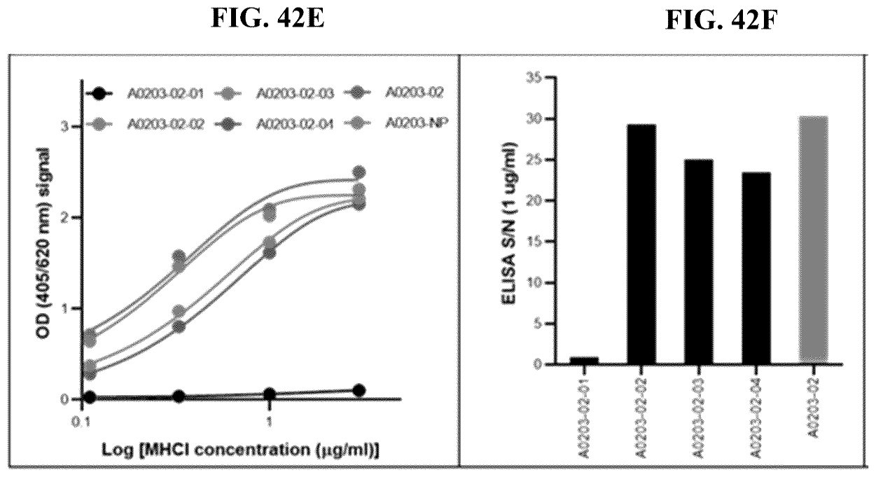

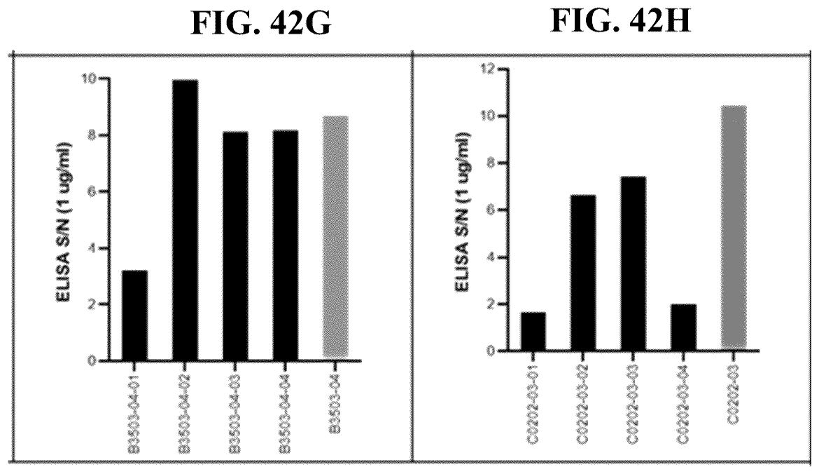

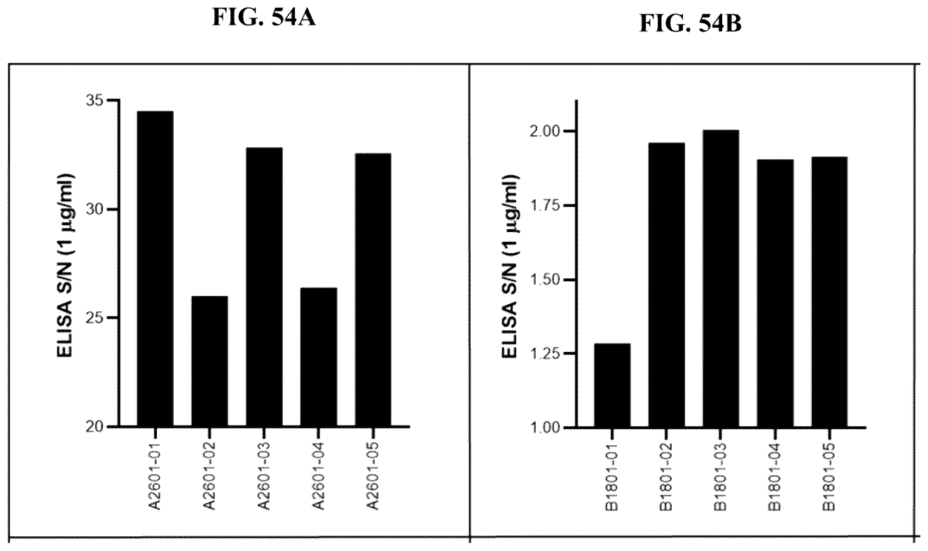

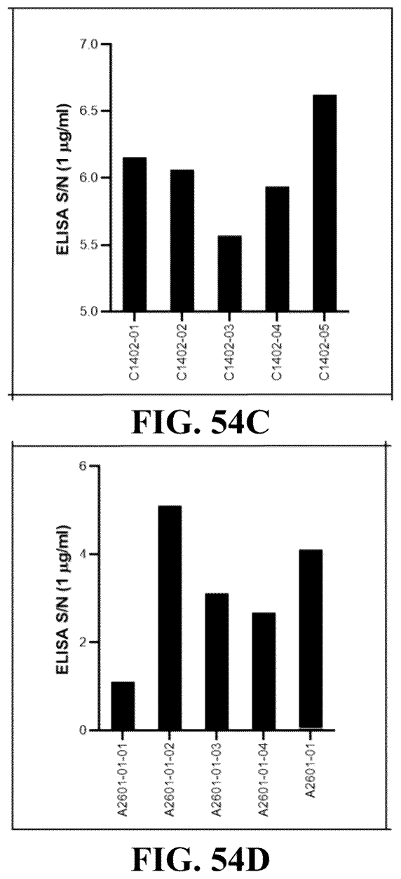

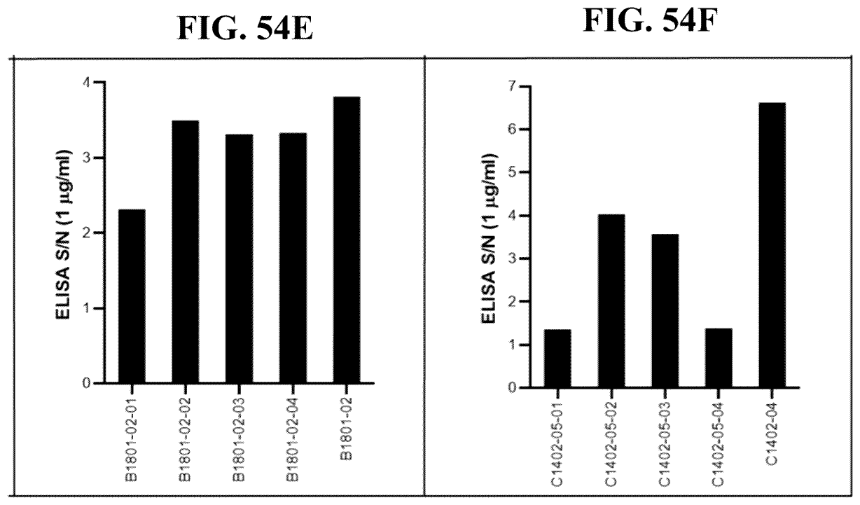

Peptides were selected from IEDB (Table 3). The ability of these peptides to form a stable MHCI complex with the corresponding HLA allele was analyzed using the ELISA assay described above. Titration curves for the refolded MHCI complexes generated with the five peptides and HLA-A*02:03 are shown in FIG. 42A. The assay was performed at MHCI concentrations ranging from 0.1 to 3.0 μg/mL and, as observed for the positive controls in FIG. 41B, there was an increase in the ELISA OD at increasing MHCI concentrations and the signal began to saturate at 1 μg/mL. In addition, we observed only a minimal increase in signal for the negative control across the titration range. Because the signal appeared to saturate at 1.0 μg/mL, we selected this concentration to show the normalized ELISA values for the A*02:03, B*35:01 and C*0202 alleles (FIGS. 42B-42D). The signal to background for the five peptides screened against A*02:03 was relatively high, with values ranging between 20 to 40. This suggests that all peptides selected from IEDB were not only binders, but could also form stable MHCI complexes upon refolding. The A0203-02 peptide yielded the highest normalized OD value and was selected for the design of UV peptides. For B*35:03, the normalized OD from the selected peptides also had a relatively high signal to background, ranging from 6.75-7.25 (FIG. 42C). B3503-04 yielded the highest OD value and was subsequently selected to design candidate conditional MHCI ligands. The normalized ODs for the C*02:02 peptides were all between 18 and 20 except C0202-02, which was much lower (˜8) (FIG. 42D). C0202-03 yielded the highest normalized OD value and was selected to design candidate conditional MHCI ligands. A*26:01, B*18:01, and C*14:02 were also tested and similar results were observed (FIGS. 54A-54F). Although the signal to noise for all alleles tested was relatively high and provided confidence that the assay was identifying peptides that formed a stable complex, there were significant differences in the magnitude of the normalized signal across the alleles. We believe these results are likely due to the variability in the affinity of the pan-HLA antibody for the different HLA alleles. In contrast, the variability within a given allele (e.g. A*02:03 and C*02:02) was likely due to difference in peptide affinity across the different peptides screened.

These selected peptides were then used to design peptides containing the non-natural UV cleavable amino acid (conditional MHCI ligands). Variants of the top-performing peptides in the initial screen (i.e. A0203-02, B3503-04, and C0202-03) (FIGS. 42B-42D) were designed with a UV-cleavable amino acid (denoted J) substituted at positions 2, 4, 6, and 8 from the N-terminus to identify UV-peptides that form stable complexes across the different HLA alleles. A titration curve of the four conditional MHCI ligands screened derived from the A0203-02 peptide are shown in FIG. 42E. As was observed for the non-UV cleavable peptides, an increase in the ELISA OD was observed with increasing MHCI concentration and the values started to saturate at 1 μg/mL (FIG. 42E). The normalized ELISA signal for all conditional MHCI ligands across the different alleles is shown in FIGS. 42F-42H. Interestingly, all conditional MHCI ligand variants with J amino acid substitution at position 2 showed very low normalized ELISA values (FIGS. 42F-42H), indicating minimal or no MHC complex formation (A0203-02-01, B3503-05-01, C0202-03-01 and C0202-03-04) relative to the parent peptide (gray bar FIGS. 42F-42H). This finding was expected since this position is known to be a MHCI-peptide anchor position. All other conditional MHCI ligands screened for A0203-02 and B3503-04 yielded normalized OD readout similar to the parent peptide (FIGS. 42F-42G). In contrast, all conditional MHCI ligands for C0202-02 had lower OD values when compared to the parent; however, the normalized OD for C0202-02-02 and C0202-02-03 were still relatively high at 6 and 8, respectively (FIG. 42H), indicating that the UV cleavable amino acid had a slightly negative impact on MHCI stability. The conditional MHCI ligands resulting in the highest normalized ELISA OD value (A0302-02-02, B3503-05-02 and C0202-03-03) were then selected for scaled-up production. A similar analysis was performed to identify optimal conditional MHCI ligands for A*26:01, B*18:01, and C*14:02 alleles (FIGS. 54A-54F).

Scaled-Up Refold and Purification of MHCI Monomers.

Generation of recombinant MHCI complexes through refolding of denatured B2M and HLA purified from E. coli inclusion bodies in the presence of the peptide of interest was initially developed over 20 years ago. Since this initial report, there have been many studies and methods developed for generation and purification of MHCI complexes using a refold protocol. The vast majority of these methods include some variation of the following process: 1) mix HLA and B2M with peptide in generic refold buffer with redox/oxidation agents (incubation time may vary from 1-5 days), 2) filter refolded material to remove aggregates, 3) concentrate down to volume compatible with size exclusion chromatography (SEC) (1-2 mL depending on the column), 4) purify using SEC, 5) biotinylate purified refolded MHCI complex and 6) purification of MHCI complex from biotinylation reaction (SEC, spin column filters, etc.) The majority of these production methods are only practical at a 1 L scale or smaller and yield only a few milligrams of refolded material; therefore, multiple production runs would be required to produce sufficient material for use in supporting a clinical program. Multiple runs could potentially result in lot-to-lot variability, which may confound interpretation of downstream tetramer staining data. To address these limitations, we developed a novel workflow to enable MHCI refold and purification to be scaled up to 15 liters.

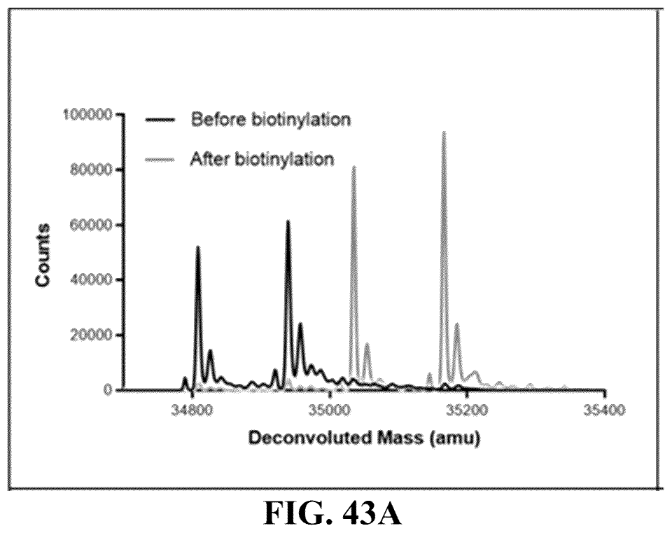

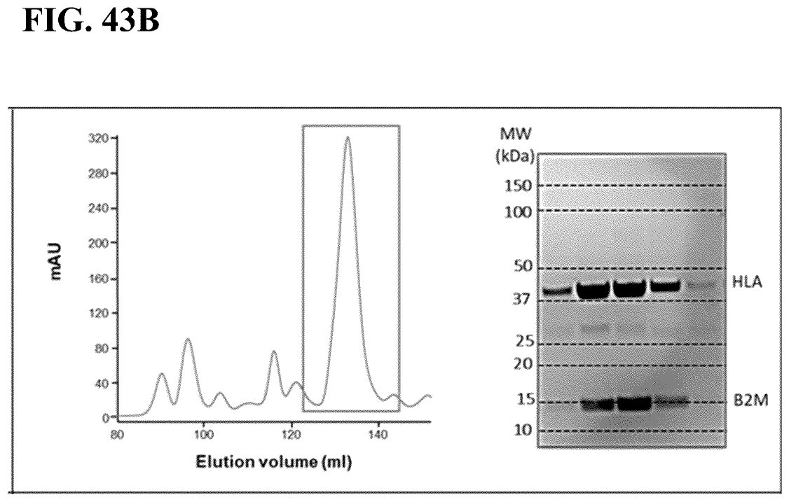

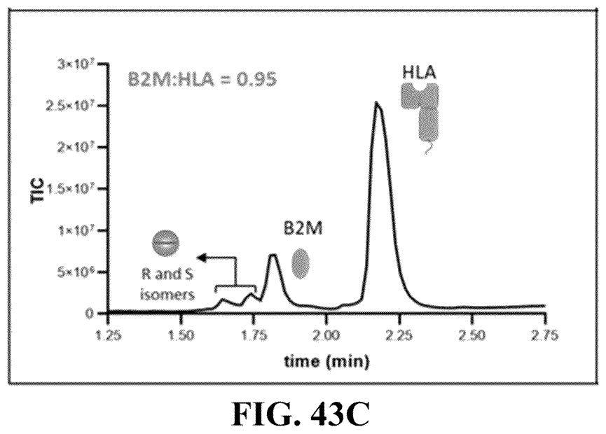

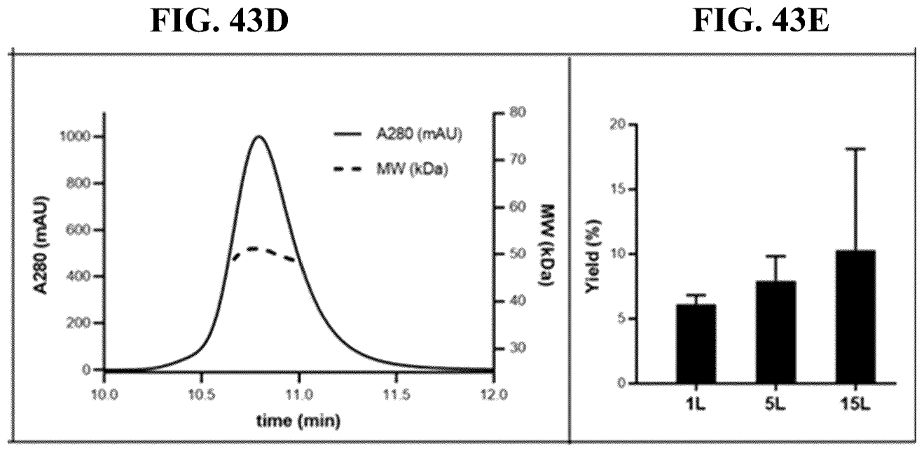

For method optimization, the HLA-A*02:01 specific published conditional MHCI ligand were used. The primary limitation to scaled-up production is the need for an SEC purification step, which requires concentrating samples to less than 1 mL. In addition, the biotinylation step is typically performed on purified refolded MHCI complexes and requires a secondary purification step, which further limits scaled-up production. To address these limitations, we developed a 3-step production process that includes the refold reaction, in-process biotinylation, and anion exchange chromatography purification. LC/MS analysis of the HLA component of the MHCI complex before and after the biotinylation step is shown in FIG. 43A. The black line shows the HLA protein before biotinylation. Two peaks were observed at 34812 Da and 34943 Da, which correspond to the HLA protein with and without the N-terminal methionine group. These two populations are likely caused by incomplete modification and subsequent removal of formylmethionine and the N-terminal methionine by formylmethionine deformylase and methionine aminopeptidase (MAP), respectively, which can vary depending on the adjacent amino acids and in some cases the N-terminal methionine is not removed. Therefore, it is likely that the N-terminal HLA sequence was not ideal for total MAP activity, so only partial N-terminus removal was observed. After biotinylation, we observed an increase in the mass for both peaks of ˜244, which corresponds to the mass of biotin (FIG. 43A). No residual peaks were observed at the non-biotinylated masses, indicating 100% biotinylation. These combined results demonstrate the feasibility of adopting an in-process biotinylation step to eliminate the need for two purification steps.

After completion of the biotinylation step, the resulting biotinylation reaction was buffer exchanged into 25 mM Tris and prepared for purification via anion exchange chromatography. Anion exchange was selected for purification over SEC because it is amenable to direct loading of the large volumes used during the biotinylation step (10-100 mL). A representative Q-HP anion chromatogram of a 1 L refold is shown in FIG. 43B. A large peak was observed at an elution volume of ˜130 mL with several smaller peaks, likely representing minor contaminants from the inclusion body purification. Fractions from the major peak were run on SDS-PAGE, and bands corresponding to the expected MW for HLA-A*02:01 and B2M were observed across the peak (FIG. 43B). Fractions were pooled based on band intensity in the SDS-PAGE analysis and run on LC/MS and SEC-MALS. The TIC chromatogram of the purified biotinylated MHCI complex is shown in FIG. 43C. The two adjacent peaks at a retention times of 1.7 and 1.75 min corresponded to the R and S diastereomers of the conditional MHCI ligand resulting from the use of a racemic UV amino acid. The peaks at retention times of 1.8 min and 2.2 min correspond to B2M and HLA-A*02:01, respectively. Standard curves of both B2M and HLA-A*02:01 were generated and the area under the curve was used to quantify the molar concentration and molar ratios of B2M to HLA. If the refold process resulted in the proper pairing of B2M to HLA, the molar ratios of the two components should be close to 1. For this preparation, the ratio was calculated to be 0.95, suggesting proper pairing. The MHCI complex was further analyzed by SEC-MALS for native mass analysis to further confirm proper 1:1 HLA:B2M pairing. The A280 SEC chromatogram peak was highly symmetric, indicative of a homogeneous protein sample, and no aggregate peak was observed (FIG. 43D). The MW across the MHCI peak ranged from 48.8 to 51.3 kDa (FIG. 43D, red dashed line) and the average value was 49.1 kDa, close to the expected MW of the MHCI complex (48.1 kDa). The collective LC/MS and SEC-MALS analyses suggest that the refold and purification protocol yielded a highly pure and properly folded MHCI complex.

One of the primary goals of developing a novel purification workflow was to enable scaled-up production. To test scalability, the optimized 1 L protocol was performed at the 5 L and 15 L scales. The yields at these production scales are shown in FIG. 43E. Interestingly, we observed a gradual increase in yield as the process was scaled up from 1 L (˜6%) to 5 L (˜8%) to 15 L (˜10%), although this increase was not statically significant. The amount of MHCI complex generated from the 1 L scale and 15 L scale was −2.4 mg and −60 mg, respectively, which corresponds to a 25-fold increase in material generation per refold at the 15 L scale. These combined findings demonstrate the feasibility of scaling up the production and purification of MHCI complexes using the workflow described in this study.

Scaled-Up Production and Peptide Exchange Analysis of Six MHCI Monomers with UV Peptides Identified from the HTP Screen.

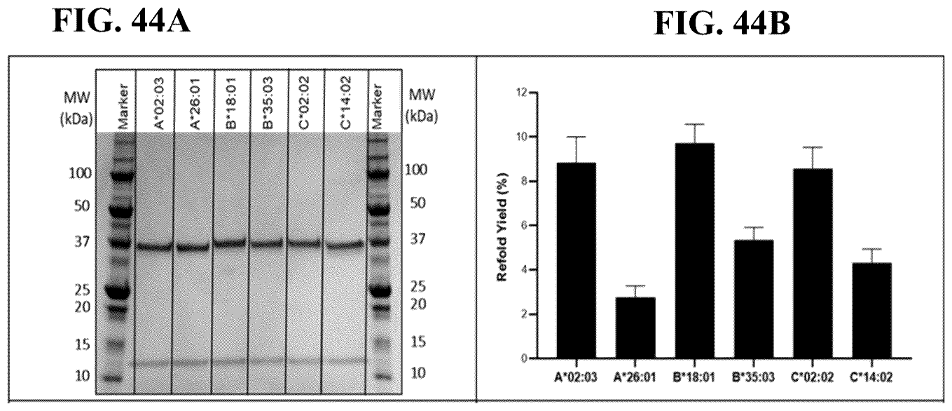

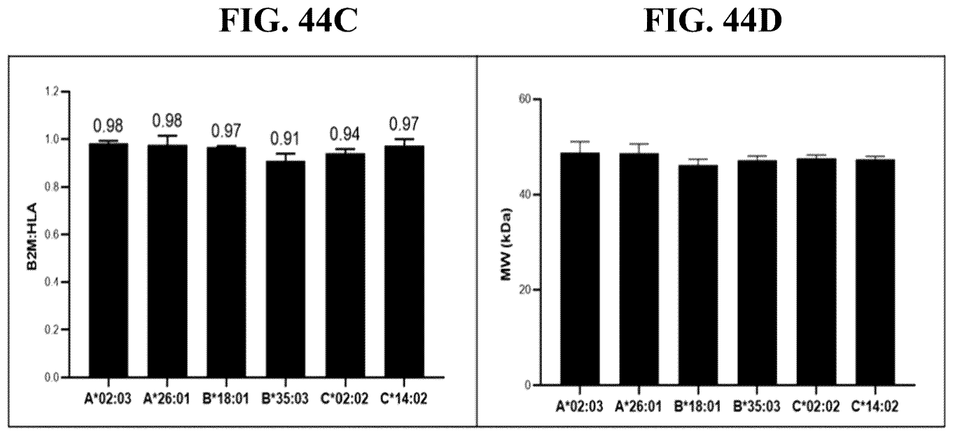

The refold and purification protocol described above was applied to the large-scale production of MHCI complexes with the conditional MHCI ligands identified in the HTP screen. The Q-HP anion chromatograms of all six constructs and corresponding SDS-PAGE of pooled fractions are shown in FIGS. 54A-54F. The chromatograms for these refolds are very similar to HLA-A*02:01 (FIG. 43B), with clear HLA and B2M bands in the SDS-PAGE. The pooled fractions for each HLA allele were run on SDS-PAGE and bands corresponding to HLA and B2M were observed with high purity (FIG. 44A). The yields from the 1 L refolds varied from sample to sample, where A*02:03, B*18:01 and C*02:02 had the highest yields, ranging from 8-11%, followed by B*35:03 (˜5%), C*14:02 (˜4%) and A*26:01 (˜2.5%). This variability is likely due to differences in amino acid sequence content and susceptibility to aggregation during refold as well as the ability of the peptide to form a stable complex. Although there was variability, the lowest yield of 2.5% still produced 1 mg of material from a 1 liter refold and this could scale to >15 mg at the 15 liter scale, which is sufficient to cover >30,000 tetramer stains.

LC/MS and SEC-MALS analyses were performed to further evaluate the quality of these MHCI monomers and assess whether the B2M and HLA were properly paired. The results of the LC/MS analysis of the B2M:HLA ratio across the six alleles is shown in FIG. 44C. As was observed for the HLA-A*02:01 MHCI complex, the B2M:HLA ratios for all these samples were close to 1. The MHCI complex was further analyzed by SEC-MALS for intact native state mass analysis. The average MW across the MHCI peak for all six MHCI complexes analyzed is shown in FIG. 44D. The MW for all six constructs ranged from 47.4 to 48.8 kDa, which is well within the expected mass range of these complexes (47.5-48 kDa). These combined findings demonstrate that not only was the refold protocol and purification workflow broadly applicable, but also that the conditional MHCI ligands identified in the small-scale HTP assay were able to form stable MHCI complexes upon scale-up.

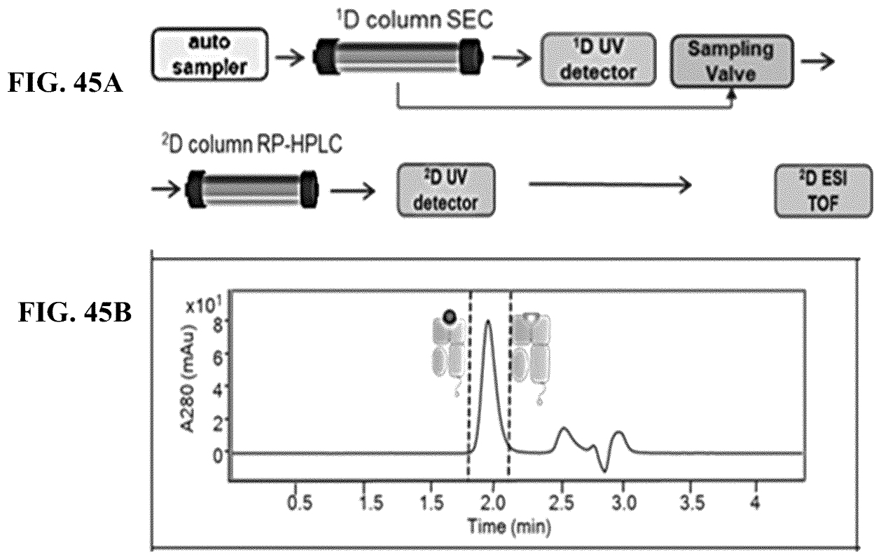

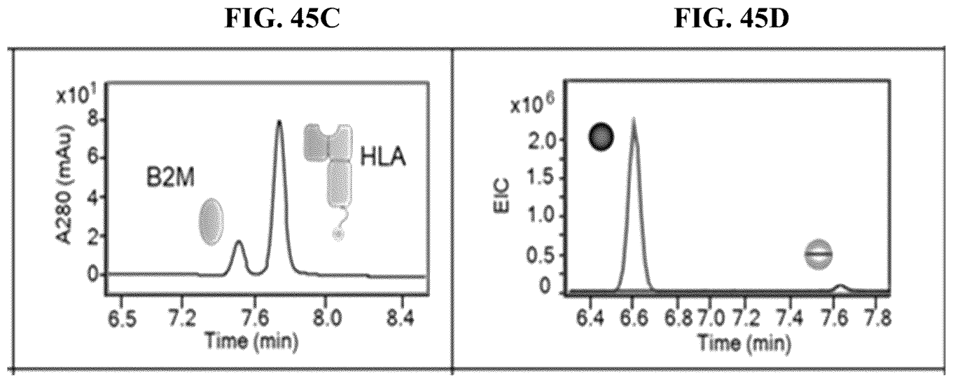

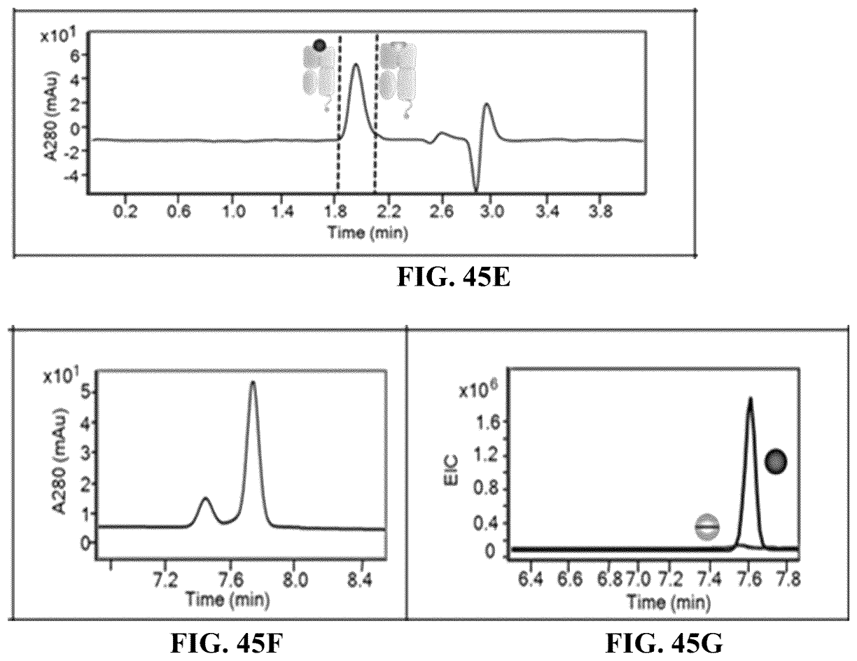

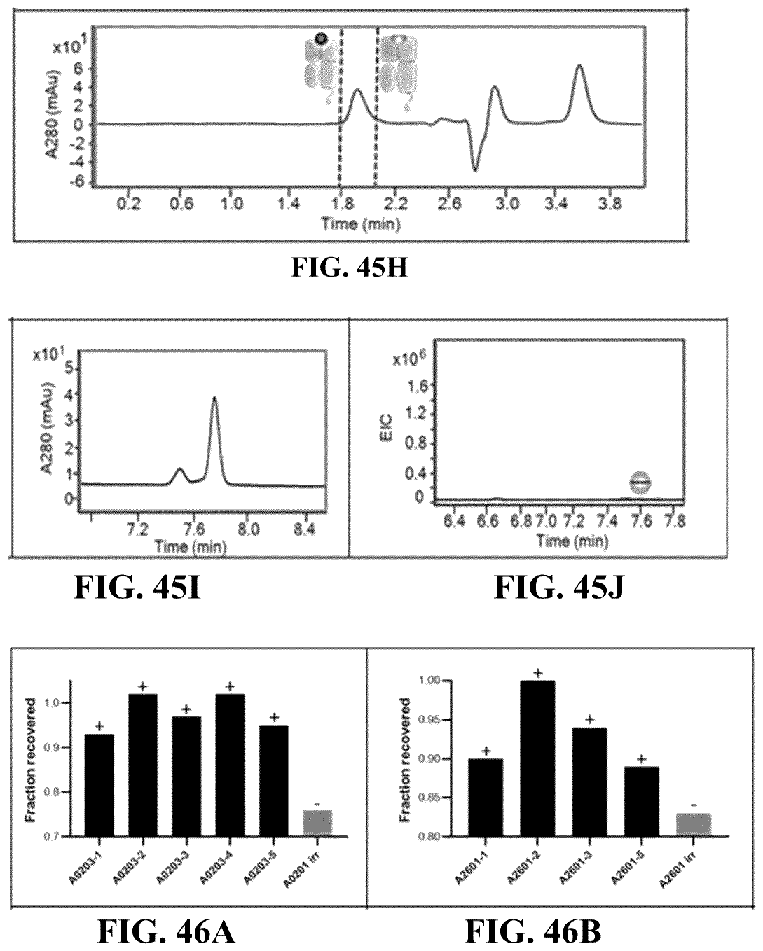

In addition to identifying novel conditional MHCI ligands that could be used for scaled-up production of MHCI monomers, it was desirable to demonstrate that these complexes could undergo peptide exchange upon UV exposure to enable HTP generation of MHCI complexes. One of the most widely used methods to measure peptide exchange of the conditional MHCI ligands after UV exposure is ELISA. Although this assay has proven valuable in demonstrating peptide exchange upon UV exposure, the assay is semi-quantitative and does not provide a direct measurement of the exchanged peptide, nor does it allow for quantification of the cleaved peptide. To address these limitations, we developed a 2D LC-MS analysis method for direct quantification of the peptide loaded into the complex during the exchange process. A schematic of the assay is shown in FIG. 45A. The first step (1^{st} dimension) in this method is injection of the peptide exchange reaction mixture (MHCI complex after exposure to UV+100-fold molar excess of exchanged peptide) on an analytical SEC column, which enables collection of the MHCI complex by a sampling valve without excess peptide. The second step (2^{nd} dimension) is injection of the material collected from the MHCI peak onto RP-HPLC. The organic phase of the RP-HPLC step results in dissociation and denaturation of the HLA, B2M and peptide contained within the complex, which enables analysis and quantitation of the individual components of the MHCI complex by A280 and LC/MS. An example of the 1^{st} dimension SEC chromatogram of peptide exchange for the control HLA-A*02:01 conditional MHCI ligand with the CMV pp65 epitope is shown in FIG. 45B. The chromatogram shows one dominant peak corresponding to the MHCI complex. A fluctuation in the chromatogram A280 signal was consistently observed between 2.5 and 3 min, which corresponds to the opening and closing of the sampling valve. Since there is a large pressure difference between the 1^{st} and 2^{nd} dimension, this fluctuation is likely associated with the sudden change in pressure as the valve opens and closes. An example of the A280 chromatogram for the 2^{nd} dimension HPLC step is shown in FIG. 45C. The HLA and B2M peaks are clearly visible but no A280 peak for the CMV pp65 peptide or conditional MHCI ligand are observed because they do not contain any tryptophan or tyrosine residues and no inherent A280 absorbance. To analyze the peptide composition, the extracted ion exchange chromatograms for the exchange peptide and uncleaved conditional MHCI ligand were generated (FIG. 45D). As expected, we observed a large peak corresponding to the CMV pp65 peptide, indicating successful peptide exchange. However, very low levels of the intact conditional MHCI ligand were also observed. This result suggests that either the purified peptide has a synthesis-derived contaminant that is carried through with a similar mass or a small fraction of the conditional MHCI ligand is protected from UV cleavage when they are in the complex. This peak is also observed when the peptide alone is cleaved in the absence of MHCI, suggesting that this is a contaminant. Regardless, the relative fraction of intact conditional MHCI ligand was very low (˜1%) and should have minimal impact on downstream tetramer staining. The same analysis was performed to verify the presence of cleaved conditional MHCI ligand, and no peaks were detected. By adopting a 2D LC/MS assay to analyze peptide exchange, we were able to measure how the peptide content in the MHCI complex changes during the peptide exchange process. We believe this is a powerful tool that can be used to better understand the parameters relevant to peptide exchange, which cannot be quantified using other traditional methods such as ELISA.

This method was then used to assess peptide exchange with the MHCI complexes generated using the scaled-up production workflow with the novel conditional MHCI ligands identified in the HTP small-scale assay. For each MHCI complex, four—five peptides that were found to be binders in the small-scale HTP screen (FIGS. 42B-D and 54A-54C) and one known non-binding peptide were used as positive controls and a negative control, respectively. The 1^{st} dimension SEC chromatogram for HLA-A*02:03 with peptide A0203-05 showed a clear single peak (FIG. 45E). The 2^{nd} dimension HPLC A280 chromatogram of the peptide exchanged complex also shows the expected B2M and HLA peaks (FIG. 45F). In the EIC analysis of the 2^{nd} dimension, a large peak corresponding to the extracted mass of the A0203-05 peptide was observed (black line FIG. 45G). However, similar to A*02:01 and CMV pp65 peptide exchange, a small peak corresponding to the intact conditional MHCI ligands was also observed (dashed line FIG. 45G). This phenomenon was seen for all conditional MHCI ligands tested and indicates there is some low level contaminants that are present and carried through the peptide exchange process. It is worth noting that in all cases the total fraction of contaminant peptide was less than 1%. The 1^{st} dimension SEC chromatogram of the HLA-A*02:03 MHCI complex after peptide exchange with a known non-binder is shown in FIG. 45H. The overall peak area under these conditions was lower than when the peptide exchange process was performed with a peptide binder (FIGS. 41E vs 41H). In addition, the A280 HPLC chromatogram in the 2^{nd} dimension contained two peaks but the overall peak areas were much lower than when a peptide binder was used (FIGS. 45F vs 45I). These combined data suggest that there was significant material loss when the peptide exchange was performed with a non-binder, likely due to protein aggregation. Interestingly, a peak at the end of the SEC run (FIG. 45H, retention time −3.6 min) was consistently observed for the negative control exchange samples across all alleles tested, which we believe is due to partially denatured HLA interacting with the column. Finally, in the EIC analysis, no peak corresponding to the A*02:03 non-binder peptide was observed indicating that peptide exchange did not occur (FIG. 45J). It was surprising that even though no peptide was associated with the complex, a relatively prominent A280 peak was observed in the 1^{st} dimension, although much smaller than the peak observed with peptide exchange. These results suggest that some of the complex remains associated in the absence of a peptide but it is likely of low quality and not properly folded.

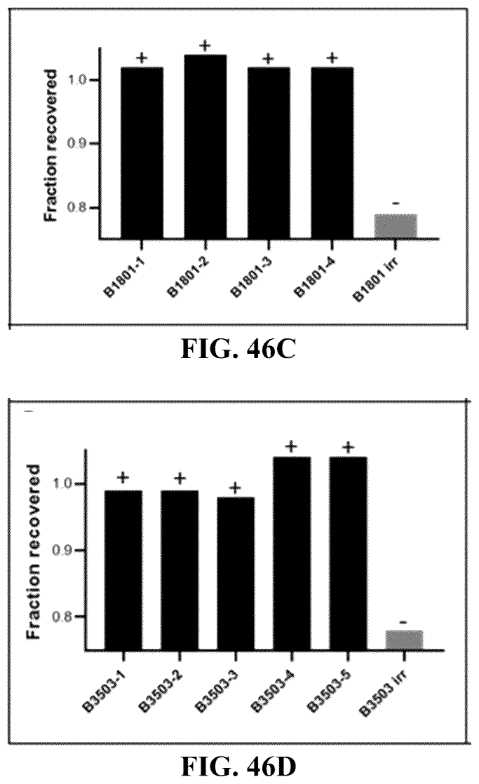

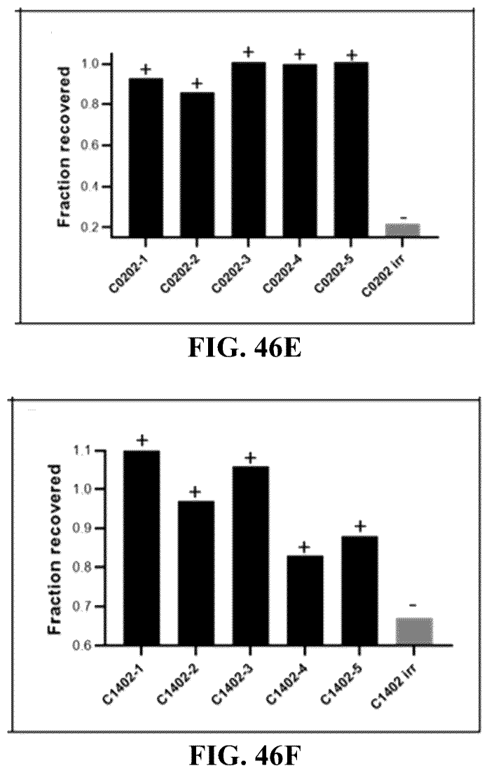

Quantification of the 1^{st} and 2^{nd} dimension data is shown in FIGS. 46A-46F. For this analysis, the fraction of the A280 MHCI peak that was recovered after exchange in the Pt dimension (SEC) and the presence of the exchanged peptide in the 2^{nd} dimension (EIC) were evaluated. FIGS. 46A-46F show the fraction of A280 MHCI peak recovered (normalized to non-peptide exchange control) for the different MHCI complexes across the four—five positive controls and one negative control. The symbol above the graph indicates if the peptide was detected in the 2^{nd} dimension (+=detected, −=not detected). For HLA-A*02:03 (FIG. 46A), the fraction recovered for the positive control binder peptides varied from 0.9 to 1 and the exchange peptide was observed in the 2^{nd} dimension for all peptides. As described above, the fraction recovered for the negative control was still relatively high (˜0.76) even though no exchanged peptide, cleaved conditional MHCI ligand and very low levels of intact conditional MHCI ligands (<1%) were observed. This suggests that the complex remains somewhat intact for a period of time in the absence of a peptide, although it is likely not properly folded given the instability of the HLA allele in the absence of a peptide. It is also worth noting that because the difference in fraction recovered between the positive and negative control was only 26%, it is possible this would have been missed by ELISA given this assay only measures pairing between HLA and B2M and not peptide content.

Similar results were observed for A*26:01 (FIG. 46B), B*18:01 (FIG. 46C), B*35:03 (FIG. 46D) and C*14:02 (FIG. 46F). In all cases, the difference in peak area between the positive and negative control in the first dimension was less than 20-25%. In contrast, there was a drastic difference in the negative and positive controls for C*02:02 (FIG. 46E). For this sample, ˜80% of the material degraded when a known non-binder peptide was included in the peptide exchange. Although the exact reason for this is not known, it is likely due to the ability of the different alleles to remain associated with B2M when a peptide has been removed from the peptide groove on HLA. Regardless, these combined results clearly indicate that conditional MHCI ligands that allow for scaled-up production of the MHCI peptides and subsequent peptide exchange for HTP generation of MHCI monomers and tetramers were identified.

In this study, a workflow was developed that enabled the identification and validation of new conditional MHCI ligands that form stable UV peptide MHC complexes. This workflow included screening known peptide binders identified in the IEDB to form stable refolded MHCI complexes. Peptides containing a non-natural UV cleavable amino acid were designed based on the top performing peptide in the initial screen. The UV peptides were then screened in the same ELISA assay and the top performers were selected for scaled-up production. A novel MHCI complex purification and biotinylation protocol was developed using the published conditional MHCI ligand for HLA-A02:01 to enable scale up production beyond traditional scales (e.g. 1 L). In addition, next-generation analytical techniques (LC/MS, 2D LC/MS and SEC-MALS) were used to confirm the quality of complexes generated. The optimized refold production and purification protocol and next generation analytical techniques were applied to the conditional MHCI ligands identified in the ELISA screen. This analysis demonstrated that the new conditional MHCI complexes were purified to high purity, properly refolded, and of high quality. Finally, we evaluated peptide exchange for the conditional MHCI complexes after UV exposure with validated peptide binders using 2D LC/MS. All conditional MHCI complexes were able to undergo peptide exchange upon UV exposure. These combined results demonstrate that a workflow was developed that can be used to identify conditional MHCI ligands for new HLA alleles. This approach has the potentially to be broadly applied and to enable HTP generation of MHCI monomers and tetramers across a broader range of HLA alleles, which could be critical to enabling the use of MHCI tetramers to monitor neoantigen specific T-cells in the clinic.

### Example 10: High-Throughput Identification of Conditional MHCI Ligands and Scaled-Up Production of Conditional MHCI Complexes

Despite the need to monitor the impact of Cancer Immunotherapy (CI)/Immuno-Oncology (10) therapeutics on neoantigen-specific T-cell responses, very few clinical programs incorporate this aspect of immune monitoring due to the challenges in high-throughput (HTP) generation of MHCI tetramers across a wide range of HLA alleles. This limitation was recently addressed through the development of MHCI complexes with peptides containing a non-natural UV cleavable amino acid (conditional MHCI ligands) that enabled HTP peptide exchange upon UV exposure. Despite this advancement, the number of alleles with known conditional MHCI ligands is limited. We developed a novel workflow to enable identification and validation of conditional MHCI ligands across a range of HLA alleles. First, known peptide binders were screened via an enzyme-linked immunosorbent assay (ELISA) assay. Conditional MHCI ligands were designed using the highest-performing peptides and evaluated in the same ELISA assay. The top performers were then selected for scale-up production. Next-generation analytical techniques (LC/MS, SEC-MALS, and 2D LC/MS) were used to characterize the complex after refolding with the conditional MHCI ligands. Finally, we used 2D LC/MS to evaluate peptide exchange with these scaled-up conditional MHCI complexes after UV exposure with validated peptide binders. Successful peptide exchange was observed for all conditional MHCI ligands upon UV exposure, validating our screening approach. This approach has the potential to be broadly applied and enable HTP generation of MHCI monomers and tetramers across a wider range of HLA alleles, which could be critical to enabling the use of MHCI tetramers to monitor neoantigen-specific T-cells in the clinic.

The most common methods for tracking T-cell responses are ELISPOT and MHC tetramer staining. The ELISPOT assay is a functional assay that measures cytokine release from T-cells upon stimulation of PBMCs with antigens. The benefits of this assay are that it is allele- and neoepitope-independent (i.e., only the neoantigen needs to be known) and it is a functional readout. The drawbacks to the assay are that it is semi-quantitative and there is no way to assess T-cell phenotype, which could be critical to understanding factors important to generating a protective immune response. MHCI tetramer-based detection utilizes recombinant MHCI monomers multimerized into tetramers via streptavidin conjugation as neoantigen-specific T-cell staining reagents. This method allows for staining of multiple specificities as well as phenotype markers. MHCI tetramers also allow for quantitative analysis of the exact number of neoantigen-specific T-cells and how this changes during the course of treatment. In many respects, MHCI tetramer-based detection can therefore provide a more detailed understanding of the effect of treatment on the neoantigen-specific CD8+ T-cell response.

Despite the advantages of MHCI tetramer detection, this approach has not been widely adopted as a bio-marker strategy across clinical programs because of challenges associated with generating reagents. MHCI tetramer generation requires a time-consuming, multi-day, low-yield refold process including multiple chromatography steps. Furthermore, each human has six different HLA alleles and the HLA allele is highly polymorphic (nearly 20,000 HLA class I alleles exist). Additionally, not only is the neoantigen profile unique to each patient but also, 10-100s of patient-specific MHCI tetramers would be required to gain a complete picture of the T-cell landscape in a given patient. Therefore, MHCI tetramer-based detection of neoantigen specific T-cell responses would require implementation of a personalized MHCI tetramer platform, which is not possible using traditional MHCI generation protocols.

Several novel methods of preparing MHCI reagents have been developed to address these limitations. One approach is to engineer stabilizing disulfides in the HLA allele to enable the formation of stable MHCI complexes in the presence of a dipeptide. These disulfide-stabilized MHCI reagents have been referred to as “empty” MHCI complexes and can be loaded with a peptide or epitope by simply adding the peptide of interest to the empty MHCI complex. This strategy has been demonstrated for both murine and human (A*02:01) MHCI complexes, and comparable tetramer staining results were reported between MHCI reagents produced using this approach and traditional refold approaches. Another method uses an allele-specific UV-cleavable peptide, also called a conditional MHCI ligand, to form an MHCI complex, in which the pep tide binds with high affinity when intact and low affinity when cleaved. This functionality enables peptide exchange upon UV exposure when MHCI complexes that have been assembled with conditional MHCI ligands, called conditional MHCI complexes, are incubated in the presence of a high-affinity peptide binder of interest. Conditional MHCI complexes for a given HLA allele can be refolded at large scale, and the end user can then exchange the conditional MHCI ligand for any other peptide of interest.

Both of these methods provided a breakthrough in the quest to enable the use of personalized MHCI tetramers to monitor neoantigen specific T-cells in the clinic. One of the major drawbacks of the “empty” MHCI is the need to identify novel engineered disulfides that stabilize the “empty” complex and dipeptides that enable the “empty” complex to refold for each different HLA allele. Similarly, one of the drawbacks of the conditional MHCI approach is the need to identify and design specific peptides for each HLA allele. However, given that high-throughput (HTP) screening of peptide binders is easier from a resource standpoint than generating multiple engineered constructs in combination with screening dipeptide stabilizers needed for “empty” MHCI, the goal of this manuscript was to further expand the repertoire of conditional MHCI ligands. To the best of our knowledge, conditional MHCI ligands have only been published for 24 HLA alleles. Although these alleles are some of the most prevalent, neoantigen coverage across a broad cohort of diverse patients will still be minimal. Therefore, there is a need to develop workflows to enable the expansion of allele coverage.

In addition, analytically validating MHCI complexes after refolding or peptide exchange has utilized a limited number of analytical techniques including enzyme-linked immunosorbent assay (ELISA) assays and gel electrophoresis. Although these techniques have proven useful to determine if the MHCI complex is present and for semi-quantitative analysis of affinity and stability, several other important parameters, such as HLA:B2M ratio, aggregation, and oxidation state are not captured. Several protein analytical tools exist to evaluate these parameters, including liquid chromatography/mass spectrometry (LC/MS), 2D LC/MS and size-exclusion chromatography/multi-angle light scattering detection (SEC-MALS); yet, these tools have rarely been used to characterize MHCI complexes after refold or peptide exchange.

Here is an experimental workflow that allows for the identification and validation of new combinations of conditional MHCI ligands and HLA alleles that form stable conditional MHCI complexes. We developed and validated an ELISA assay for detection of stable conditional MHCI complexes. We initially screened five published peptide binders reported in the Immune Epitope Database and Analysis (IEDB) across six HLA alleles (A*02:03, A*26:01, B*18:01, B*35:03, C*02:02, C*14:02) and designed conditional MHCI ligands based on the top binders. The conditional MHCI ligands were then screened in the ELISA assay, and the top performers were selected for scale-up production. For MHCI production, a novel MHCI purification and biotinylation protocol was developed, and next-generation analytical techniques were used to confirm the quality of generated complexes. These methods were further applied to characterize the conditional MHCI complexes generated with the newly identified conditional MHCI ligands. Finally, we verified peptide exchange with validated peptide binders after UV exposure using 2D LC/MS. In summary, we developed a validated workflow to identify conditional UV peptides for new HLA alleles that can be broadly applied in order to greatly expand HLA allele coverage, which could be critical to enabling the use of MHCI tetramers to monitor neoantigen-specific T-cells in the clinic.

Protein Expression and Purification.

HLA and B2M sequences were obtained from Uniprot. org. DNA encoding the signal sequence of HLA and B2M and the extracellular domain of HLA and full length of B2M was synthesized and subcloned into a pET expression vector under the control of the T7 lac promoter. Recombinant HLA and B2M were overexpressed in Escherichia coli, purified from inclusion bodies, and stored in denaturing buffer (6M Guanidine HCl, 25 mM Tris pH 8) at −80° C. After induction of expression, B2M and HLA biomass pellets were resuspended in lysis buffer (PBS+1% Triton X-114) at 5 mL/g and homogenized twice in a microfluidizer at 1000 bar. The homogenized suspension was then spun at 30,000 g for 20 min in an ultracentrifuge. The pellets were collected, washed with 500 mL of 0.5% Triton X-114 in PBS, and centrifuged at 30,000 g for 20 min. The pellet was collected again and washed a second time as described above. The purified inclusion bodies were dissolved in a denaturing buffer (20 mM MES, pH 6.0, 6M Guanidine) at a concentration of 10 mL/g and stirred at 4° C. overnight. The dissolved pellet was centrifuged at 40,000 g for 60 min and the supernatant was collected and filtered through a 0.22 pin filter. The concentration was determined by UV-Vis at 280 nm using the protein's extinction coefficient. Samples were then snap-frozen and stored at −80° C. prior to generation of MHCI complexes.

Peptide Selection for Screening.

Peptides for the initial binding screens were selected from the Immune Epitope Database and Analysis Resource (www.iedb.org). The peptide binders identified in the database were sorted based on affinity, and five peptides with the highest measured affinity were selected. In cases where the peptide sequences were similar for the top 5 (differing by less than four amino acids), the next highest affinity peptides with unique sequences were selected to ensure maximal peptide diversity in the screen (Table 3).

MHCI Refold (Small Scale).

Recombinant HLA alleles and B2M were over-expressed in E. coli, purified from inclusion bodies, and stored under denaturing conditions (6M Guanidine HCl, 25 mM Tris pH 8) in −80° C. as described above. In a 200 μL reaction, the peptide (0.01 mM, per well), oxidized and reduced glutathione (0.5 mM and 4.0 mM, respectively), recombinant HLA alleles (0.03 mg/mL) and B2M (0.01 mg/mL) were all combined in a 96-well plate. Refolds were performed with five different peptides for each HLA of interest as described above (Table 3), and the MHCI complex was incubated at 4° C. for 3-5 days to allow refolding. MHCI complex refolds were also performed in the absence of a peptide and used as the negative control to calculate the signal to noise (S/N) for the experimental peptides. Given that there should have been minimal properly refolded complex in the absence of a peptide, these samples provided the overall background of the assay to calculate the S/N value. HLA-A*02:01 refolded with the CMV pp65 viral epitope was used as a positive control. Peptides yielding the highest signal to noise ratio (S/N) were selected for further analysis.

After identifying the most stable peptide binder based on the ELISA analysis for each HLA allele, peptides were redesigned with a UV-cleavable amino acid (denoted “J”) at different positions along the peptide sequence (Table 4). In brief, variants of the most stable peptide binders identified in the initial screen were redesigned in which the J amino acid was substituted at positions 2, 4, 6, and 8, relative to the N-terminus. Formation of stable conditional MHCI complexes upon refolding with the redesigned peptides were identified by ELISA as described above. The conditional MHCI ligands resulting in the most stable complex based on the ELISA assay readout were used for scaled-up MHCI production. The original peptide (containing no UV amino acid substitution) was used as a positive control. All peptides used here were purchased from JPT (www.jpt.com) or ELIM Biopharm (www.elimbio.com).

ELISA Assay.

Two different ELISA assays were evaluated to optimize the sensitivity of the assay. In the first assay format, the refolded MHCI was captured with anti-B2M antibody and detected with pan ABC anti-HLA antibody (clone W6/32). In the second assay format, MHCI was captured with the pan ABC anti-HLA antibody (clone W6/32) and detected with the anti-B2M antibody. In both assays, a 384-well Nunc Maxisorp plate (Thermo Fisher Scientific, Waltham, MA) was coated with 25 μL/well of capture antibody, mouse IgG1 anti-human B2M (BioLegend, San Diego, CA) or mouse IgG2a anti-HLA ABC clone W6/32 (Novus Biological, Littleton, Co.), at 8 μg/mL in coating buffer (0.05 sodium carbonate pH 9.6). After overnight incubation at 4° C., the plate was washed 3 times with wash buffer (PBS, 0.5% Tween 20). The plate was then blocked with 50 μL/well of Block buffer (PBS, 0.5% BASE, 10 ppm Proclin) and incubated at room temperature (RT) with agitation for 1 h. After washing the plate 3 times with wash buffer, 25 μL/well of the unpurified refolded MHC complex at 40 μg/mL with and without peptides in Assay diluent (PBS, 0.5% BSA+0.05% Tween 20+10 ppm Proclin) was added to the plate and incubated for 1 hour at RT. The plate was washed 6 times and 25 μL of biotinylated mouse IgG2a anti-HLA ABC clone W6/32 (Novus Biological, Littleton, Co.) (assay format 1) or biotinylated mouse IgG1 anti-human B2M (BioLegend, San Diego, CA) (assay format 2) at 100 ng/mL in assay diluent was added to each well. After a 1 h incubation at RT and six washes, 25 μL/well of Streptavidin-Horseradish Peroxidase (GE, Marlborough, MA) was added to the plate and incubated for 30 min at RT. The color reaction was developed with TMB peroxidase substrate (Moss, Pasadena, MD) at RT for 15 min, and the reaction was stopped with 1 M phosphoric acid. The OD absorbance values were measured at a wavelength of 405 nm with a reference at 620 nm. Refolded MHC monomers without peptide were included in each assay to measure the background of the assay and calculate the signal to noise (S/N) of the experimental peptides.

MHCI Peptide Refold, Biotinylation, and Purification (Large Scale).

In a 1, 5, or 15 L reaction, the selected peptide (0.01 mM), oxidized and reduced glutathione (0.5 mM and 4.0 mM, respectively), recombinant HLA (0.03 mg/mL) and B2M (0.01 mg/mL) were combined in refold buffer (100 mM Tris, pH 8.0, 400 mM L-Arginine, 2 mM EDTA). The refold mixture was then stirred for 3-5 days at 4° C., filtered through a 0.22 μm filter, and concentrated and buffer exchanged by tangential flow filtration (TFF) (Millipore P2C010001) into 25 mM Tris pH 7.5. The protein components were analyzed by LC/MS to ensure that the HLA was in the appropriate reduced state. The concentrated and refolded MHCI complex was then biotinylated through the addition of BirA (1:50 [wt:wt] enzyme:MHCI), 100 mM ATP and 10× reaction buffer (100 mM MgOAc, 0.5 mM biotin). The biotinylation reaction was mixed for 2 hat room temperature. The sample was dialyzed and analyzed by LC/MS to quantify biotinylation. The biotinylated MHCI complex was purified by anion exchange chromatography on an AKTA Avant FPLC using a 1 or 5 mL HiTrap Q HP column, depending on the reaction size. The column was equilibrated with 10 column volumes (CV) of 25 mM Tris HCl, pH 7.5 at a flow rate of 5 mL/min. The MHCI complex was loaded on the column at a 5 mL/min flow rate and eluted using 0-60% gradient of buffer B (2.5 mM Tris HCl, pH 7.5, 1 M NaCl) over 30 CV. Fractions across the eluted peak were run on SDS-PAGE, and fractions containing both B2M and HLA bands were pooled. Pooled fractions were buffer-exchanged into storage buffer (25 mM Tris HCl, pH 8.0, 150 mM NaCl). Protein concentration was determined by UV absorbance at 280 nm, and samples were snap-frozen and stored at −80° C.

Liquid Chromatography/Mass Spectrometry (LC/MS) Analysis.

Between 2 and 5 μg of MHCI complex was injected on an AdvanceBio RP-mAb diphenyl column, 2.1×75 mm, 3.5 μm (Agilent). The column was heated to 80° C. and exposed to a gradient of 25-40% mobile phase B in 2.0 min at 0.8 mL/min. Mobile phase A was 0.05% TFA in water. Mobile phase B was 0.05% TFA in acetonitrile. The column eluent was sent to an Agilent 6230 ESI-TOF LC/MS for mass spectrometry data acquisition. In order to quantitate MHCI concentration and molar ratios of B2M to HLA, standard curves of B2M and HLA alleles were generated by injecting known amounts of each protein using the method described above. Peak areas at A280 were used to generate standard curves that allowed for the quantitation of the individual protein subunits in MHCI complexes. HLA and B2M masses were deconvoluted using the MassHunter Qualitative Analysis software (Agilent).

Size Exclusion Chromatography-Multi Angle Light Scattering (SEC VIALS) Analysis.

The MW of the MHCI complex was determined as described previously. Briefly, samples at mg/mL were injected (10 μl for A*02:01 MHC; 25 μl for other MHCI alleles) onto a TSKgel SW3000 Analytical SEC column (Tosoh Bioscience), with isocratic gradient of phosphate buffered saline (PBS pH 7.2 with an additional 150 mM NaCl) at ambient temperature, coupled to a multi-angle light scattering system (MALS) (Wyatt Instruments) to measure molar mass.

2D LC/MS analysis. A two-dimensional liquid chromatography mass spectrometry (2D LC/MS) method was used to characterize peptide binding to MHCI complexes. Between 2 and 3 μg of MHCI complexes were injected on the instrument and sent to the first dimension column. The first dimension LC method employed an analytical size exclusion column (SEC) (Agilent AdvanceBio SEC 300 Å, 2.7 μm, 4.6×15 mm) to separate intact complex from excess peptide run at an isocratic flow of 0.7 mL/min in 25 mM TRIS pH 8.0, 150 mM NaCl for 10 min with signal acquisition at 280 nm. A sampling valve collected the entirety of the complex peak that eluted between 1.90 and 2.13 min in a volume of 160 μL and injected it onto the second dimension reversed phase column (Agilent PLRP-S 1000 Å, 8 μm, 50×2.1 mm). The second dimension column was exposed to a gradient of 5-50% mobile phase B in 4.7 min at 0.55 mL/min with the column heated to 80° C. Mobile phase A was 0.05% TFA. Mobile phase B was 0.05% TFA in acetonitrile. The column eluent was sent to an Agilent 6224 ESI-TOF LC/MS for mass spectrometry data acquisition (Agilent Mass Hunter).

ELISA-Based Analysis of MHCI Refold.

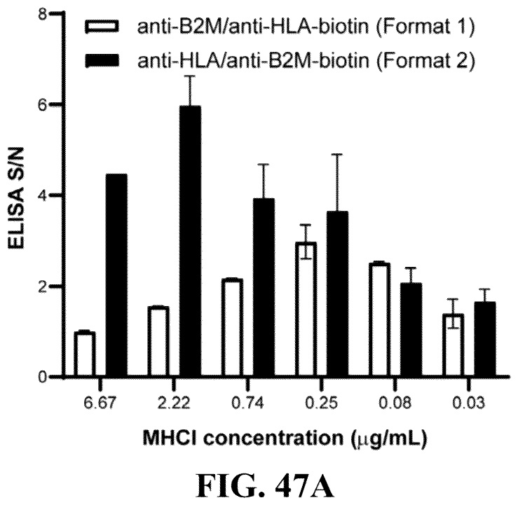

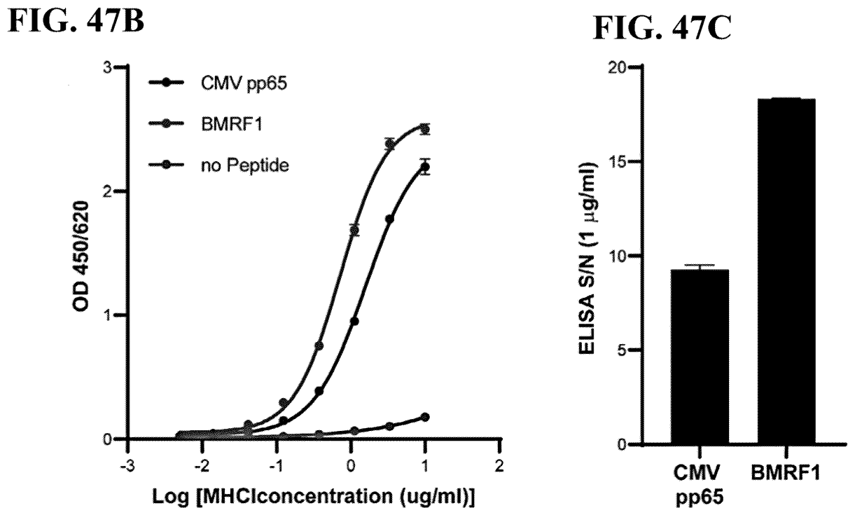

One of the main objectives of this manuscript was to develop a robust HTP workflow for the identification of peptides containing a non-natural UV cleavable amino acid (conditional MHCI ligand) that could form a stable conditional MHCI complex. The first step in this process was to develop an ELISA assay that could measure the formation of a stable MHCI complex after a refold screen. The HTP refold protocol involved mixing denatured recombinant HLA (0.03 mg/mL), B2M (0.01 mg/mL), peptide (0.01 mM), oxidized and reduced glutathione (0.5 and 4.0 mM, respectively) in a 200 μL reaction within a 96-well plate and allowing the refold reaction to proceed for 3-5 days at 4° C. in a deli fridge before ELISA analysis. Since the HLA components are not biotinylated at this stage, we could not use the widely published streptavidin-based ELISA. We instead evaluated two formats 1) capture with anti-B2M and detection with anti-HLA and 2) capture with anti-HLA (clone W6/32) and detection with anti-B2M. In both cases, the detection anti-body was labeled with biotin, and signal generation was induced after the addition of streptavidin-HRP and substrate. CMV pp65 peptide and HLA-A*02:01 were used for these initial screens. S/N values were calculated using MHCI complexes refolded in the absence of a peptide (no peptide control) as a measure of the background of the assay. Although the signal and detection range were comparable for both for mats, the S/N values were much higher for format 2 (FIG. 47A, black bars) than format 1 (FIG. 47A, white bars) at concentrations above 0.25 μg/mL. We believe the higher specificity of format 2 is because the capture step uses an anti-HLA antibody that recognizes a conformational epitope on MHCI and should be selective for only properly folded MHCI. In contrast, the anti-B2M capture is not dependent on properly folded MHCI and will capture complexes with properly folded HLA as well as partially denatured HLA, which we believe accounts for the higher detection signal for no peptide control of format 1. Based on these findings, we selected format 2 for the remaining screens. FIG. 47B shows the ELISA results as a function of MHCI or MHCI-peptide concentration with ELISA. Format two as described in the materials and methods using MHCI molecules refolded with CMV pp65 peptide, BMRF1 peptide and no peptide (background control). As the MHCI concentration was increased, we observed an increase in the OD450/620 ELISA signal for both the CMV pp65 and BMRF1 peptides but little to no increase in signal for the no peptide control, which is consistent with the detection of properly refolded MHCI complex. FIG. 47C shows the S/N of the ELISA analysis using the no peptide control as background at a MHCI concentration of 1 μg/mL for both CMV pp65 and BMRF1. Both antigens had a signal that was ten-fold greater than background, demonstrating that this assay format produces highly sensitive S/N and can be readily used to identify antigens that can form a stable MHCI complex during the refold step. In addition, the standard deviations were very small (FIGS. 47B, 47C) indicating that the assay is highly reproducible for a given experiment and can be used to reliably select optimal peptides without the need to perform the assay in duplicate. The goal of assay optimization was to develop an assay that was highly reproducible and did not require running in duplicate or triplicate to ease screening of 100s of alleles and based on these results we believe this has been achieved.

Identification of Peptide Binders and Conditional MHCI Ligands Across HLA Alleles.

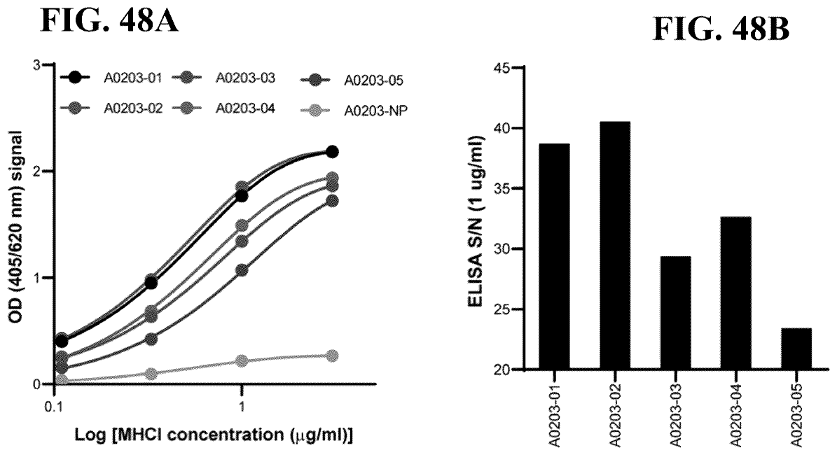

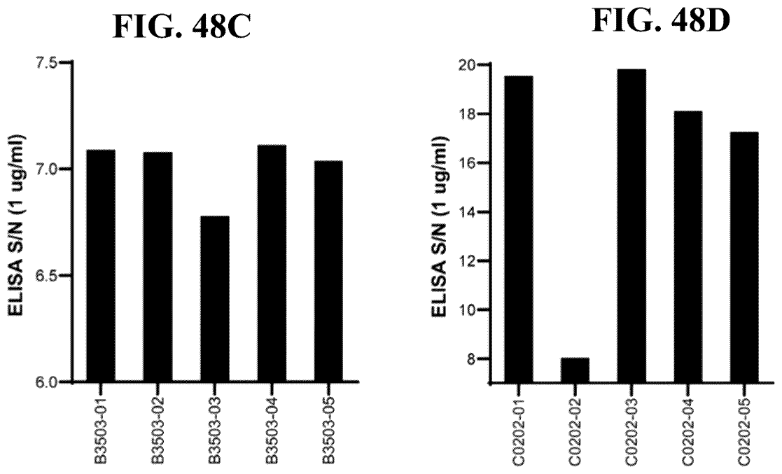

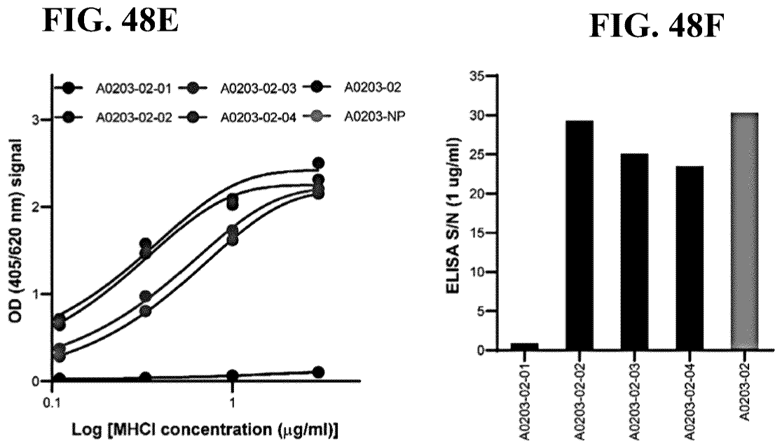

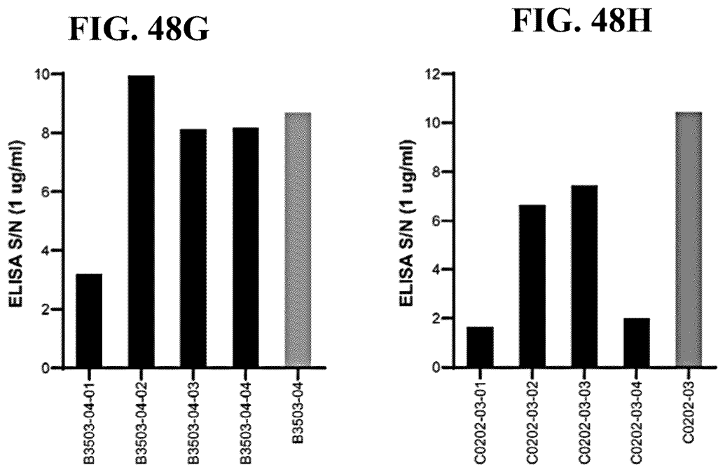

Peptides were selected from IEDB (Table 3). The ability of these peptides to form a stable MHCI complex with the corresponding HLA allele was analyzed using the ELISA assay described above. Titration curves for the refolded MHCI complexes generated with the five peptides and HLA-A*02:03 are shown in FIG. 48A. The assay was performed at MHCI concentrations ranging from 0.1 to 3.0 μg/mL and, as observed for the positive controls in FIG. 47B, there was an increase in the ELISA OD at increasing MHCI concentrations and the signal began to saturate above 1 μg/mL. In addition, we observed only a minimal increase in signal for the negative control across the titration range. The 1.0 μg/mL concentration was selected to compare the S/N ELISA values for the A*02:03, B*35:01, and C*02:02 alleles because it was slightly below saturation (EC60-EC85 depending on peptide-HLA combination) (FIGS. 48B-48D). The S/N background for the five peptides screened against A*02:03 was relatively high, with values ranging from 20 to 40. This suggests that all peptides selected from IEDB were not only binders, but could also form stable MHCI complexes upon refolding. The A*02:03-02 peptide yielded the highest S/N value and was selected for the design of UV peptides. For B*35:03, the S/N value from the selected peptides also had a relatively high S/N background, ranging from 6.75 to 7.25 (FIG. 48C). B*35:03-04 yielded the highest OD value and was subsequently selected to design candidate conditional MHCI ligands. The S/N values for the C*02:02 peptides were all between 18 and 20 except C*02:02-02, which was much lower (˜8) (FIG. 48D). C*02:02-03 yielded the highest S/N value and was selected to design candidate conditional MHCI ligands. A*26:01, B*18:01, and C*14:02 were also tested and similar results were observed (FIGS. 54A-54F). Although the S/N for all alleles tested was relatively high and provided confidence that the assay was identifying peptides that formed a stable complex, there were significant differences in the magnitude of the S/N signal across the alleles. We believe these results are likely due to the variability in the affinity of the pan-HLA antibody for the different HLA alleles. In contrast, the variability within a given allele (e.g., A*02:03 and C*02:02) was likely due to difference in peptide affinity across the different peptides screened.

Variants of the top-performing peptides in the initial screen (i.e., A0203-02, B3503-04, and C0202-03) (FIGS. 48B-48D) were designed with a UV-cleavable amino acid (denoted J) substituted at positions 2, 4, 6, and 8 from the N-terminus to identify UV-peptides that form stable complexes across the different HLA alleles. A titration curve of MHCI complexes assembled with the four conditional MHCI ligands screened derived from the A0203-02 peptide are shown in FIG. 48E. As was observed for the non-UV cleavable peptides, an increase in the ELISA OD was observed with increasing MHCI concentration and the values started to saturate at 1 μg/mL (FIG. 48E). The ELISA S/N for all conditional MHCI ligands across the different alleles is shown in FIGS. 48F-48H. All conditional MHCI ligand variants with J amino acid substitution at position 2 showed very low S/N ELISA values (FIGS. 48F-H), indicating minimal or no MHC complex formation (A0203-02-01, B3503-05-01, C0202-03-01, and C0202-03-04) relative to the parent peptide (gray bar FIGS. 48E-48F). This finding was expected since this position is known to be a MHCI-peptide anchor position. All other conditional MHCI ligands screened for A0203-02 and B3503-04 yielded S/N values similar to the parent peptide (FIGS. 48F-48G). In contrast, all conditional MHCI ligands for C0202-02 had lower OD values when compared to the parent; however, the S/N for C0202-02-02 and C0202-02-03 were still relatively high at 6 and 8, respectively (FIG. 48H), indicating that the UV cleavable amino acid had a slightly negative impact on MHCI stability. The conditional MHCI ligands resulting in the highest S/N value (A0302-02-02, B3503-04-02 and C0202-03-03) were then selected for scaled-up production. A similar analysis was performed to identify optimal conditional MHCI ligands for A*26:01, B*18:01, and C*14:02 alleles (FIGS. 54A-54F).

Scaled-Up Refold and Purification of MHCI Monomers.

Generation of recombinant MHCI complexes through refolding of denatured B2M and HLA purified from E. coli inclusion bodies in the presence of the peptide of interest was initially developed over 20 years ago. Since this initial report, there have been many studies and methods developed for generation and purification of MHCI complexes using a refold protocol. The vast majority of these methods include some variation of the following process: (1) mix HLA and B2M with peptide in generic refold buffer with redox/oxidation agents (incubation time may vary from 1 to 5 days), (2) filter refolded material to remove aggregates, (3) concentrate down to volume compatible with size exclusion chromatography (SEC) (1-2 mL depending on the column), (4) purify using SEC, (5) biotinylate purified refolded MHCI complex, and (6) purify of MHCI complex after the biotinylation reaction (SEC, spin column filters, etc.). The majority of these production methods are only practical at a 1 L scale or smaller and yield only a few milligrams of refolded material; therefore, numerous production runs would be required to produce sufficient material for use in supporting a clinical program. Multiple runs could potentially result in lot-to-lot variability, which may confound interpretation of downstream tetramer staining data. To address these limitations, we developed a novel workflow to enable MHCI refold and purification to be scaled up to 15 L.

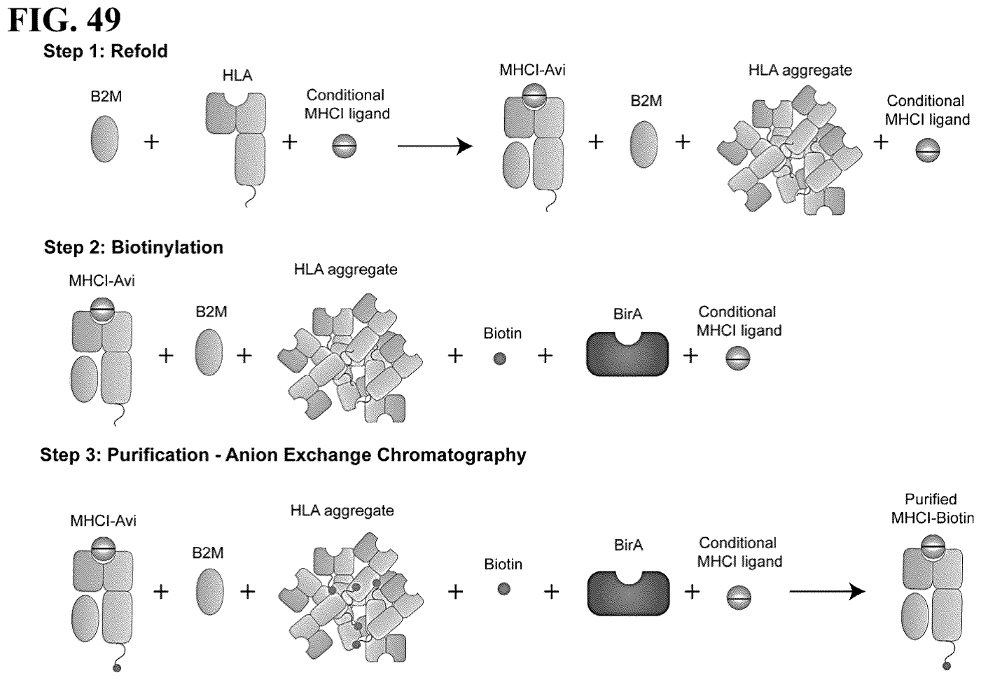

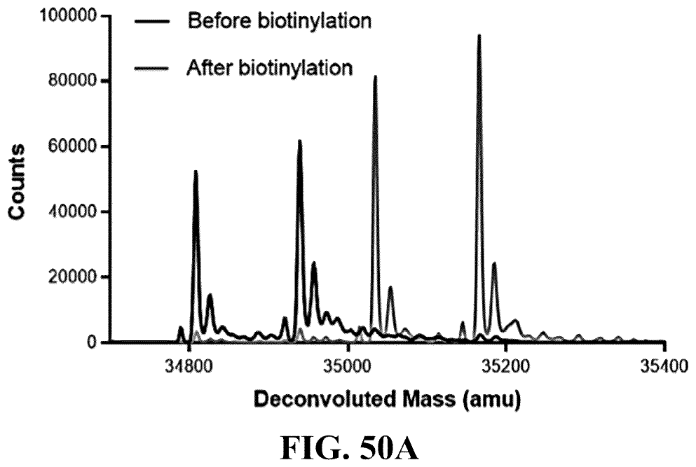

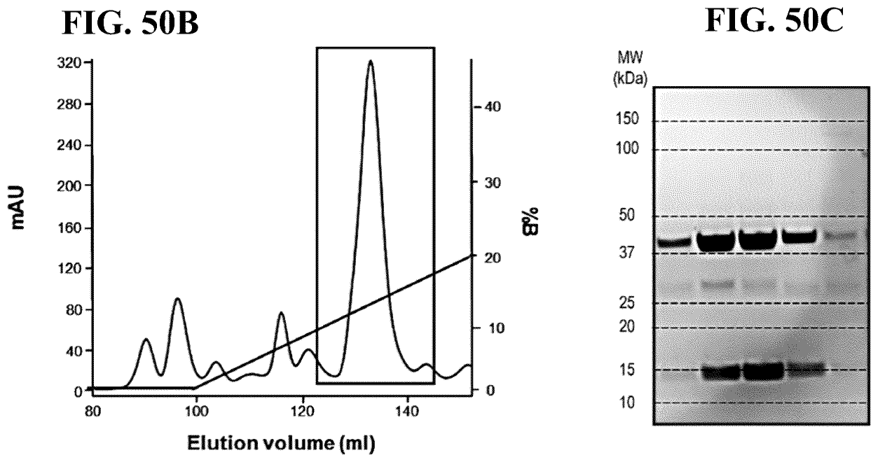

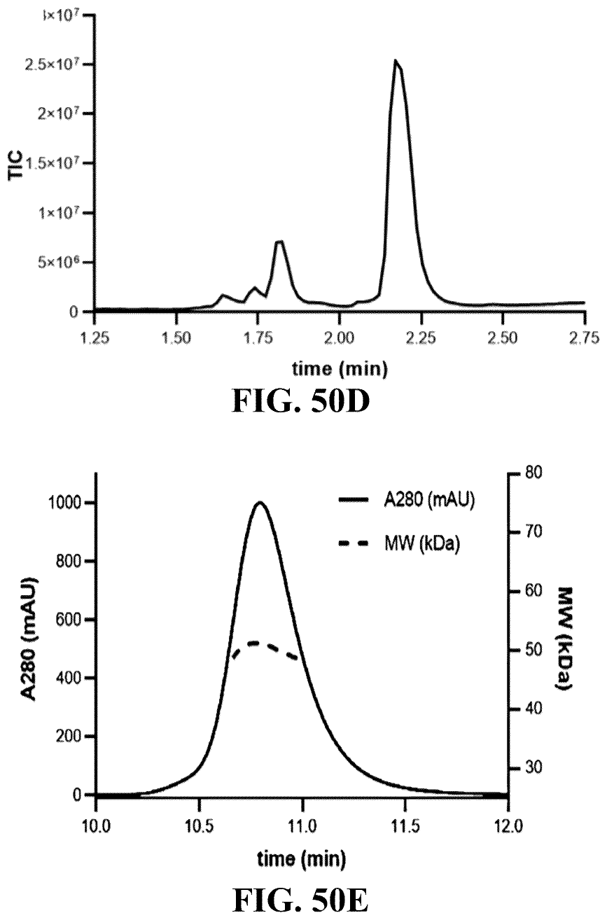

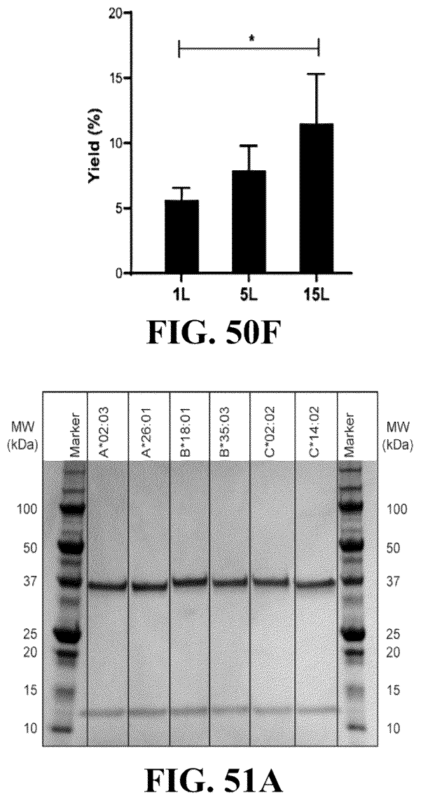

A schematic of the refold and purification protocol developed in this study is shown in FIG. 49. For method optimization, the published HLA-A*02:01 specific conditional MHCI ligand were used. The primary limitation to scaled-up production is the need for an SEC purification step, which requires highly concentrated samples. In addition, the biotinylation step is typically performed on purified refolded MHCI complexes and requires a secondary purification step, which further limits scaled-up production. To address these limitations, we developed a three-step production process that includes the refold reaction, in-process biotinylation, and anion exchange chromatography purification (FIG. 49). LC/MS analysis of the HLA component of the MHCI complex before and after the biotinylation step is shown in FIG. 50A. The black line shows the HLA protein before biotinylation. Two peaks were observed at 34,812 and 34,943 Da, which correspond to the HLA protein with and without the N-terminal methionine group. These two populations are likely caused by incomplete modification and subsequent removal of formylmethionine and the N-terminal methionine by formylmethionine deformylase and methionine aminopeptidase (MAP), respectively, which can vary depending on the adjacent amino acids, and in some cases the N-terminal methionine is not removed. Therefore, it is likely that the N-terminal HLA sequence was not ideal for total MAP activity, so only partial N-terminus removal was observed. After biotinylation, we observed an increase in the mass for both peaks of −244 Da, which corresponds to the mass of biotin (FIG. 50A). No residual peaks were observed at the nonbiotinylated masses, indicating 100% biotinylation. These combined results demonstrate the feasibility of adopting an in-process biotinylation step to eliminate the need for two purification steps.

After completion of the biotinylation step, the resulting biotinylation reaction was buffer exchanged into 25 mM Tris (pH 8.0) and prepared for purification via anion exchange chromatography. Anion exchange was selected for purification over SEC because it is amenable to direct loading of the large volumes used during the biotinylation step (10-100 mL). A representative Q-HP anion chromatogram and gradient for a 1 L refold is shown in FIG. 50B. A large peak was observed at an elution volume of ˜130 mL with several smaller peaks, likely representing minor contaminants from the inclusion body purification. Fractions from the major peak were run on an SDS-PAGE gel, and bands corresponding to the expected MW for HLA-A*02:01 and B2M were observed across the peak (FIG. 50C). Fractions were pooled based on the presence of bands at the expected molecular weights for HLA and B2M in the SDS-PAGE analysis and run on LC/MS and SEC-MALS. The TIC chromatogram of the purified biotinylated MHCI complex is shown in FIG. 50D. The two adjacent peaks at a retention times of 1.7 and 1.75 min corresponded to the R and S diastereomers of the conditional MHCI ligand resulting from the use of a racemic UV amino acid. The peaks at retention times of 1.8 min and 2.2 min correspond to B2M and HLA-A*02:01, respectively. Standard curves of both B2M and HLA-A*02:01 were generated, and the area under the curve was used to quantify the molar concentration and molar ratios of B2M to HLA. If the refold process resulted in the proper pairing of B2M to HLA, the molar ratios of the two components should be close to 1. For this preparation, the ratio was calculated to be 0.95, suggesting proper pairing. The MHCI complex was further analyzed by SEC-MALS for native mass analysis to further confirm proper 1:1 HLA:B2M pairing and monodispersity of the sample. The A280 SEC chromatogram peak was highly symmetric, indicative of a homogeneous and monodisperse protein sample, and no aggregate peak was observed (FIG. 50E). The MW across the MHCI peak ranged from 48.8 to 51.3 kDa (FIG. 50E, red dashed line) and the average value was 49.1 kDa, close to the expected MW of the MHCI complex (48.1 kDa). The collective LC/MS and SEC-MALS analyses suggest that the refold and purification protocol yielded a highly pure and properly folded MHCI complex (FIG. 50F).

One of the primary goals of developing a novel purification workflow was to enable scaled-up production. To test scalability, the optimized 1 L (40 mg of HLA and B2M starting material) protocol was performed at the 5 L (200 mg) and 15 L (600 mg) scales. The refold was quantified at these production scales by calculating the mass of final purified refolded material relative to the mass of material added to the refold reaction (% yields±standard deviations) (FIG. 50F). Interestingly, we observed a gradual increase in average % yield as the process was scaled up from 1 L (˜6%) to 5 L (˜8%) to 15 L (˜11%) and the difference in yield between the 1 L and 15 L scale was found to be statistically significant (p-value<0.05). The amount of MHCI complex generated from the 1 L scale and 15 L scale was −2.4 mg and ˜60 mg, respectively, which corresponds to a 25-fold increase in material generation per refold at the 15 L scale. These combined findings demonstrate the feasibility of scaling up the production and purification of MHCI complexes using the workflow described in this study.

Scaled-Up Production and Peptide Exchange Analysis of Six MHCI Monomers with UV Peptides Identified from the HTP Screen.

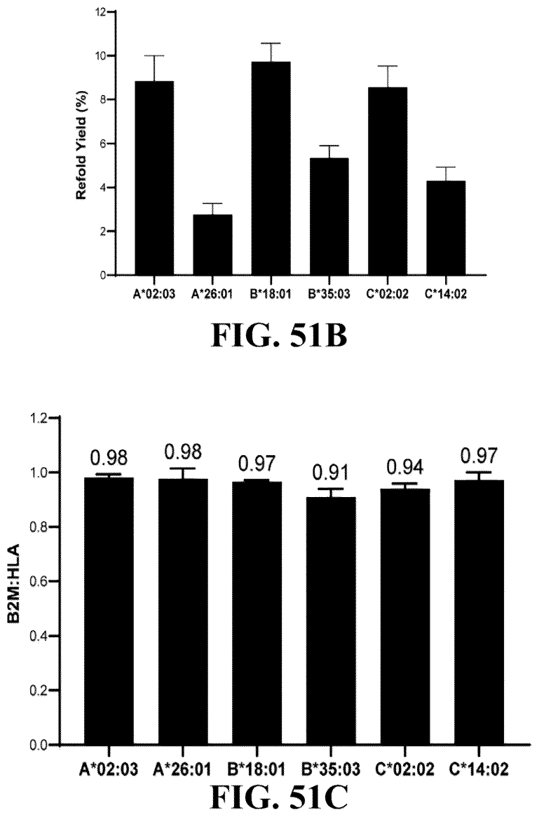

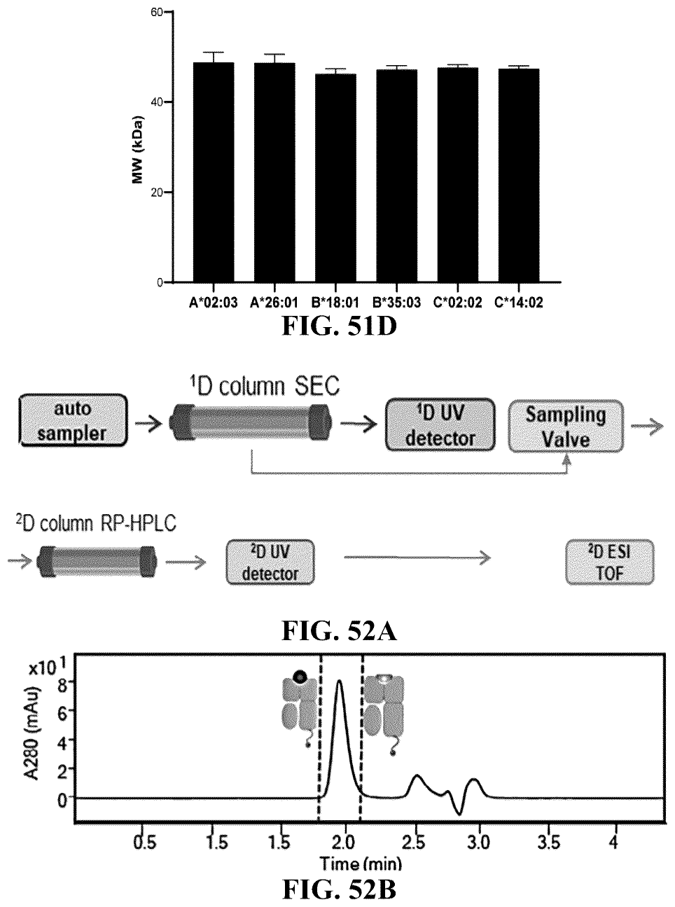

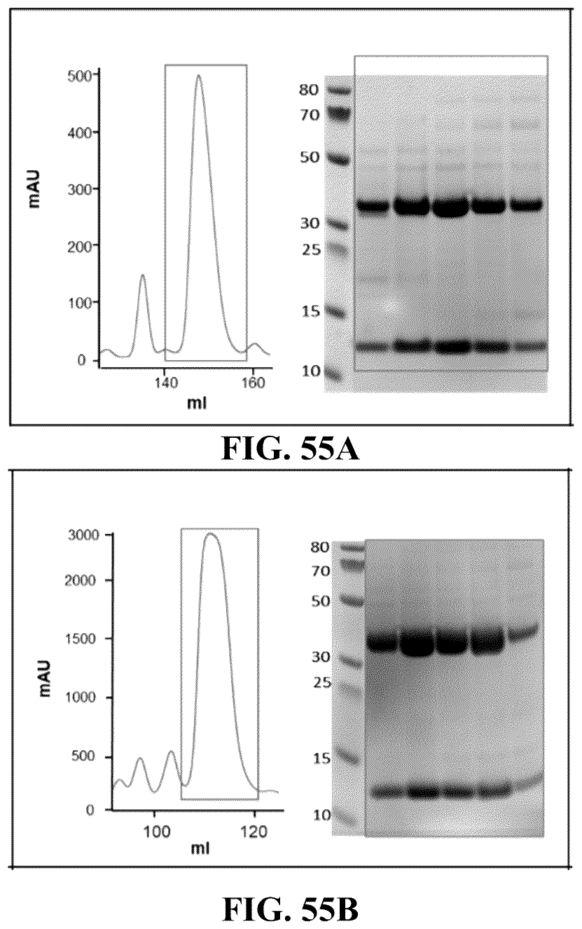

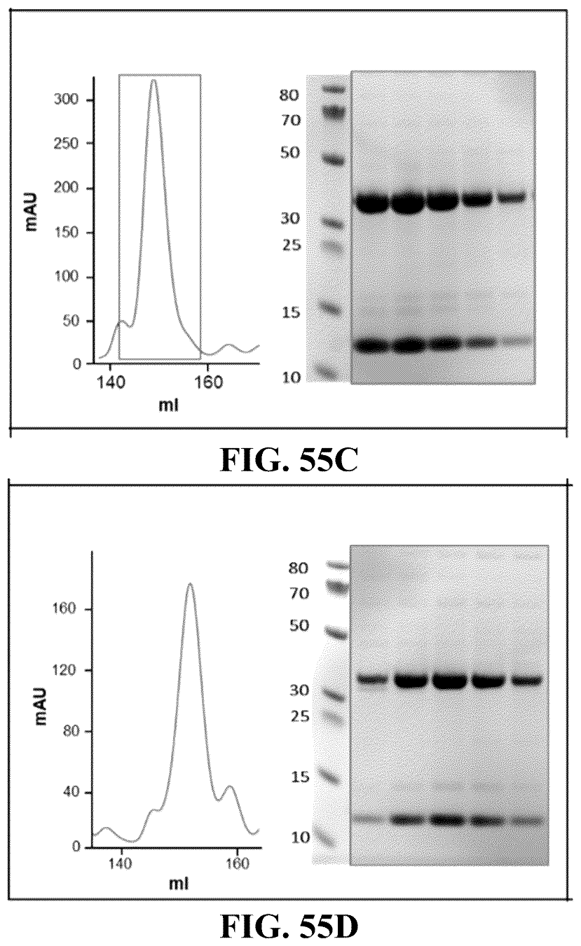

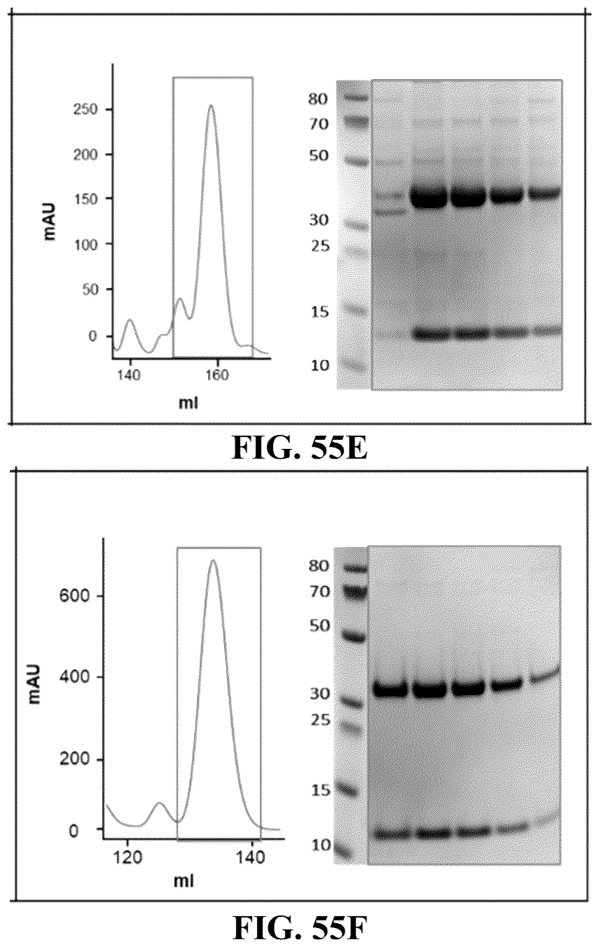

The refold and purification protocol described above was applied to the large-scale production of MHCI complexes with the conditional MHCI ligands identified in the HTP screen. The Q-HP anion chromatograms of all six constructs and corresponding SDS-PAGE of pooled fractions are shown in FIGS. 55A-F. The chromatograms for these refolds are very similar to HLA-A*02:01 (FIG. 50B), with clear HLA and B2M bands in the SDS-PAGE. The pooled fractions for each HLA allele were run on SDS-PAGE, and bands corresponding to HLA and B2M were observed with high purity (FIG. 51A). The % yields for the 1 L refold varied from sample to sample, where A*02:03, B*18:01, and C*02:02 had the highest yields, ranging from 8% to 11%, followed by B*35:03 (˜5%), C*14:02 (˜4%) and A*26:01 (˜2.5%) (FIG. 51B). This variability is likely due to differences in amino acid sequence content and susceptibility to aggregation during refold as well as the ability of the peptide to form a stable complex. Although there was variability, the lowest yield of 2.5% still produced 1 mg of material from a 1 L refold and this could scale to >15 mg at the 15 L scale, which is sufficient to cover >30,000 tetramer stains. LC/MS and SEC-MALS analyses were performed to further evaluate the quality of these MHCI monomers and assess whether the B2M and HLA were properly paired. The results of the LC/MS analysis of the B2M: HLA ratio across the six alleles is shown in FIG. 51C. As was observed for the HLA-A*02:01 MHCI complex, the B2M:HLA ratios for all these samples were close to 1. The MHCI complex was further analyzed by SEC-MALS for intact native state mass analysis. The average MW across the MHCI peak for all six MHCI complexes analyzed is shown in FIG. 51D. The MW for all six constructs ranged from 47.4 to 48.8 kDa, which is well within the expected mass range of these complexes (47.5-48 kDa). These combined findings demonstrate that not only was the refold protocol and purification workflow broadly applicable, but also that the conditional MHCI ligands identified in the small-scale HTP assay were able to form stable MHCI complexes upon scale-up.

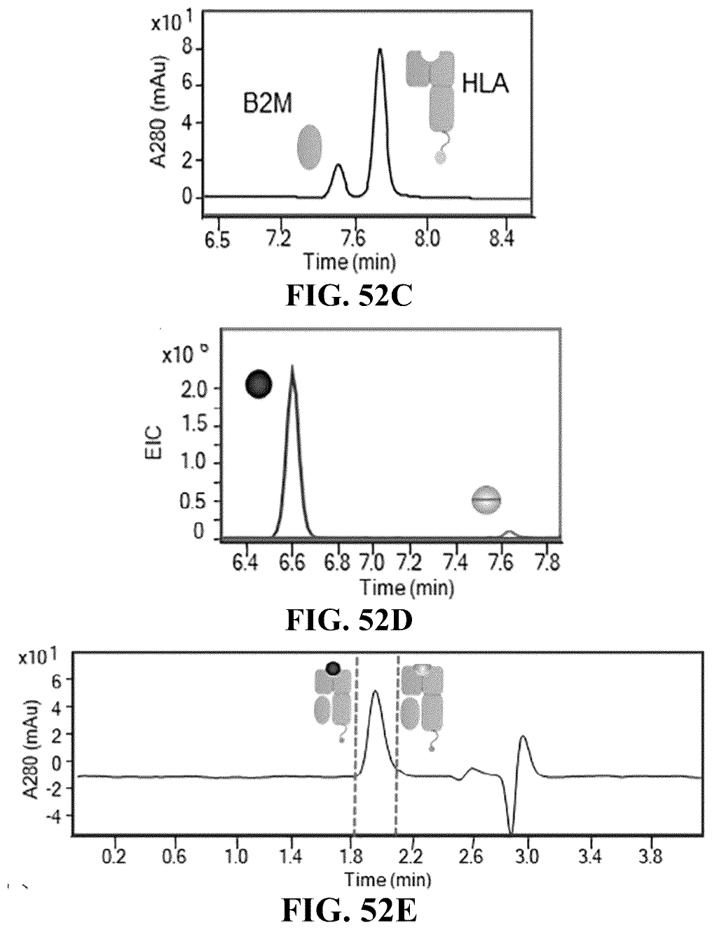

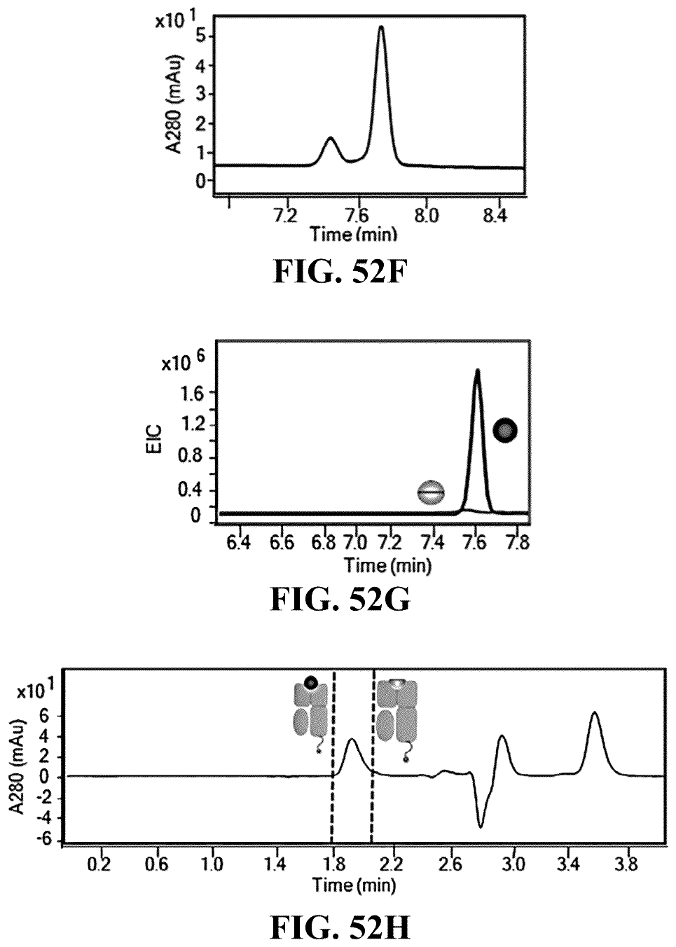

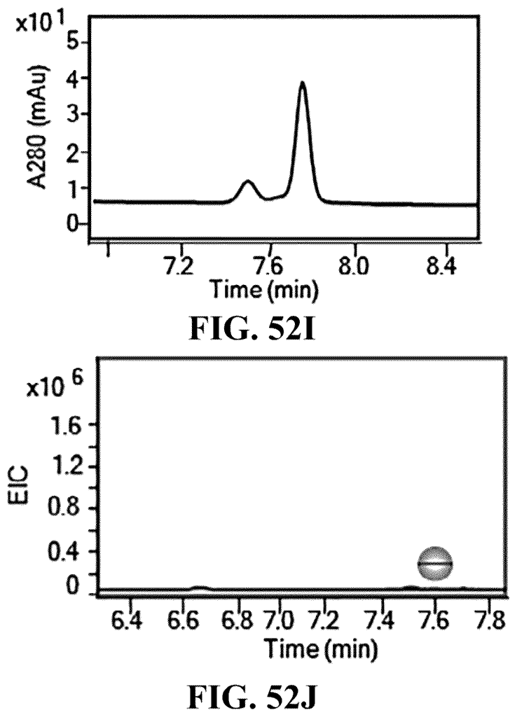

In addition to identifying novel conditional MHCI ligands that could be used for scaled-up production of MHCI monomers, we also wanted to demonstrate that these complexes could undergo peptide exchange upon UV exposure to enable HTP generation of MHCI complexes. One of the most widely used methods to measure peptide exchange of the conditional MHCI ligands after UV exposure is ELISA. Although this assay has proven valuable in demonstrating peptide exchange upon UV exposure, the assay is semi-quantitative and does not provide a direct measurement of the exchanged peptide, nor does it allow for quantification of the cleaved peptide. To address these limitations, we developed a 2D LC-MS analysis method for direct quantification of the peptide loaded into the complex during the exchange process. A schematic of the assay is shown in FIG. 52A. The first step (first dimension) in this method is injection of the peptide exchange reaction mixture (MHCI complex after exposure to UV+100-fold molar excess of exchanged peptide) on an analytical SEC column, which enables col lection of the MHCI complex without excess peptide by a sampling valve. The second step (second dimension) is injection of the material collected from the MHCI peak onto RP-HPLC. The organic phase of the RP-HPLC step results in dissociation and denaturation of the HLA, B2M and peptide contained within the complex, which enables analysis and quantitation of the individual components of the MHCI complex by reading the Absorbance at 280 nm and LC/MS. An example of the first dimension SEC chromatogram of peptide exchange for the control HLA-A*02:01 conditional MHCI ligand with the CMV pp65 epitope is shown in FIG. 52B. The chromatogram shows one dominant peak corresponding to the MHCI complex. A fluctuation in the chromatogram A280 signal was consistently observed between 2.5 and 3 min, which corresponds to the opening and closing of the sampling valve. Since there is a large pressure difference between the first and second dimension, this fluctuation is likely associated with the sudden change in pressure as the valve opens and closes. An example of the A280 chromatogram for the second dimension HPLC step is shown in FIG. 52C. The HLA and B2M peaks are clearly visible, but no A280 peak for the CMV pp65 peptide or conditional MHCI ligand are observed because they do not contain any tryptophan or tyrosine residues and thus no inherent A280 absorbance. To analyze the peptide composition, the extracted ion chromatograms for the exchange peptide and uncleaved conditional MHCI ligand were generated (FIG. 52D). As expected, we observed a large peak corresponding to the CMV pp65 peptide, indicating successful peptide exchange. However, very low levels of the intact conditional MHCI ligand were also observed. This result suggests that either the purified peptide has a synthesis-derived contaminant with a similar mass as the conditional MHCI ligand that complexes to the MHCI molecule and is carried through the 2D LC/MS analysis or a small fraction of the conditional MHCI ligand is protected from UV cleavage when they are in the complex. This peak is also observed when the peptide alone is cleaved in the absence of MHCI, suggesting that this is a contaminant. Regardless, the relative fraction of intact conditional MHCI ligand was very low (˜1%) and should have minimal impact on downstream tetramer staining. The same analysis was performed to verify the presence of cleaved conditional MHCI ligand, and no peaks were detected. By adopting a 2D LC/MS assay to analyze peptide exchange, we were able to measure how the peptide content in the MHCI complex changes during the peptide exchange process. We believe this is a powerful tool that can be used to better understand the parameters relevant to peptide exchange, which cannot be quantified using other traditional methods such as ELISA.

This method was then used to assess peptide exchange with the MHCI complexes generated using the scaled-up production workflow with the novel conditional MHCI ligands identified in the HTP small-scale assay. For each MHCI complex, 4-5 peptides that were found to be binders in the small-scale HTP screen (FIGS. 48B-D, 54A-54C) and 1 known nonbinding peptide were used as positive controls and a negative control, respectively. The first dimension SEC chromatogram for HLA-A*02:03 with peptide A0203-05 showed a clear single peak (FIG. 52E). The second dimension HPLC A280 chromatogram of the peptide exchanged complex also shows the expected B2M and HLA peaks (FIG. 52F). In the EIC analysis of the second dimension, a large peak corresponding to the extracted mass of the A0203-05 peptide was observed (black line FIG. 52G).

However, similar to A*02:01 and CMV pp65 peptide exchange, a small peak corresponding to the intact conditional MHCI ligands was also observed (dashed line FIG. 52G). This phenomenon was seen for all conditional MHCI ligands tested and indicates the presence and carryover of some low level contaminants during the peptide exchange process. It is worth noting that, in all cases, the total fraction of contaminant peptide was less than 1%. The first dimension SEC chromatogram of the HLA-A*02:03 MHCI complex after peptide exchange with a known nonbinder is shown in FIG. 52H. The overall peak area under these conditions was lower than when the peptide exchange process was performed with a peptide binder (FIG. 52E vs. 52H). In addition, the A280 HPLC chromatogram in the second dimension contained two peaks but the overall peak areas were much lower than when a peptide binder was used (FIG. 52F vs. 52I). These combined data suggest that there was significant material loss when the peptide exchange was performed with a nonbinder, likely due to protein aggregation.

Interestingly, a peak at the end of the SEC run (FIG. 52H, retention time −3.6 min) was consistently observed for the negative control exchange samples across all alleles tested, which we believe is due to partially denatured HLA interacting with the column. Finally, in the EIC analysis, no peak corresponding to the A0203 non-binder peptide was observed indicating that peptide exchange did not occur (FIG. 52J). It was surprising that even though no peptide was associated with the complex, a relatively prominent A280 peak was observed in the first dimension, although much smaller than the peak observed with peptide exchange. These results suggest that some of the complex remains associated in the absence of a peptide but it is likely of low quality and not properly folded.

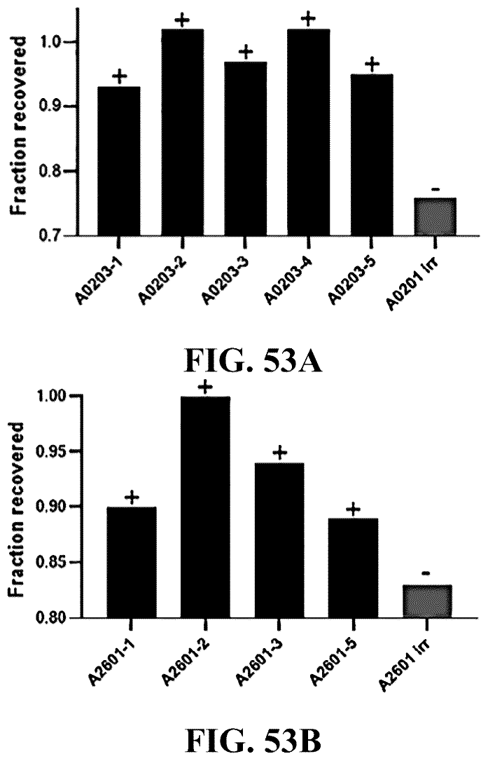

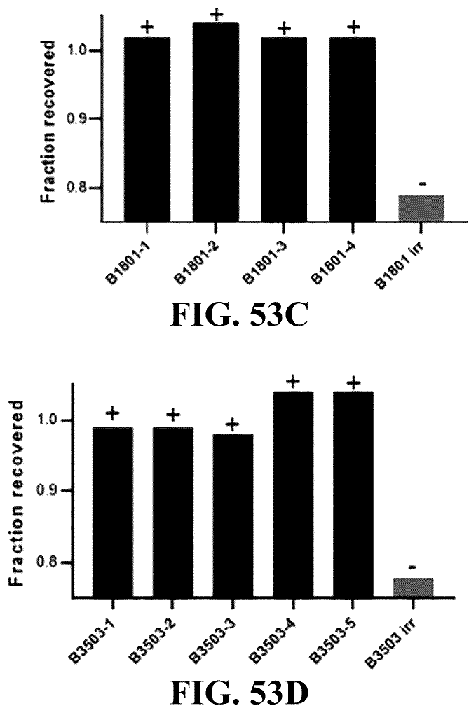

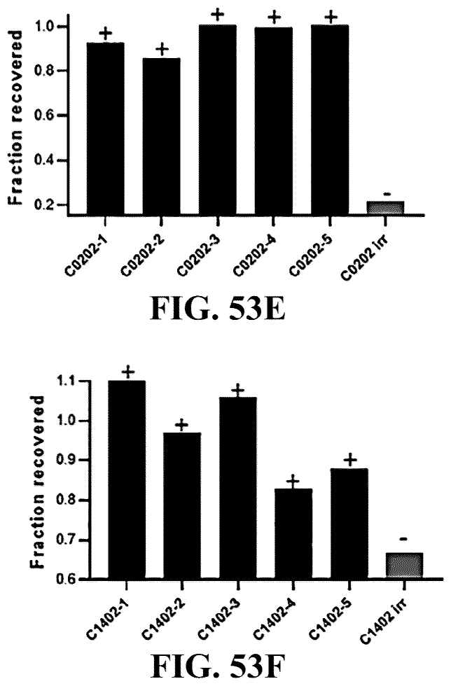

Quantification of the first and second dimension data is shown in FIGS. 53A-53F. For this analysis, we evaluated the fraction of the A280 MHCI peak that was recovered after exchange in the first dimension (SEC) and the presence of the exchanged peptide in the second dimension (EIC). FIGS. 53A-53F shows the fraction of A280 MHCI peak recovered (plotted as a ratio relative to non-peptide exchange control) for the different MHCI complexes across the 4-5 positive controls and 1 negative control. All peptides tested, except the irrelevant peptide, were detected in the second dimension. For HLA-A*0203, the fraction recovered for the positive control binder peptides varied from 0.9 to 1 and the exchange peptide was observed in the second dimension for all peptides. As described above, the fraction recovered for the negative control was still relatively high (˜0.76) even though no exchanged peptide, cleaved conditional MHCI ligand and very low levels of intact conditional MHCI ligands (<1%) were observed. This suggests that the complex remains somewhat intact for a period in the absence of a peptide, although it is likely not properly folded given the instability of the HLA allele in the absence of a peptide.

Similar results were observed for A*26:01 (FIG. 53B), B*18:01 (FIG. 53C), B*35:03 (FIG. 53D), and C*14:02 (FIG. 53F). In all cases, the difference in peak area between the positive and negative control in the first dimension was less than 20-25%. In contrast, there was a drastic difference in the negative and positive controls for C*02:02 (FIG. 52E). For this sample, −80% of the material degraded when a known nonbinder peptide was included in the peptide exchange. Although we do not know the exact reason for this, it is likely due to the ability of the different alleles to remain associated with B2M when a peptide has been removed from the peptide groove on HLA. Regardless, these combined results clearly indicate that we have identified conditional MHCI ligands that allow for scaled-up production of the MHCI peptides and subsequent peptide exchange for HTP generation of MHCI monomers and tetramers.

Conclusion. There is a need to develop workflows to expand the number of HLA alleles for which we have identified conditional MHCI ligands to improve the coverage of MHCI tetramer analysis of patient responses to CI/IO therapies. The methods outlined in this manuscript provide a blueprint for expansion across a broad range of HLA alleles. All conditional MHCI ligands selected using this workflow resulted in relatively high-yield and high-quality conditional MHCI complexes upon scale up. Based on the combined findings of this study, we believe this approach has the potential to be generally applied and enable HTP generation of MHCI monomers and tetramers across a wider range of HLA alleles, which could be critical to enabling the use of MHCI tetramers to monitor neoantigen-specific T-cells in the clinic.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the claims.

TABLE 1
Mass Spec parameters for SEC-native MS and CZE-native MS

| | Protein-Lignand | BsIgG on | MHCI on | MHCI on |
|---|---|---|---|---|
| Mass Spec Instrument Parameters | on SEC-MS** | SEC-MS** | SEC-MS | CZE-MS |
| Sheath gas flow rate | 15 | 40 | 4 | 2 |
| Aux gas flow rate | 4 | 20 | 0 | 0 |
| Spray voltage (kV) | 3.9 | 4 | 4 | 0 |
| Capillary temperature (C) | 325 | 320 | 320 | 250 |
| S-lens RF level | 200 | 200 | 200 | 200 |
| Scan range (m/z) | 1000-10000 | 300-20000 | 350-10000 | 1500-6000 |
| In-source CID (eV) | 0 | 100 | 0 | 75 |
| CE | 0 | 0 | 0 | 0 |
| Resolution (m/z) | 17500 | 17500 | 8750 | 17500 |
| Polarity | Positive | Positive | Positive | Positive |
| Microscanes | 10 | 10 | 10 | 3 |
| AGC target | 3E+06 | 1E+06 | 3E+06 | 3E+06 |
| AGC mode | Fixed | Fixed | Fixed | Fixed |
| Averaging | 0 | 0 | 0 | 0 |
| Maximum injection time (ms) | 50 | 50 | 50 | 20 |
| Source DC offset (V) | 25 | 25 | 25 | 15 |
| Injection flatapole lens (V) | 8 | 8 | 8 | 9 |
| Inter-flatapole lens (V) | 7 | 7 | 7 | 8 |
| Bent flatapole DC (V) | 6 | 6 | 6 | 10 |
| Transfer multiple DC tune offset (V) | 0 | 0 | 0 | 0 |
| C-trap entrance lens tune offset (V) | 0 | 0 | 0.8 | 0 |
| Trapping gas pressure | 2 | 2 | 3 | 2 |

* C. Ren et al., Analytical Chem., 91: 903-911 (2019);
**K.K. Joshi et al., MAbs 11: 1254-65 (2019)

TABLE 2
Parameter descriptions for Table 1

| Mass Spec Instrument Parameters | Parameter description/signifigance |
|---|---|
| Sheath gas flow rate | Entrance to MS (source); aids in desolvation & sensitivity |
| Aux gas flow rate | Source: affects evaporation & cooling, similar fxn to sheath gas |
| Spray voltage (kV) | V applied on outer surface of capillary, ionizes droplets' |
| Capillary temperature (C.) | Temp where ions enter MS; too hot could induce dissociation of complex, too cool effects desolvation |
| S-lens RF level | Focuses ions into a beam |
| Scan range (m/z) | Lowest & highest range of ions surveyed |
| In-source CID (eV) | Results in collusions of analytes in flatapole |
| CE | Energy applied in HCD cell (matters because too much would dissociate complex, too little has poor signal) |
| Resolution (m/z) | We are set at moderate resolution: this parameter shows that diff between SEC and ZipChip is that you can |
| | get higher resolution (ie. distinction between ions) so presumably better data on ZipChip without loss of signal |
| Polarity | All proteins are read in positive mode, no important |
| Microscanes | The number of mini-scans which equal a single ‘scan’; affects time and signal amplitude |
| AGC target | The maximum number of ions allowed into detector at one time (affects sensitivity) |
| AGC mode | AGC target can be a set number (fixed) or variable (ie. by time) |
| Averaging | The sum of many microscans; important with large molecules of proteins & protein complexes |
| Maximum injection time (ms) | How long it takes to fill the ion trap |
| Source DC offset (V) | Combination of 3 voltages |
| Injection flatapole lens (V) | Ion focusing, affects how ions travel through the spectrometer |
| Inter-flatapole lens (V) | Hole where ion beam passes, affects how ions travel through the spectrometer |
| Bent flatapole DC (V) | Affects how ions travel through the spectrometer (where neutral ions are discarded) |
| Transfer multiple DC tune offset (V) | Ion focusing lens with voltage |
| C-trap entrance lens tune offset (V) | Where ions sit before entering orbitrap detector, parameter used for ion heating & cooling |
| Trapping gas pressure | Gas in HCD cell |

TABLE 3
IEDB selected peptides for initial refolding screen.

| Peptide | Peptide | | Organism | Assay | Assay | | | | | |
|---|---|---|---|---|---|---|---|---|---|---|
| ID | seq | Antigen | name | method | Group | Units | Measure | Reference | Date | Title |
| A0203- | FLPSDF | C protein | Hepatitis B | purified | half maximal | nM | 5.9 | J Clin | 1997 | Human histocompatibility |
| 01 | FPSV | | virus | MHC/ | inhibitory | | | Invest | | leukocyte antigen-binding |
| | | | | competitive/ | concentration | | | | | supermotifs predict broadly |
| | | | | radiaoactivity | (IC50) | | | | | cross-reactive cytotoxic T |
| | | | | | | | | | | lymphocyte responses in |
| | | | | | | | | | | pateints with acute hepatitis. |
| A0203- | FMYSD | polymerase | Influenza A | purified | dissociation | nM | 2 | Hum | 2010 | Identification of broad |
| 02 | FHFI | PA | virus | MHC/ | constant KD | | | Immunol | | binding class I HLA |
| | | | | competitive/ | (~IC50) | | | | | supertype epitopes to |
| | | | | radiaoactivity | | | | | | provide universal coverage |
| | | | | | | | | | | of influenza A virus. |
| A0203- | NMLST | RNA-directed | Influenza A | purified | half maximal | nM | 11 | J Virol | 2008 | Immunomic analysis of the |
| 03 | VLGV | RNA | virus | MHC/ | inhibitory | | | | | repertoire of T-cell |
| | | polymerase | | competitive/ | concentration | | | | | specificities for influenza A |
| | | subunit P1 | | radiaoactivity | (IC50) | | | | | virus in humans. |
| | | (PB1) | | | | | | | | |
| A0203- | NLFDIP | protein F12 | Vaccinia virus | purified | half maximal | nM | 0.25 | Proc Natl | 2005 | HLA class I-restricted |
| 04 | LLTV | | WR | MHC/ | inhibitory | | | Acad Sci | | responses to vaccinia |
| | | | | competitive/ | concentration | | | USA | | recognize a broad array of |
| | | | | radiaoactivity | (IC50) | | | | | proteins mainly involved in |
| | | | | | | | | | | virulence and viral gene |
| | | | | | | | | | | regulation. |
| A0203- | GLFGAI | hemagglutinin | Influenza A | purified | dissociation | nM | 1 | Hum | 2010 | Identification of broad |
| 05 | AGFI | precursor | virus | MHC/ | constant KD | | | Immunol | | binding class I HLA |
| | | | | competitive/ | (~IC50) | | | | | supertype epitopes to |
| | | | | radiaoactivity | | | | | | provide universal coverage |
| | | | | | | | | | | of influenza A virus. |
| A2601- | YVIRD | polyprotein | Yellow fever | purified | dissociation | nM | 1 | PLoS | 2011 | Human leukocyte antigen |
| 01 | LAAM | | virus | MHC/direct/ | constant KD | | | One | | (HLA) class I restricted |
| | | | | fluorescence | (~EC50) | | | | | epitope discovery in yellow |
| | | | | | | | | | | fever and dengue viruses: |
| | | | | | | | | | | imprantance of HLA |
| | | | | | | | | | | binding strength. |
| A2601- | ETTNW | polyprotein | West Nile | purified | dissociation | nM | 2 | PLoS | 2010 | Identification of CD8+ T |
| 02 | LWAF | precursor | virus | MHC/direct/ | constant KD | | | One | | cell epitopes in the West |
| | | | | fluorescence | (~EC50) | | | | | Nile virus polyprotein by |
| | | | | | | | | | | reverse-immunology using |
| | | | | | | | | | | NetCTL. |
| A2601- | DVVPM | PPE family | Mycobacterium | purified | dissociation | nM | 3 | Immu- | 2011 | Identification of MHC class |
| 03 | VTQM | protein | tuberculosis | MHC/direct/ | constant KD | | | nology | | II restricted T-cell-mediated |
| | | | | fluorescence | (~EC50) | | | | | reactivity against MHC |
| | | | | | | | | | | class I binding |
| | | | | | | | | | | Mycobacterium tuberculosis |
| | | | | | | | | | | peptides. |
| A2601- | EISGSS | polyprotein | Yellow fever | purified | dissociation | nM | 3 | PLoS | 2011 | Human leukocyte antigen |
| 04 | ARY | precursor | virus | MHC/direct/ | constant KD | | | One | | (HLA) class I restricted |
| | | | | fluorescence | (~EC50) | | | | | epitope discovery in yellow |
| | | | | | | | | | | fever and dengue viruses: |
| | | | | | | | | | | imprantance of HLA |
| | | | | | | | | | | binding strength. |
| A2601- | DTITNV | polyprotein | West Nile | purified | dissociation | nM | 4 | PLoS | 2010 | Identification of CD8+ T |
| 05 | TTM | precursor | virus | MHC/direct/ | constant KD | | | One | | cell epitopes in the West |
| | | | | fluorescence | (~EC50) | | | | | Nile virus polyprotein by |
| | | | | | | | | | | reverse-immunology using |
| | | | | | | | | | | NetCTL. |
| B1801- | FEFTSF | RNA-directed | Influenza A | purified | half maximal | nM | 0.1 | J Virol | 2008 | Immunomic analysis of the |
| 01 | FY | RNA | virus | MHC/ | inhibitory | | | | | repertoire of T-cell |
| | | polymerase | | competitive/ | concentration | | | | | specificities for influenza A |
| | | subunit P1 | | radiaoactivity | (IC50) | | | | | virus in humans. |
| | | (PB1) | | | | | | | | |
| B1801- | DEFKPI | Liver stage | Plasmodium | purified | half maximal | nM | 3.9 | J | 2003 | Simultaneous prediction of |
| 02 | VQY | antigen | falciparum | MHC/ | inhibitory | | | Immunol | | binding capacity for |
| | | | | competitive/ | concentration | | | | | multiple molecules of the |
| | | | | radiaoactivity | (IC50) | | | | | HLA B44 supertype. |
| B1801- | AELLA | polymerase | Hepatitis B | purified | half maximal | nM | 5.8 | J | 2003 | Simultaneous prediction of |
| 03 | ACF | | virus | MHC/ | inhibitory | | | Immunol | | binding capacity for |
| | | | | competitive/ | concentration | | | | | multiple molecules of the |
| | | | | radiaoactivity | (IC50) | | | | | HLA B44 supertype. |
| B1801- | DELVD | Isatin-beta- | Vaccinia | purified | half maximal | nM | 16 | Proc Natl | 2005 | HLA class I-restricted |
| 04 | PINY | thiosemi- | virus WR | MHC/ | inhibitory | | | Acad Sci | | responses to vaccinia |
| | | carbazone- | | competitive/ | concentration | | | USA | | recognize a broad array of |
| | | depdendent | | radiaoactivity | (IC50) | | | | | proteins mainly involved in |
| | | protein | | | | | | | | virulence and viral gene |
| | | | | | | | | | | regulation. |
| B1801- | QEILDL | nef protein | Human | purified | half maximal | nM | 52 | J | 2003 | Simultaneous prediction of |
| 5 | WVY | | immuno- | MHC/ | inhibitory | | | Immunol | | binding capacity for |
| | | | deficiency | competitive/ | concentration | | | | | multiple molecules of the |
| | | | virus 1 | radiaoactivity | (IC50) | | | | | HLA B44 supertype. |
| B3503- | HPNEE | polyprotein | Hepatitis C | purified | half maximal | nM | 6.8 | J | 2012 | Immunogenicity and cross- |
| 01 | VAL | | virus subtype | MHC/ | inhibitory | | | Immunol | | reactivity of a representative |
| | | | 1a | competitive/ | concentration | | | | | ancestral sequence in |
| | | | | radiaoactivity | (IC50) | | | | | hepatitis C virus infection. |
| B3503- | IPAEGR | Glyoxylate | Homo sapiens | cellular | ligand | | Positive | J | 2008 | Large scale mass |
| 02 | VAL | reductase/ | | MHC/mass | presentation | | | Immunol | | spectrometric profiling of |
| | | hydroxypyruvate | | spectrometry | | | | | | peptides eluted from HLA |
| | | reductase | | | | | | | | molecules reveals |
| | | | | | | | | | | N-terminal-extended |
| | | | | | | | | | | peptide motifs. |
| B3503- | APEEH | POTE ankyrin | Homo sapiens | cellular | ligand | | Positive | J | 2016 | Comparative Analysis of the |
| 03 | PVLL | domain family | | MHC/mass | presentation | | | Proteome | | Endogenous Peptidomes |
| | | member F | | spectrometry | | | | Res | | Displayed by HLA-B*27 |
| | | | | | | | | | | and Mamu-B*08: Two |
| | | | | | | | | | | MHC Class I Alleles |
| | | | | | | | | | | Associated with Elite |
| | | | | | | | | | | Control of HIV/SIV |
| | | | | | | | | | | Infection. |
| B3503- | TPANY | 2-oxoglutarate | Homo sapiens | cellular | ligand | | Positive | Mol Cell | 2017 | A Molecular Basis for the |
| 04 | FHVL | dehydrogenase- | | MHC/mass | presentation | | | Proteomics | | Presentation of |
| | | like, | | spectrometry | | | | | | Phosphorylated Peptides by |
| | | mitochondrial | | | | | | | | HLA-B Antgens. |
| | | isoform f | | | | | | | | |
| B3503- | YPYPY | ribosomal | Homo sapiens | cellular | ligand | | Positive | Mol Cell | 2017 | A Molecular Basis for the |
| 05 | PHTAA | protein L2 | | MHC/mass | presentation | | | Proteomics | | Presentation of |
| | FSKLIY | | | spectrometry | | | | | | Phosphorylated Peptides by |
| | L | | | | | | | | | HLA-B Antgens. |
| C0202- | DAVPF | | Homo sapiens | cellular | ligand | | Positive | J | 2017 | Unveiling the Peptide |
| 01 | PISL | | | MHC/mass | presentation | | | Immunol | | Motifs of HLA-C and HLA- |
| | | | | spectrometry | | | | | | G from Naturally Presented |
| | | | | | | | | | | Peptides and Generation of |
| | | | | | | | | | | Binding Prediction |
| | | | | | | | | | | Matrices. |
| C0202- | FAAEA | | Homo sapiens | cellular | ligand | | Positive | J | 2017 | Unveiling the Peptide |
| 02 | QKL | | | MHC/mass | presentation | | | Immunol | | Motifs of HLA-C and HLA- |
| | | | | spectrometry | | | | | | G from Naturally Presented |
| | | | | | | | | | | Peptides and Generation of |
| | | | | | | | | | | Binding Prediction |
| | | | | | | | | | | Matrices. |
| C0202- | IAKSGT | | Homo sapiens | cellular | ligand | | Positive | J | 2017 | Unveiling the Peptide |
| 03 | SEF | | | MHC/mass | presentation | | | Immunol | | Motifs of HLA-C and HLA- |
| | | | | spectrometry | | | | | | G from Naturally Presented |
| | | | | | | | | | | Peptidesand Generation of |
| | | | | | | | | | | Binding Prediction |
| | | | | | | | | | | Matrices. |
| C0202- | KIRDLL | | Homo sapiens | cellular | ligand | | Positive | J | 2017 | Unveiling the Peptide |
| 04 | PVM | | | MHC/mass | presentation | | | Immunol | | Motifs of HLA-C and HLA- |
| | | | | spectrometry | | | | | | G from Naturally Presented |
| | | | | | | | | | | Peptides and Generation of |
| | | | | | | | | | | Binding Prediction |
| | | | | | | | | | | Matrices. |
| C0202- | SYMST | | Homo sapiens | cellular | ligand | | Positive | J | 2017 | Unveiling the Peptide |
| 05 | FPLF | | | MHC/mass | presentation | | | Immunol | | Motifs of HLA-C and HLA- |
| | | | | spectrometry | | | | | | G from Naturally Presented |
| | | | | | | | | | | Peptides and Generation of |
| | | | | | | | | | | Binding Prediction |
| | | | | | | | | | | Matrices. |
| C1402- | SYMST | cytochrome | Entamoeba | purified | dissociation | nM | 0.1 | | 2010 | Large scale analysis of |
| 01 | FPLF | b-like protein | histolytica | MHC/direct/ | constant KD | | | | | peptide-HLA class I |
| | | | | fluorescence | | | | | | interactions |
| C1402- | TYLQS | unnamed | Human | purified | dissociation | nM | 0.1 | | 2010 | Large scale analysis of |
| 02 | LASL | protein product | gamma- | MHC/direct/ | constant KD | | | | | peptide-HLA class I |
| | | | herpesvirus 4 | fluorescence | | | | | | interactions |
| C1402- | YYRYP | 6- | Rattus | purified | dissociation | nM | 0.1 | | 2010 | Large scale analysis of |
| 03 | TGESY | phosphofructo- | norvegicus | MHC/direct/ | constant KD | | | | | peptide-HLA class I |
| | | 2- | | fluorescence | | | | | | interactions |
| | | kinase/fructose- | | | | | | | | |
| | | 2, 6- | | | | | | | | |
| | | bisphosphatase | | | | | | | | |
| C1402- | FMYEG | ATP-dependent | Mycobacterium | purified | dissociation | nM | 0.1 | | 2010 | Large scale analysis of |
| 04 | DTPL | helicase, | tuberculosis | MHC/direct/ | constant KD | | | | | peptide-HLA class I |
| | | putative | CDC1551 | fluorescence | | | | | | interactions |
| C1402- | MMHA | ornithine | Shewanella | purified | dissociation | nM | 0.1 | | 2010 | Large scale analysis of |
| 05 | STSPF | decarboxylase | baltica OS155 | MHC/direct/ | constant KD | | | | | peptide-HLA class I |
| | | | | fluorescence | | | | | | interactions |

TABLE 4
Conditional MHCI ligand sequences for
refolding screen.

| Peptide ID | Peptide seq |
|---|---|
| A0203-02 | FMYSDFHFI |
| A0203-02-01 | FJYSDFHFI |
| A0203-02-02 | FMYJDFHFI |
| A0203-02-03 | FMYSDJHFI |
| A0203-02-04 | FMYSDFHJI |
| A2601-01 | YVIRDLAAM |
| A2601-01-01 | YJIRDLAAM |
| A2601-01-02 | YVIJDLAAM |
| A2601-01-03 | YVIRDJAAM |
| A2601-01-04 | YVIRDLAJM |
| B1801-02 | DEFKPIVQY |
| B1801-02-01 | DJFKPIVQY |
| B1801-02-02 | DEFJPIVQY |
| B1801-02-03 | DEFKPJVQY |
| B1801-02-04 | DEFKPIVJY |
| B3503-04 | TPANYFHVL |
| B3503-04-01 | TJANYFHVL |
| B3503-04-02 | TPAJYFHVL |
| B3503-04-03 | TPANYJHVL |
| B3503-04-04 | TPANYFHJL |
| C0202-03 | FAAEAQKL |
| C0202-03-01 | FJAEAQKL |
| C0202-03-02 | FAAJAQKL |
| C0202-03-03 | FAAEAJKL |
| C0202-03-04 | FAAEAQKJ |
| C1402 | MMHASTSPF |
| C1402-05-01 | MJHASTSPF |
| C1402-05-02 | MMHJSTSPF |
| C1402-05-03 | MMHASJSPF |
| C1402-05-04 | MMHASTSJF |

### REFERENCES

1. Nielsen M, et al. (2003) Reliable prediction of T-cell epitopes using neural networks with novel sequence representations. Protein Sci. 12:1007-1017.
2. Andreatta M, and Nielsen, M. (2016) Gapped sequence alignment using artificial neural networks: application to the MHC class I system. Bioinformatics, Feb. 15; 32(4):511-517.
3. Chen, D. S., and Mellman, I. (2013) Oncology meets immunology: the cancer-immunity cycle. Immunity 39, 1-10.
4. van Rooij, N., van Buuren, M. M., Philips, D., Velds, A., Toebes, M., Heemskerk, B., van Dijk, L. J., Behjati, S., Hilkmann, H., El Atmioui, D., Nieuwland, M., Stratton, M. R., Kerkhoven, R. M., Kesmir, C., Haanen, J. B., Kvistborg, P., and Schumacher, T. N. (2013) Tumor exome analysis reveals neoantigen-specific T-cell reactivity in an ipilimumab-responsive melanoma. J Clin Oncol 31, e439-42.
5. Slaney, C. Y., Kershaw, M. H., and Darcy, P. K. (2014) Trafficking of T-cells into tumors. Cancer Res 74, 7168-74.
6. Melero, I., Rouzaut, A., Motz, G. T., and Coukos, G. (2014) T-cell and NK-cell infiltration into solid tumors: a key limiting factor for efficacious cancer immunotherapy. Cancer Discov 4, 522-6.
7. Zamora, A. E., Crawford, J. C., and Thomas, P. G. (2018) Hitting the Target: How T-cells Detect and Eliminate Tumors. J Immunol 200, 392-399.
8. Rosenberg, S. A. (2014) Decade in review-cancer immunotherapy: entering the mainstream of cancer treatment. Nat Rev Clin Oncol 11, 630-2.
9. Alsaab, H. O., Sau, S., Alzhrani, R., Tatiparti, K., Bhise, K., Kashaw, S. K., and Iyer, A. K. (2017) PD-1 and PD-L1 Checkpoint Signaling Inhibition for Cancer Immunotherapy: Mechanism, Combinations, and Clinical Outcome. Front Pharmacol 8, 561.
10. Choudhry, H., Helmi, N., Abdulaal, W. H., Zeyadi, M., Zamzami, M. A., Wu, W., Mahmoud, M. M., Warsi, M. K., Rasool, M., and Jamal, M. S. (2018) Prospects of IL-2 in Cancer Immunotherapy. Biomed Res Int 2018, 9056173.
11. Moran, A. E., Kovacsovics-Bankowski, M., and Weinberg, A. D. (2013) The TNFRs OX40, 4-1BB, and CD40 as targets for cancer immunotherapy. Curr Opin Immunol 25, 230-7.
12. Capietto, A. H., Jhunjhunwala, S., and Delamarre, L. (2017) Characterizing neoantigens for personalized cancer immunotherapy. Curr Opin Immunol 46, 58-65.
13. Hu, Z., Ott, P. A., and Wu, C. J. (2018) Towards personalized, tumour-specific, therapeutic vaccines for cancer. Nat Rev Immunol 18, 168-182.
14. Naidoo, J., Page, D. B., and Wolchok, J. D. (2014) Immune modulation for cancer therapy. Br J Cancer 111, 2214-9.
15. Palmer, D. C., Guittard, G. C., Franco, Z., Crompton, J. G., Eil, R. L., Patel, S. J., Ji, Y., Van Panhuys, N., Klebanoff, C. A., Sukumar, M., Clever, D., Chichura, A., Roychoudhuri, R., Varma, R., Wang, E., Gattinoni, L., Marincola, F. M., Balagopalan, L., Samelson, L. E., and Restifo, N. P. (2015) Cish actively silences TCR signaling in CD8+ T-cells to maintain tumor tolerance. J Exp Med 212, 2095-113.
16. Altman, J. D., Moss, P. A., Goulder, P. J., Barouch, D. H., McHeyzer-Williams, M. G., Bell, J. I., McMichael, A. J., and Davis, M. M. (1996) Phenotypic analysis of antigen-specific T lymphocytes. Science 274, 94-6.
17. Robbins, P. F., Lu, Y. C., El-Gamil, M., Li, Y. F., Gross, C., Gartner, J., Lin, J. C., Teer, J. K., Cliften, P., Tycksen, E., Samuels, Y., and Rosenberg, S. A. (2013) Mining exomic sequencing data to identify mutated antigens recognized by adoptively transferred tumor-reactive T-cells. Nat Med 19, 747-52.
18. Kvistborg, P., Shu, C. J., Heemskerk, B., Fankhauser, M., Thrue, C. A., Toebes, M., van Rooij, N., Linnemann, C., van Buuren, M. M., Urbanus, J. H., Beltman, J. B., Thor Straten, P., Li, Y. F., Robbins, P. F., Besser, M. J., Schachter, J., Kenter, G. G., Dudley, M. E., Rosenberg, S. A., Haanen, J. B., Hadrup, S. R., and Schumacher, T. N. (2012) TIL therapy broadens the tumor-reactive CD8(+) T-cell compartment in melanoma patients. Oncoimmunology 1, 409-418.
19. Chapuis, A. G., Lee, S. M., Thompson, J. A., Roberts, I. M., Margolin, K. A., Bhatia, S., Sloan, H. L., Lai, I., Wagener, F., Shibuya, K., Cao, J., Wolchok, J. D., Greenberg, P. D., and Yee, C. (2016) Combined IL-21-primed polyclonal CTL plus CTLA4 blockade controls refractory metastatic melanoma in a patient. J Exp Med 213, 1133-9.
20. Garboczi, D. N., Hung, D. T., and Wiley, D. C. (1992) HLA-A2-peptide complexes: refolding and crystallization of molecules expressed in Escherichia coli and complexed with single antigenic peptides. Proc Natl Acad Sci USA 89, 3429-33.
21. Robinson, J., Barker, D. J., Georgiou, X., Cooper, M. A., Flicek, P., and Marsh, S. G. E. (2020) IPD-IMGT/HLA Database. Nucleic Acids Res 48, D948-D955.
22. Rodenko, B., Toebes, M., Hadrup, S. R., van Esch, W. J., Molenaar, A. M., Schumacher, T. N., and Ovaa, H. (2006) Generation of peptide-MHC class I complexes through UV-mediated ligand exchange. Nat Protoc 1, 1120-32.
23. Brackenridge, S., Evans, E. J., Toebes, M., Goonetilleke, N., Liu, M. K., di Gleria, K., Schumacher, T. N., Davis, S. J., McMichael, A. J., and Gillespie, G. M. (2011) An early HIV mutation within an HLA-B*57-restricted T-cell epitope abrogates binding to the killer inhibitory receptor 3DL1. J Virol 85, 5415-22.
24. Frosig, T. M., Yap, J., Seremet, T., Lyngaa, R., Svane, I. M., Thor Straten, P., Heemskerk, M. H., Grotenbreg, G. M., and Hadrup, S. R. (2015) Design and validation of conditional ligands for HLA-B*08:01, HLA-B*15:01, HLA-B*35:01, and HLA-B*44:05. Cytometry A 87, 967-75.
25. Chang, C. X., Tan, A. T., Or, M. Y., Toh, K. Y., Lim, P. Y., Chia, A. S., Froesig, T. M., Nadua, K. D., Oh, H. L., Leong, H. N., Hadrup, S. R., Gehring, A. J., Tan, Y. J., Bertoletti, A., and Grotenbreg, G. M. (2013) Conditional ligands for Asian HLA variants facilitate the definition of CD8+ T-cell responses in acute and chronic viral diseases. Eur J Immunol 43, 1109-20.
26. Toebes, M., Coccoris, M., Bins, A., Rodenko, B., Gomez, R., Nieuwkoop, N. J., van de Kasteele, W., Rimmelzwaan, G. F., Haanen, J. B., Ovaa, H., and Schumacher, T. N. (2006) Design and use of conditional WIC class I ligands. Nat Med 12, 246-51.
27. Bakker, A. H., Hoppes, R., Linnemann, C., Toebes, M., Rodenko, B., Berkers, C. R., Hadrup, S. R., van Esch, W. J., Heemskerk, M. H., Ovaa, H., and Schumacher, T. N. (2008) Conditional MHC class I ligands and peptide exchange technology for the human WIC gene products HLA-A1, -A3, -A11, and -B7. Proc Natl Acad Sci USA 105, 3825-30.
28. Bui, H. H., Sidney, J., Dinh, K., Southwood, S., Newman, M. J., and Sette, A. (2006) Predicting population coverage of T-cell epitope-based diagnostics and vaccines. BMC Bioinformatics 7, 153.
29. Sylvester-Hvid, C., Kristensen, N., Blicher, T., Ferre, H., Lauemoller, S. L., Wolf, X. A., Lamberth, K., Nissen, M. H., Pedersen, L. O., and Buus, S. (2002) Establishment of a quantitative ELISA capable of determining peptide—WIC class I interaction. Tissue Antigens 59, 251-8.
30. Pedersen, L. E., Harndahl, M., Rasmussen, M., Lamberth, K., Golde, W. T., Lund, O., Nielsen, M., and Buus, S. (2011) Porcine major histocompatibility complex (WIC) class I molecules and analysis of their peptide-binding specificities. Immunogenetics 63, 821-34.
31. Darwish, M., Shatz, W., Leonard, B., Loyet, K., Barrett, K., Wong, J. L., Li, H., Abraham, R., Lin, M., Franke, Y., Tam, C., Mortara, K., Zilberleyb, I., and Blanchette, C. (2020) Nanolipoprotein Particles as a Delivery Platform for Fab Based Therapeutics. Bioconjug Chem 31, 1995-2007.
32. Harndahl, M., Rasmussen, M., Roder, G., Dalgaard Pedersen, I., Sorensen, M., Nielsen, M., and Buus, S. (2012) Peptide-WIC class I stability is a better predictor than peptide affinity of CTL immunogenicity. Eur J Immunol 42, 1405-16.
33. Bouvier, M., and Wiley, D. C. (1994) Importance of peptide amino and carboxyl termini to the stability of WIC class I molecules. Science 265, 398-402.
34. Ferre, H., Ruffet, E., Blicher, T., Sylvester-Hvid, C., Nielsen, L. L., Hobley, T. J., Thomas,
0. R., and Buus, S. (2003) Purification of correctly oxidized MHC class I heavy-chain molecules under denaturing conditions: a novel strategy exploiting disulfide assisted protein folding. Protein Sci 12, 551-9.
35. Saini, S. K., Ostermeir, K., Ramnarayan, V. R., Schuster, H., Zacharias, M., and Springer, S. (2013) Dipeptides promote folding and peptide binding of MHC class I molecules. Proc Natl Acad Sci USA 110, 15383-8.
36. Cole, D. K., Edwards, E. S., Wynn, K. K., Clement, M., Miles, J. J., Ladell, K., Ekeruche, J., Gostick, E., Adams, K. J., Skowera, A., Peakman, M., Wooldridge, L., Price, D. A., and Sewell, A. K. (2010) Modification of MHC anchor residues generates heteroclitic peptides that alter TCR binding and T-cell recognition. J Immunol 185, 2600-10.
37. Sims, S., Willberg, C., and Klenerman, P. (2010) WIC-peptide tetramers for the analysis of antigen-specific T-cells. Expert Rev Vaccines 9, 765-74.
38. Luimstra, J. J., Garstka, M. A., Roex, M. C. J., Redeker, A., Janssen, G. M. C., van Veelen, P. A., Arens, R., Falkenburg, J. H. F., Neefjes, J., and Ovaa, H. (2018) A flexible WIC class I multimer loading system for large-scale detection of antigen-specific T-cells. J Exp Med 215, 1493-1504.
39. Wingfield, P. T. (2017) N-Terminal Methionine Processing. Curr Protoc Protein Sci 88, 6 14 1-6 14 3.
40. Hadrup, S. R., Toebes, M., Rodenko, B., Bakker, A. H., Egan, D. A., Ovaa, H., and Schumacher, T. N. (2009) High-throughput T-cell epitope discovery through MHC peptide exchange. Methods Mol Biol 524, 383-405.
41. Toebes, M., Rodenko, B., Ovaa, H., and Schumacher, T. N. (2009) Generation of peptide MHC class I monomers and multimers through ligand exchange. Curr Protoc Immunol Chapter 18, Unit 18 16.

1. A major histocompatibility complex class I (MHCI)/ligand complex comprising (i) a MHCI molecule comprising an alpha chain, a beta chain, and (ii) a ligand, wherein the ligand is a peptide comprising a non-natural UV-cleavable amino acid.

2-13. (canceled)

14. A peptide exchange assay for determining binding of a major histocompatibility complex class I (MHCI) allele to a test peptide, comprising:
(a) providing a first composition comprising a test peptide and a MHCI/ligand complex comprising (i) a MHCI molecule comprising an alpha chain, a beta chain, and (ii) a ligand, wherein the ligand is a peptide comprising a non-natural ultraviolet (UV)-cleavable amino acid;
(b) exposing the first composition to UV light to cleave the ligand at the UV-cleavable amino acid; and
(c) incubating the first composition for a period of time to form a second composition comprising free test peptide, the alpha chain, the beta chain, and/or a MHCI/-second peptide complex; and
(d) determining whether the MHCI allele is bound to the second peptide.

15. The peptide exchange assay of claim 14, wherein MHCI allele binding to the peptide is determined by measuring a level of MHCI/peptide complex in the second composition.

16. The peptide exchange assay of claim 15, wherein the level of MHCI/second peptide complex is measured by 2-dimensional liquid chromatography-mass spectrometry (2D LC/MS) of the second composition.

17. The peptide exchange assay of claim 16, wherein 2D LC/MS comprises removing the free second peptide from the second composition.

18. The peptide exchange assay of claim 14, further comprising performing high-performance liquid chromatography (HPLC) and mass spectrometry (MS) to distinguish the MHCI and the second peptide.

19. The peptide exchange assay of claim 14, wherein the free second peptide is removed from the second composition by separation via size exclusion chromatography.

20. The peptide exchange assay of claim 18, wherein presence of the second peptide as determined by HPLC and MS indicates that the MHCI is capable of binding to the second peptide.

21. The peptide exchange assay of claim 14, wherein a plurality of the MHCI/ligand complex is combined with at least two different test peptides.

22. The peptide exchange assay of claim 21, wherein the test peptides are identified by mass spectrometry based on the predicted mass of each peptide.

23. The peptide exchange assay of claim 14, wherein the test peptide is present in the first composition at a ratio of at least 10:1 (test peptide:MHCI).

24. The peptide exchange assay of claim 14, wherein the MHCI/ligand complex is a MHCI/ligand complex of any one of claims 1 to 13.

25. The peptide exchange assay of claim 14, wherein the MHCI/peptide complex further comprises a first label thereby forming a labeled MHCI/peptide complex.

26. The peptide exchange assay of claim 25, wherein the level of peptide exchange is determined by:
(a) contacting the labeled MHCI/ligand peptide complex with:
(i) an antibody complex comprising an anti-MHCI allele antibody covalently attached to a fluorescence resonance energy transfer (FRET) acceptor; and
(ii) a FRET emitter complex comprising a FRET emitter conjugated to a second label, thereby forming a reaction composition;
(b) detecting FRET emission of the second label in the reaction composition, thereby detecting binding of a MHCI allele to a peptide.

27. The peptide exchange assay of claim 26, wherein the reaction composition is incubated for at least about 10 hours or at least about 15 hours.

28. (canceled)

29. The peptide exchange assay of claim 26, wherein the first label and the second label are independently streptavidin or biotin.

30. The peptide exchange assay of claim 26, wherein the anti-MHCI allele antibody comprises an anti-HLA antibody, monobody, or partial antibody.

31-33. (canceled)

34. The peptide exchange assay of claim 26, wherein emission from the FRET acceptor indicates binding of the test peptide to the MHCI.

35. The peptide exchange assay of claim 26, wherein binding of the MHCI to the test peptide is confirmed by a second peptide exchange assay.

36. The peptide exchange assay of claim 35, wherein the second peptide exchange assay is a peptide exchange assay of claim 15.

37. The peptide exchange assay of claim 26, wherein the amount of test peptide binding to the MHCI molecule is determined by TR-FRET.

38. A method of detecting binding of a major histocompatibility complex class I (MHCI) allele to a test peptide, the method comprising:
(a) providing a first composition comprising a test peptide and a MHCI/ligand complex comprising (i) a MHCI molecule comprising an alpha chain, a beta chain, and (ii) a ligand, wherein the ligand is a peptide comprising a non-natural ultraviolet (UV)-cleavable amino acid;
(b) exposing the first composition to UV light to cleave the ligand at the UV-cleavable amino acid; and
(c) detecting a MHCI/test peptide complex in the second composition, thereby detecting binding of the MHCI molecule to the test peptide.

39-44. (canceled)

45. A method of identifying a MHCI binding ligand; the method comprising:
(a) contacting a plurality of MHCI alpha chain monomers with a plurality of beta chain monomers and a ligand under conditions that allow for the formation of a MHCI/ligand complex, wherein the ligand is a peptide comprising a non-natural UV-cleavable amino acid; and
(b) detecting the MHCI/ligand complex, thereby identifying a MHCI binding ligand.

46-51. (canceled)

52. A method for determining optimal major histocompatibility complex class I (MHCI) allele-ligand combinations, the method comprising:
(a) providing a plurality of MHCI alpha chain monomers purified under denaturing conditions;
(b) forming a reaction mixture by combining the plurality of MHCI alpha chain monomers, a plurality of beta chain monomers, and a ligand comprising a peptide comprising a non-natural UV-cleavable amino acid;
(c) incubating the mixture under conditions to allow formation of a MHCI/ligand complex; and
(d) determining whether the MHCI/ligand complex was formed.

53. The method of claim 52, wherein the plurality of MHCI alpha chain monomers, plurality of beta chain monomers, and ligand are incubated for at least 48 hours or for about 5 days.

54. (canceled)

55. The method of claim 52, wherein a plurality of ligands are screened, wherein each ligand comprises an amino acid sequence, wherein the amino acid sequence of each ligand differs from the amino acid sequence of each other ligand only by the position of the UV-cleavable amino acid in the sequence.

56. The method of claim 52, wherein step d) comprises performing an enzyme-linked immunosorbent assay (ELISA).

57. The method of claim 56, wherein the ELISA comprises (i) introducing the reaction mixture into a container, the container comprising a surface and an anti-MHCI alpha chain antibody conjugated to the surface; (ii) introducing a labeled anti-beta chain antibody comprising a detectable label into the container, such that the labeled anti-beta chain antibody binds the beta chain monomers, if present; (iii) washing to remove unbound labeled anti-beta chain antibody; and (iv) detecting the presence of the detectable label in the container.

58. The method of claim 57, wherein the detectable label comprises biotin or a peptide tag.

59. The method of claim 57, wherein the detectable label comprises biotin.

60. The method of claim 57, wherein step (iv) comprises introducing a streptavidin-horseradish peroxidase (HRP) conjugate into the container and determining a level of chemiluminescence upon addition of a HRP substrate.

61. The method of claim 57, wherein the container is a well of a multi-well plate.

62. The method of claim 52, wherein steps a) through d) are performed separately for at least two ligands, wherein step d) comprises determining a level of MHCI/ligand complex formation, wherein the ligand having the greatest level of MHCI/ligand complex formation is the optimal MHCI/ligand combination for the MHCI allele.

63. A peptide comprising a non-natural UV-cleavable amino acid, wherein the peptide has an amino acid sequence of any one of SEQ ID NO.: 1 to SEQ ID NO.: 34.

64. A method of monitoring peptide-exchanged major histocompatibility class I (MHCI) complexes in a sample, comprising:
(a) obtaining peptide-exchanged MHCI complexes comprising a peptide, wherein the peptide is
(i) a peptide of interest, or
(ii) an exchangeable peptide and exposing the complexes to one or more peptides of interest under conditions which allow for peptide exchange between the exchangeable peptide and the peptide of interest;
(b) performing size exclusion chromatography (SEC), capillary electrophoresis (CE), or capillary zone electrophoresis (CZE) on the peptide exchanged MHCI complexes; and
(c) following the chromatography or capillary electrophoresis of (b), performing native mass spectrometry (MS) on the MHCI complexes to identify MHCI complexes that comprise peptides of interest.

65. (canceled)

66. A method of monitoring T-cell recognition of MHCI-complexed peptides, comprising:
(a) obtaining peptide-exchanged MHCI complexes comprising a peptide, wherein the peptide is
(i) a peptide of interest, or
(ii) an exchangeable peptide and exposing the complexes to one or more peptides of interest under conditions which allow for peptide exchange between the exchangeable peptide and the peptide of interest;
(b) contacting the peptide-exchanged MHCI complexes with a sample comprising T-cells;
(c) separating T-cell bound MHCI complexes from unbound MHCI complexes;
(d) performing size exclusion chromatography (SEC), capillary electrophoresis (CE), or capillary zone electrophoresis (CZE) on the peptide exchanged MHCI complexes; and
(e) following the chromatography or capillary electrophoresis of (d), performing native mass spectrometry (MS) on the MHCI complexes to identify MHCI complexes comprising peptides recognized by T-cells from the sample.

67-86. (canceled)

87. A kit comprising a peptide comprising a non-natural UV-cleavable amino acid, MHCI alpha chain monomers, and MHCI beta chain monomers. complex.

88-94. (canceled)

95. A system comprising:
(a) a peptide comprising a non-natural UV-cleavable amino acid;
(b) a plurality of MHCI alpha chain monomers;
(c) a plurality of MHCI beta chain monomers;
(d) and a first reagent capable of allowing formation of a MHCI/ligand complex.

96-99. (canceled)
